(19) United States
(12) Patent Application Publication
(10) Pub. No.: US 2022/0073915 A1
(43) Pub. Date: Mar. 10, 2022

(54) ARTIFICIAL NUCLEIC ACIDS FOR RNA EDITING

(71) Applicant: Eberhard-Karls-Universität-Tübingen, Tübingen (DE)

(72) Inventors: Jacqueline Wettengel, Sigmaringen (DE); Thorsten Stafforst, Tübingen (DE); Philipp Reautschnig, Tübingen (DE); Tobias Merkle, Tübingen (DE)

(21) Appl. No.: 17/256,092

(22) PCT Filed: Jun. 29, 2018
(86) PCT No.: PCT/EP2018/067718

§ 371 Date: Dec. 24, 2020

(51) Int. Cl. C12N 15/113; C12N 9/78

(52) U.S. Cl. CPC C12N 15/113; C12N 9/78; C12N 2310/346; C12N 2310/321; C12N 2310/3231; C12Y 305/04001; C12N 2310/315; C12N 2310/531; C12N 2310/533; C12Y 305/04004; C12N 2310/322

(57) ABSTRACT

The present invention concerns artificial nucleic acids for site-directed editing of a target RNA. In particular, the present invention provides artificial nucleic acids capable of site-directed editing of endogenous transcripts by harnessing an endogenous deaminase. Further, the present invention provides artificial nucleic acids for sited-directed editing of a target RNA, which are chemically modified, in particular according to a modification pattern as described herein. The invention also comprises a vector encoding said artificial nucleic acid and a composition comprising said artificial nucleic acid. Moreover, the invention provides the use of the artificial nucleic acid, the composition or the vector for site-directed editing of a target RNA or for in vitro diagnosis. In addition, the artificial nucleic acid, the composition or the vector as described herein are provided for use as a medicament or for use in diagnosis of a disease or disorder.

The present invention concerns artificial nucleic acids for site-directed editing of a target RNA. In particular, the present invention provides artificial nucleic acids capable of site-directed editing of endogenous transcripts by harnessing an endogenous deaminase. Further, the present invention provides artificial nucleic acids for sited-directed editing of a target RNA, which are chemically modified, in particular according to a modification pattern as described herein. The invention also comprises a vector encoding said artificial nucleic acid and a composition comprising said artificial nucleic acid. Moreover, the invention provides the use of the artificial nucleic acid, the composition or the vector for site-directed editing of a target RNA or for in vitro diagnosis. In addition, the artificial nucleic acid, the composition or the vector as described herein are provided for use as a medicament or for use in diagnosis of a disease or disorder.

### BACKGROUND OF THE INVENTION

In conventional gene therapy, the genetic information is typically manipulated at the DNA level, thus permanently altering the genome. Depending on the application, the persistent modification of the genome may be either advantageous or imply serious risks. In this respect, the targeting of RNA instead of DNA represents an attractive alternative approach. When treating a subject on the RNA level, the change in gene expression is usually reversible, tunable and very frequently also more efficient. On the one hand, the limited duration of the effect will also limit the risks related to harmful side-effects. In addition, the possibility to finely tune the effect allows for continuously adjusting the therapy and control the adverse effects in a time and dose-dependent manner. Furthermore, many manipulations of gene expression are not feasible or ineffective at the genome level, e.g. when the gene loss is either lethal or readily compensated by redundant processes. For example, it appears particularly attractive to target signaling networks at the RNA level. Many signaling cues are either essential, or they are strongly redundant so that a knockout sometimes does not result in a clear phenotype while a knockdown does.

Accordingly, there is an increasing interest in the engineering of RNA targeting strategies. One such strategy is RNA editing. (A)denosine-to-(I)nosine RNA editing is a natural enzymatic mechanism to diversify the transcriptome. Since inosine is biochemically interpreted as guanosine, A-to-I editing formally introduces A-to-G mutations, which can result in the recoding of amino acid codons, START and STOP codons, alteration of splicing, and alteration of miRNA activity, amongst others. Targeting such enzyme activities to specific sites at selected transcripts, a strategy called site-directed RNA editing, holds great promise for the treatment of disease and the general study of protein and RNA function. RNA editing strategies based on engineered deaminases were developed (see, for example, Vogel, P., Schneider, M. F., Wettengel, J., Stafforst, T. Improving Site-Directed RNA Editing In Vitro and in Cell Culture by Chemical Modification of the GuideRNA. Angew. Chem. Int. Ed. 53, 6267-6271 (2014). However, in a therapeutic setting, the harnessing of the widely expressed, endogenous deaminases acting on RNA would be the most attractive. It would allow for introducing a specific mutation into the transcriptome by administration of an oligonucleotide drug only, without the need for the ectopic expression of any (engineered) protein. For instance, Wettengel et al. (Wettengel, J., Reautschnig, J., Geisler, S., Kahle, P. J., Stafforst, T. Harnessing human ADAR2 for RNA repair—Recoding a PINK1 mutation rescues mitophagy. Nucl. Acids Res. 45, 2797-2808 (2017)) reported a system that requires no artificial protein, but employs cellular ADAR2. Moreover, oligonucleotide constructs for site-directed RNA editing are described in international patent applications WO 2016/097212 and WO 2017/010556. Also German patent DE 10 2015 012 522 B3 describes a guideRNA molecule for site-directed RNA editing.

However, the strategies known in the art suffer from similar problems: on the one hand, it proved difficult to recruit deaminase, in particular endogenous deaminase, efficiently enough in order to provide for sufficient RNA editing. On the other hand, efficient editing typically comes along with low specificity, e.g. numerous off-target editings all over the transcriptome. This is particularly true when a known hyperactive mutant (Kuttan A, Bass BL: Mechanistic insights into editing-site specificity of ADARs. Proceedings of the National Academy of Sciences 2012, 109:E3295-E3304), called E/Q herein, is applied to improve efficiency and codon scope.

There is therefore an urgent need of RNA editing strategies that allow for high editing yields and high specificity. In particular, compounds are required that are suitable for recruiting endogenous deaminases and which do not result in off-target editing.

It is thus an objective of the present invention to provide a compound that is capable of recruiting a deaminase, preferably an endogenous deaminase, e.g. an adenosine deaminase, to an RNA target to be edited. A particular objective of the present invention is the provision of a compound suitable for editing an RNA target with high efficiency and high specificity, in particular with a reduced rate of off-target editing. Improved RNA editing approaches shall thus be provided, which allow for high yields of RNA editing at a specifically targeted site in a target RNA, preferably without or with reduced unspecific editing at other genomic sites. Another particular objective of the present invention is the provision of an RNA editing system, preferably characterized by the afore-mentioned advantages, which harnesses endogenous deaminases.

The solution of said object is achieved by the embodiments described herein and defined by the claims.

### DETAILED DESCRIPTION OF THE INVENTION

Artificial Nucleic Acids for Site-Directed RNA Editing

In a first aspect, the present invention concerns novel artificial nucleic acids for site-directed editing of a target RNA. In particular, an artificial nucleic acid for site-directed editing of a target RNA is provided herein, the artificial nucleic acid comprising
a) a targeting sequence, which comprises a nucleic acid sequence complementary or partially complementary to a target sequence in the target RNA,
   and
b) a recruiting moiety for recruiting a deaminase,
   wherein the targeting sequence comprises at least one nucleotide, wherein the nucleobase is chemically modified,
   and/or
   wherein the targeting sequence comprises at least one backbone modification.

The inventors surprisingly found that the artificial nucleic acids as described herein, in particular an artificial nucleic acid comprising a targeting sequence that is chemically modified as defined herein, are capable of recruiting deaminases, particularly endogenous deaminases, to an RNA target and to specifically edit a nucleotide, preferably an adenosine or a cytidine nucleotide, at a target site in said RNA. Advantageously, the target RNA is edited by the artificial nucleic acid described herein with high efficiency, thus providing for high yields of edited target RNA. Surprisingly, an increased RNA editing yield is achieved by using the artificial nucleic acid, while undesired off-target editing can nevertheless be avoided. The artificial nucleic acid described herein thus allows for site-directed RNA editing with both, high efficiency as well as high specificity. The inventors have found that the artificial nucleic acid is suitable for editing a wide variety of transcripts, e.g. endogenous mRNAs of housekeeping genes as well as endogenous transcripts of disease-related genes (such as STAT1 or SERPINA1). Advantageously, the system according to the present invention proved to be applicable to a large variety of cells, ranging from immortalized cell lines and tumour cell lines to several primary human cells. The inventors further observed that the artificial nucleic acid according to the invention is also particularly resistant to degradation, for example, in serum. Without wishing to be bound to any hypothesis, it is believed that the improved stability of the artificial nucleic acid described herein contributes to the advantageous effects described above.

As used herein, the phrase ‘artificial nucleic acid (molecule)’ typically refers to a nucleic acid that does not occur naturally. In other words, an artificial nucleic acid molecule may be a non-natural nucleic acid. Such an artificial nucleic acid molecule may be non-natural due to its individual sequence (which does not occur naturally) and/or due to other modifications, e.g. structural modifications of nucleotides, which do not occur naturally in that context. An artificial nucleic acid as used herein preferably differs from a naturally occurring nucleic acid by at least one nucleotide or by at least one modification of a nucleotide. An artificial nucleic acid molecule may be a DNA molecule, an RNA molecule or a hybrid-molecule comprising DNA and RNA portions. In preferred embodiments, the artificial nucleic acid is an RNA molecule, which preferably comprises one or more 2′-deoxynucleotides. In particular, an artificial nucleic acid as used herein may comprise (unmodified or modified) ribonucleotides and/or (unmodified or modified) deoxynucleotides. Typically, an artificial nucleic acid may be designed and/or generated by genetic engineering methods, so as to correspond to a desired artificial sequence of nucleotides (heterologous sequence) or to a nucleic acid sequence having a desired artificial modification pattern as described herein. Further, the phrase ‘artificial nucleic acid (molecule)’ is not restricted to ‘one single molecule’ but may also refer to an ensemble of identical molecules. Accordingly, the phrase may refer to a plurality of identical molecules contained, for example, in a sample.

In the context of the present invention, the phrase ‘RNA editing’ refers the reaction, by which a nucleotide, preferably an adenosine or a cytidine nucleotide, in a target RNA is transformed by a deamination reaction into another nucleotide. That change typically results in a different gene product, since the changed nucleotide preferably results in a codon change, leading e.g. to incorporation of another amino acid in the polypeptide translated from the RNA or to the generation or deletion of a stop codon. In particular, an adenosine nucleotide in a target RNA is converted to inosine by deamination, e.g. by an adenosine deaminase as described herein. In an alternative embodiment, a cytidine nucleotide in a target RNA is converted to an uridine nucleotide. As used herein, the term ‘target RNA’ typically refers to an RNA, which is subject to the editing reaction, which is supported by the artificial nucleic acid described herein.

The RNA editing achieved by the artificial nucleic acid described herein is further ‘site-directed’, which means that a specific nucleotide at a target site in a target RNA is edited, preferably without or essentially without editing other nucleotides. Typically, the nucleotide at the target site is targeted by the targeting sequence of the artificial nucleic acid described herein, wherein the targeting sequence is capable of specific base-pairing with the target sequence, preferably under physiological conditions. In the context of the present invention, the phrase ‘target sequence’ is thus typically used with regard to the nucleic acid sequence, which is (at least partially) complementary to the targeting sequence of the artificial nucleic acid. The target sequence comprises the target site, wherein the target site is typically a nucleotide, preferably an adenosine or a cytidine nucleotide, to be edited. In some embodiments, a target site my comprise two or more nucleotides to be edited, wherein these nucleotides are preferably from each other by at least one, preferably two, other nucleotides. As used herein, the terms ‘complementary’ or ‘partially complementary’ preferably refer to nucleic acid sequences, which due to their complementary nucleotides are capable of specific intermolecular base-pairing, preferably Watson-Crick base pairing, preferably under physiological conditions. The term ‘complementary’ as used herein may also refer to reverse complementary sequences. The artificial nucleic acid described herein may also be referred to herein as ‘antisense oligonucleotide’ or ‘ASO’, as the artificial nucleic acid typically comprises a nucleic acid sequence in the targeting sequence, which represents the antisense of a nucleic acid sequence in the target RNA. The targeting sequence thus preferably directs the recruiting moiety and the deaminase towards the target site in a target RNA in a sequence-specific manner. In the context of the present invention, the term ‘guideRNA’ may also be used in order to refer to the artificial nucleic acid, which preferably guides the deaminase function to the target site.

In the context of the present invention, the term ‘recruiting moiety’ refers to a moiety of the artificial nucleic acid described herein, which recruits the deaminase and which is typically covalently linked to the targeting sequence. The ‘recruiting moiety’ thus recruits a deaminase to the target site in a target RNA, wherein the target RNA (and the target site) are preferably recognized and bound in a sequence-specific manner by the targeting sequence. In certain embodiments, the recruiting moiety comprises or consists of at least one coupling agent capable of recruiting a deaminase, wherein the deaminase comprises a moiety that binds to said coupling agent. The coupling agent, which recruits a deaminase is typically covalently linked to the targeting sequence. Preferably, the coupling agent is linked to the 5′-terminus or to the 3′-terminus of the targeting sequence. The coupling agent may alternatively also be linked to an internal nucleotide (i.e. not a 5′- or 3′-terminal nucleotide) of the targeting sequence, for example via linkage to a nucleotide variant or a modified nucleotide, preferably as described herein, such as amino-thymidine. In a further embodiment, the recruiting moiety comprises a nucleic acid sequence, which is capable of specifically binding to a deaminase, preferably to a double-stranded (ds) RNA binding domain of a deaminase. Said nucleic acid sequence of the recruiting moiety is typically linked covalently either to the 5′ terminus or to the 3′ terminus of the targeting sequence, preferably to the 5′ terminus of the targeting sequence. In certain embodiments, the artificial nucleic acid as described herein comprises a targeting sequence as described herein and at least two recruiting moieties as described herein.

In some embodiments, the artificial nucleic acid comprises a moiety, which enhances cellular uptake of the artificial nucleic acid. Preferably, the moiety enhancing cellular uptake is a triantennary N-acetyl galactosamine (GalNAc3), which is preferably conjugated with the 3′ terminus or with the 5′ terminus of the artificial nucleic acid.

The artificial nucleic acid according to the present invention is not limited in its length and may be, for example, an oligonucleotide. As used herein, the term ‘oligonucleotide’ may refer to short nucleic acid molecules (e.g. a 6-mer or a 10-mer) as well as to longer oligonucleotides (e.g. nucleic acid molecules comprising 100 or even 200 nucleotides), wherein the oligonucleotide may comprise (unmodified or modified) ribonucleotides and/or (unmodified or modified) deoxynucleotides. According to a preferred embodiment, the artificial nucleic acid comprises at least about 15, preferably at least about 20, more preferably at least about 25, even more preferably at least about 30, even more preferably at least about 35, most preferably at least about 40, nucleotides. Alternatively, the length of the artificial nucleic acid is in the range from about 10 to about 200 nucleotides, preferably from about 15 to about 100 nucleotides, more preferably from about 15 to about 70 nucleotides, most preferably from about 20 to about 70 nucleotides.

The artificial nucleic acid as described herein is preferably a single-stranded (ss) nucleic acid molecule. In a preferred embodiment, the artificial nucleic acid is a single-stranded nucleic acid, which at physiological conditions comprises double-stranded (ds) regions. Preferably, the artificial nucleic acid is a single-stranded nucleic acid comprising double-stranded regions within the recruiting moiety.

The targeting sequence of the artificial nucleic acid typically comprises a nucleic acid sequence complementary or at least partially complementary to a nucleic acid sequence in the target RNA, preferably to a nucleic acid sequence immediately 5′ and to a nucleic acid sequence immediately 3′ of the nucleotide at the target site. Preferably, the targeting sequence comprises a nucleic acid sequence complementary or at least 60%, 70%, 80%, 90%, 95% or 99% complementary to a nucleic acid sequence in the target RNA, wherein the complementary nucleic acid sequence in the target RNA comprises the target site and preferably comprises at least 10, at least 12, at least 15, at least 18, at least 20, at least 22, at least 25 or at least 30 nucleotides. Preferably, the targeting sequence of the artificial nucleic acid is present as an essentially single-stranded nucleic acid, in particular under physiological conditions.

The artificial nucleic acid as described herein may be synthesized by a method known in the art. Preferably, the artificial nucleic acid is synthesized chemically or by in vitro transcription from a suitable vector, preferably as described herein. If not stated otherwise, the nucleic acid sequences provided herein are printed from 5′ to 3′. In other terms, the first nucleotide residue in a nucleic acid sequence printed herein is—if not stated otherwise—the 5′-terminus of said nucleic acid sequence. Amino acid sequences—if not stated otherwise—are printed from the N-terminus to the C-terminus.

Chemical Modifications

The artificial nucleic acids according to the present invention are typically chemically modified. As used herein, the term ‘chemical modification’ preferably refers to a chemical modification selected from backbone modifications, sugar modifications or base modifications, including abasic sites. A ‘chemically modified nucleic acid’ in the context of the present invention may refer to a nucleic acid comprising at least one chemically modified nucleotide.

The artificial nucleic acid preferably comprises a targeting sequence comprising at least one chemically modified nucleotide. More preferably, the targeting sequence comprises a plurality of chemically modified nucleotides, preferably resulting a modification pattern of the targeting sequence as described herein. In an alternative embodiment, the artificial nucleic acid comprises a recruiting moiety comprising a nucleic acid sequence capable of specifically binding to a deaminase, wherein the recruiting moiety comprises at least one chemically modified nucleotide. In a preferred embodiment, the nucleic acid sequence in the recruiting moiety comprises a plurality of chemically modified nucleotides, preferably resulting a modification pattern of the nucleic acid sequence of the recruiting moiety as described herein. According to a particularly preferred embodiment, the artificial nucleic acid comprises a chemically modified targeting sequence as described herein and a recruiting moiety comprising a chemically modified nucleic acid sequence as described herein.

Generally, the artificial nucleic acid molecule of the present invention may comprise native (=naturally occurring) nucleotides as well as chemically modified nucleotides. As used herein, the term ‘nucleotide’ generally comprises (unmodified and modified) ribonucleotides as well as (unmodified and modified) deoxynucleotides. The term ‘nucleotide’ thus preferably refers to adenosine, deoxyadenosine, guanosine, deoxyguanosine, 5-methoxyuridine, thymidine, uridine, deoxyuridine, cytidine, deoxycytidine or to a variant thereof. Moreover, where reference is made herein to a ‘nucleotide’, the respective nucleoside is preferably comprised as well.

In this respect, a ‘variant’ of a nucleotide is typically a naturally occurring or an artificial variant of a nucleotide. Accordingly, variants are preferably chemically derivatized nucleotides with non-natively occurring functional groups, which are preferably added to or deleted from the naturally occurring nucleotide or which substitute the naturally occurring functional groups of a nucleotide. Accordingly, in such a nucleotide variant each component of the naturally occurring nucleotide, preferably a ribonucleotide or a deoxynucleotide, may be modified, namely the base component, the sugar (ribose) component and/or the phosphate component forming the backbone of the artificial nucleic acid, preferably by a modification as described herein. The term ‘variant (of a nucleotide, ribonucleotide, deoxynucleotide, etc.)’ thus also comprises a chemically modified nucleotide, preferably as described herein.

A chemically modified nucleotide as used herein is preferably a variant of guanosine, uridine, adenosine, thymidine and cytosine including, without implying any limitation, any natively occurring or non-natively occurring guanosine, uridine, adenosine, thymidine or cytidine that has been altered chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2′-O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. The preparation of such variants is known to the person skilled in the art, for example from U.S. Pat. Nos. 4,373,071, 4,401,796, 4,415,732, 4,458,066, 4,500,707, 4,668,777, 4,973,679, 5,047,524, 5,132,418, 5,153,319, 5,262,530 or 5,700,642.

In some embodiments, the artificial nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, puromycin-5′-triphosphate, or xanthosine-5′-triphosphate.

In some embodiments, the artificial nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, the artificial nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, the artificial nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In certain embodiments, the artificial nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

According to a preferred embodiment, the artificial nucleic acid comprises at least one chemically modified nucleotide, which is chemically modified at the 2′ position. Preferably, the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. In the context of the artificial nucleic acid, in particular if the artificial nucleic acid is an RNA or a molecule comprising ribonucleotides, a 2′-deoxynucleotide (comprising hydrogen as a substituent at the 2′ carbon atom), such as deoxycytidine or a variant thereof, may also be referred to as ‘chemically modified nucleotide’.

Another chemical modification that involves the 2′ position of a nucleotide as described herein is a locked nucleic acid (LNA) nucleotide, an ethylene bridged nucleic acid (ENA) nucleotide and an (S)-constrained ethyl cEt nucleotide. These backbone modifications lock the sugar of the modified nucleotide into the preferred northern conformation. It is believed that the presence of that type of modification in the targeting sequence of the artificial nucleic acid allows for stronger and faster binding of the targeting sequence to the target RNA.

According to some embodiments, the artificial nucleic acid comprises at least one chemically modified nucleotide, wherein the phosphate backbone, which is incorporated into the artificial nucleic acid molecule, is modified. The phosphate groups of the backbone can be modified, for example, by replacing one or more of the oxygen atoms with a different substituent. Further, the modified nucleotide can include the full replacement of an unmodified phosphate moiety with a modified phosphate as described herein. Examples of modified phosphate groups include, but are not limited to, the group consisting of a phosphorothioate, a phosphoroselenate, a borano phosphate, a borano phosphate ester, a hydrogen phosphonate, a phosphoroamidate, an alkyl phosphonate, an aryl phosphonate and a phosphotriester. The phosphate linker can also be modified by the replacement of a linking oxygen with nitrogen (bridged phosphoroamidates), sulfur (bridged phosphorothioates) and carbon (bridged methylene-phosphonates).

According to a further preferred embodiment, the artificial nucleic acid comprises an abasic site. As used herein, an ‘abasic site’ is a nucleotide lacking the organic base. In preferred embodiments, the abasic nucleotide further comprises a chemical modification as described herein at the 2′ position of the ribose. Preferably, the 2′ C atom of the ribose is substituted with a substituent selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. Preferred abasic site nucleotides are characterized by the following structures 1A or 1B:

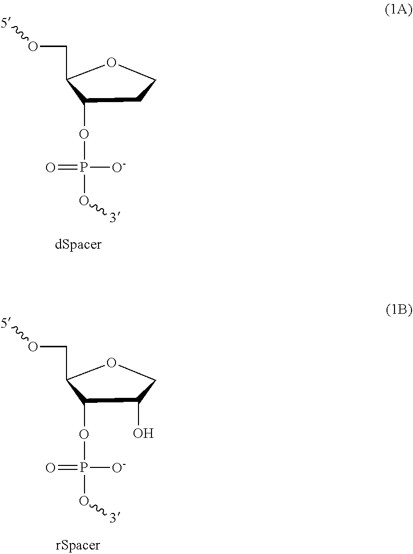

In the context of the present invention, a ‘chemically modified nucleotide’ may therefore also be an abasic site.

According to another embodiment, the artificial nucleic acid molecule can be modified by the addition of a so-called ‘5’ CAP′ structure. A 5′-cap is an entity, typically a modified nucleotide entity, which generally ‘caps’ the 5′-end of a mature mRNA. A 5′-cap may typically be formed by a modified nucleotide, particularly by a derivative of a guanine nucleotide. Preferably, the 5′-cap is linked to the 5′-terminus of the artificial nucleic acid via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′-phosphorothioate, phosphorodithioate, or bridging or non-bridging methylphosphonate moiety. Particularly preferred modified 5′-CAP structures are CAP1 (methylation of the ribose of the adjacent nucleotide of m7G), CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Targeting Sequence

The artificial nucleic acid according to the invention comprises a targeting sequence, which comprises a nucleic acid sequence complementary to a target sequence in the target RNA and wherein the targeting sequence comprises at least one nucleotide, wherein the nucleobase is chemically modified, and/or wherein the targeting sequence comprises at least one backbone modification. In this section, the targeting sequence is described in more detail. However, the description provided in other sections herein, especially with respect to the artificial nucleic acid and with respect to the recruiting moiety, likewise applies to the targeting sequence. In particular, the description of the chemical modifications provided therein also concern the targeting sequence.

According to a preferred embodiment, the targeting sequence comprises at least one chemically modified nucleotide, which is chemically modified at the 2′ position. Preferably, the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro; and/or wherein the chemically modified nucleotide is selected from the group consisting of a locked nucleic acid (LNA) nucleotide, an ethylene bridged nucleic acid (ENA) nucleotide and an (S)-constrained ethyl cEt nucleotide.

Preferably, the targeting sequence of the artificial nucleic acid comprises at least one backbone modification, wherein a nucleotide comprises a modified phosphate group. The modified phosphate group is preferably selected from the group consisting of a phosphorothioate, a phosphoroselenate, a borano phosphate, a borano phosphate ester, a hydrogen phosphonate, a phosphoroamidate, an alkyl phosphonate, an aryl phosphonate and a phosphotriester, most preferably a phosphorothioate.

According to some embodiments, at least about 20%, preferably at least about 40%, more preferably at least about 60%, even more preferably at least about 80%, most preferably at least about 95%, of the nucleotides of the targeting sequence are chemically modified at the 2′ position, preferably by a modification as described herein.

At the position corresponding to the target site (the nucleotide to be edited) in a target RNA, the targeting sequence comprises a cytidine nucleotide or a variant of a cytidine nucleotide, preferably a cytidine ribonucleotide, a deoxycytidine nucleotide, a modified cytidine ribonucleotide, a modified deoxycytidine nucleotide, or an abasic site. In this context, ‘the position corresponding to the target site’ or ‘the position corresponding to the nucleotide to be edited’ refers to the nucleotide position in the targeting sequence that is opposite of said target site, when the target sequence is aligned with a target RNA, preferably by specific base-pairing as described herein. In preferred embodiments, the targeting sequence comprises at the position corresponding to the target site a cytidine or a variant thereof, a deoxycytidine or a variant thereof, or an abasic site, preferably as described herein.

In some embodiments, the target site in the target RNA comprises two or more nucleotides to be edited, wherein these nucleotides are preferably separated from each other by at least one, preferably two, other nucleotides. In these embodiments, the targeting sequence may comprise at each position corresponding to a nucleotide to be edited a nucleotide as described above, preferably a cytidine or a variant thereof, a deoxycytidine or a variant thereof, or an abasic site, preferably as described herein (such as illustrated, for example, by the nucleic acid sequence according to SEQ ID NO: 16).

In a preferred embodiment, at least one, preferably both, of the two nucleotides, which are positioned 5′ or 3′ of the position corresponding to the target site, preferably 5′ or 3′ of the cytidine nucleotide or a variant thereof, the deoxycytidine nucleotide or a variant thereof, or of the abasic site, is chemically modified at the 2′ carbon atom, wherein the 2′ carbon atom is linked to a substituent selected from the group consisting of a halogen, an alkoxy group, a hydrogen, an aryloxy group, an amino group and an aminoalkoxy group, preferably selected from 2′-O-methyl, 2′-O-methoxyethyl, 2′-hydrogen (2′-deoxy) and 2′-fluoro;

and/or
wherein at least one, preferably both, of the two nucleotides, which are positioned 5′ or 3′ of the cytidine nucleotide or a variant thereof, the deoxycytidine nucleotide or a variant thereof, or of the abasic site at the position corresponding to the target site, comprises a modified phosphate group, preferably a phosphorothioate group.

It was surprisingly found that reducing chemical modification of at least one, preferably both, of the two nucleotides surrounding the nucleotide corresponding to the target site (which is preferably a cytidine nucleotide or a variant thereof, a deoxycytidine nucleotide or a variant thereof, or a abasic site) significantly increases the specificity of the RNA editing reaction by reducing off-target editing and preferably also increases the serum stability of the artificial nucleic acid. Prior to the present invention, it was commonly believed in the field that the nucleotide at the position corresponding to the nucleotide to be edited as well as the two nucleotides flanking said nucleotide in the targeting sequence should not be modified. The excellent results obtained by the inventors when using artificial nucleic acids, wherein the nucleotide triplet opposite the target site comprises at least one chemically modified nucleotide as described herein, were thus all the more unexpected.

In this context, it is particularly preferred that the targeting sequence comprises the nucleic acid sequence

3′As*cC*5′,

wherein
As is an adenosine nucleotide or a variant thereof, preferably an adenosine ribonucleotide or a deoxyadenosine nucleotide, further comprising a phosphorothioate group;
c is a cytidine nucleotide or a variant thereof, a deoxycytidine nucleotide or a variant thereof, or an abasic site, at the position corresponding to a nucleotide, preferably an adenosine or a cytidine, more preferably an adenosine, to be edited in the target sequence;
C is a cytidine nucleotide or a variant thereof;
wherein an asterisk (*) indicates a chemical modification of the preceding nucleotide at the 2′ carbon atom with 2′-hydrogen (2′-deoxy), 2′-O-methyl, 2′-O-methoxyethyl or 2′-fluoro.

In some embodiments, it is preferred that the targeting sequence comprises the nucleic acid sequence

3′AcC5′,

A is an adenosine nucleotide or a variant thereof, preferably an adenosine ribonucleotide or a deoxyadenosine nucleotide;
c is a deoxycytidine nucleotide or a modified deoxycytidine nucleotide at the position corresponding to a nucleotide, preferably an adenosine or a cytidine, more preferably an adenosine, to be edited in the target sequence; and
C is a cytidine nucleotide or a variant thereof, preferably a cytidine ribonucleotide, a modified cytidine ribonucleotide, a deoxycytidine nucleotide or a modified deoxycytidine nucleotide, more preferably a deoxycytidine nucleotide or a modified deoxycytidine nucleotide.

According to another embodiment, the targeting sequence comprises the nucleic acid sequence

3′Us*cC*5′,

wherein
Us is an uridine nucleotide or a variant thereof, preferably an uridine ribonucleotide or a deoxyuridine nucleotide, further comprising a phosphorothioate group;
c is a cytidine nucleotide or a variant thereof, a deoxycytidine nucleotide or a variant thereof, or an abasic site, at the position corresponding to a nucleotide, preferably an adenosine or a cytidine, more preferably an adenosine, to be edited in the target sequence;
C is a cytidine nucleotide or a variant thereof;
wherein an asterisk (*) indicates a chemical modification of the preceding nucleotide at the 2′ carbon atom with 2′-hydrogen (2′-deoxy), 2′-O-methyl, 2′-O-methoxyethyl or 2′-fluoro.

It is further preferred that at least two of the five nucleotides at the 3′ terminus of the targeting sequence of the artificial nucleic acid described herein comprise a modified phosphate group, preferably a modified phosphate group as defined herein, more preferably a phosphorothioate group.

In certain embodiments, the nucleotide at the position corresponding to a nucleotide, preferably an adenosine or a cytidine, more preferably an adenosine, to be edited in the target sequence, is an abasic site, preferably an abasic site as described herein. Such an embodiment is particularly preferred, if the deaminase comprises mutations, which reduce the deaminase's activity with respect to natural (physiological) targets (such as an adenosine or a cytidine nucleotide at the target site). Examples of such mutated deaminases include ADAR2 mutants E488Y, E488F or E488W.

Alternatively or in addition the modifications described above, at least two of the five nucleotides at the 3′ terminus of the targeting sequence are preferably LNA nucleotides, ENA nucleotides or (S)-constrained ethyl cEt nucleotides, more preferably LNA nucleotides.

In a preferred embodiment, the targeting sequence of the artificial nucleic acid comprises at least one nucleotide comprising a modified phosphate group, preferably a modified phosphate group as defined herein, more preferably a phosphorothioate nucleotide;

at least one LNA nucleotide; and
at least one nucleotide comprising a substituent at the 2′ carbon atom, wherein the substituent is selected from the group consisting of a halogen, an alkoxy group, a hydrogen (2′-deoxy), an aryloxy group, an amino group and an aminoalkoxy group, preferably selected from 2′-O-methyl, 2′-O-methoxyethyl, 2′-hydrogen (2′-deoxy) and 2′-fluoro.

In certain embodiments, the targeting sequence of the artificial nucleic acid is characterized by a modification pattern according to any one of formulae (Ia), (Ib) or (Ic):

3′NₐCN_{b}5′ (Ia)

wherein
N is a nucleotide or a variant thereof, preferably a ribonucleotide or a variant thereof, a deoxynucleotide or a variant thereof, more preferably a modified ribonucleotide, or a modified deoxynucleotide as described herein;
C is the nucleotide at the position corresponding to the nucleotide to be edited in the target sequence and wherein C is a cytidine nucleotide or a variant thereof, a deoxycytidine nucleotide or a variant thereof, or an abasic site;
a is an integer in a range from 1 to 40, preferably from 6 to 10;
b is an integer in a range from 4 to 40; and
wherein a+b is in a range from 15 to 80;

3′N_{c}Ns_{d}CN_{b}NsₑN_{f}5′ (Ib)

wherein
N is a nucleotide or a variant thereof, preferably a ribonucleotide or a variant thereof, a deoxynucleotide or a variant thereof, more preferably a modified ribonucleotide, or a modified deoxynucleotide as described herein;
C is the nucleotide at the position corresponding to the nucleotide to be edited in the target sequence and wherein C is a cytidine nucleotide or a variant thereof, a deoxycytidine nucleotide or a variant thereof, or an abasic site; Ns is a nucleotide comprising a modified phosphate group, preferably a phosphorothioate group;
c is an integer in a range from 0 to 4;
d is an integer in a range from 1 to 10;
a is an integer in a range from 1 to 26;
b is an integer in a range from 4 to 40;
e is an integer in a range from 0 to 4;
f is an integer in a range from 0 to 4;
wherein a+d+c is in a range from 1 to 40;
wherein b+e+f is in a range from 4 to 40; and
wherein a+d+c+b+e+f is in a range from 15 to 80;

3′N_{c}NI_{g}NₕNIᵢNₐCN_{b}NIⱼNₖNIₗNₘ5′ (Ic)

wherein
N is a nucleotide or a variant thereof, preferably a ribonucleotide or a variant thereof, a deoxynucleotide or a variant thereof, more preferably a modified ribonucleotide, or a modified deoxynucleotide as described herein;
C is the nucleotide at the position corresponding to the nucleotide to be edited in the target sequence and wherein C is a cytidine nucleotide or a variant thereof, a deoxycytidine nucleotide or a variant thereof, or an abasic site; NI is an LNA nucleotide or a modified LNA nucleotide;
c is an integer in a range from 0 to 4, preferably from 1 to 3;
g, i is an integer in a range from 1 to 5;
h is an integer in a range from 1 to 30, preferably from 1 to 5;
a is an integer in a range from 1 to 15;
b is an integer in a range from 4 to 30;
j is an integer in a range from 0 to 5, preferably from 1 to 3;
k is an integer in a range from 4 to 30;
is an integer in a range from 0 to 5, preferably from 1 to 3;
m is an integer in a range from 0 to 3;
wherein c+g+h+i+a is in a range from 1 to 40;
wherein b+j+k+l+m is in a range from 4 to 40; and
wherein c+g+h+i+a+b+j+k+l+m is in a range from 15 to 80.

According to further preferred embodiments the targeting sequence is characterized by a modification pattern selected from any one of the formulae II(a) to II(l):

3′Ns₄N₆CN₇₋₂₉5′; (a)

3′Ns₄N₆₋₁₀CN₉₋₁₂Ns₂5′; (b)

3′Ns₂N₁₁₋₁₅CN₉₋₁₂Ns₂5′; (c)

3′NIs₂Ns₂NIN₆₋₁₀CN₅₋₉NI₂NNs₂5′; (d)

3′NIsNsNIsNsN₆₋₁₀CN₄₋₈NINNINNs₂5′; (e)

3′NsNIsNsNIsN₆₋₁₀CN₃₋₇NINNIN₂Ns₂5′; (f)

3′Ns₂NNINNIN₆₋₁₀CN₄₋₈NINNINNs₂5′, (g)

3′NsNIsNs₂NIN₅CN₅NIN₁₋₂₃5′; (h)

3′NIsNsNIsNsN₈CN₆NIN₁₋₂₃5′ (i)

3′NsNIsNs₂NIN₅CN₅NIN₂₀NI₂5′; (j)

3′NIsNsNIsNsN₈CN₆NIN₂₀NI₂5′; and (k)

3′Ns₄N₆CN₉Ns₂5′, (l)

wherein
N is a nucleotide or a variant thereof, preferably a ribonucleotide or a variant thereof, a deoxynucleotide or a variant thereof, more preferably a modified ribonucleotide, or a modified deoxynucleotide as described herein;
Ns is a nucleotide comprising a modified phosphate group, preferably a phosphorothioate group;
NI is an LNA nucleotide or a modified LNA nucleotide;
NIs is an LNA nucleotide or a modified LNA nucleotide, further comprising a modified phosphate group, preferably a phosphorothioate group;
C is the nucleotide at the position corresponding to the nucleotide to be edited in the target sequence and wherein C is a cytidine nucleotide or a variant thereof, a deoxycytidine nucleotide or a variant thereof, or an abasic site.

The formulae (Ia), (Ib), (Ic) as well as formulae II(a)-(I) describe a modification pattern of the targeting sequence of the artificial nucleic acid described herein. A modification pattern as used herein refers to the presence (or absence, respectively) of certain modifications as indicated in the formulae at certain positions in the targeting sequence. The respective position can be derived from said formulae, in particular the relative position of said modifications with regard to the nucleotide at the position corresponding to the nucleotide to be edited in the target RNA, preferably a cytidine or a variant thereof, a deoxycytidine or a variant thereof or an abasic site. The formulae above define a modification pattern, which applies to a variety of nucleic acid sequences, which comprise the nucleotides defined in the formulae. The individual nucleic acid sequence of a targeting sequence of an artificial nucleic acid for editing a given target RNA always depends on that specific target RNA and the target site. Nevertheless, the modification patterns identified herein are applicable independent from the specific nucleic acid sequence and define the number and the type of modification and their relative position.

In this context, it is noted that the subscript numbers (and variables) used in said formulae indicate the number of the specific type of nucleotide, that is present in the targeting sequence. For instance, ‘N₁₁₋₁₃’ that the targeting sequence comprises (at that position) from 11 to 13 (i.e. 11, 12 or 13) nucleotides as defined by the formula. Hence, that exemplary modification pattern applies to nucleic acid sequences comprising at that position 11, 12 or 13 nucleotides of that type.

According to some embodiments, the targeting sequence of the artificial nucleic acid as described herein is characterized by a modification pattern, wherein,

with the exception of the cytidine nucleotide or the variant thereof, the deoxycytidine nucleotide or a variant thereof, preferably the deoxycytidine nucleotide, or the abasic site, at the position corresponding to the nucleotide to be edited in the target sequence,
with the exception of LNA nucleotides, and
optionally with the exception of at least one of the two nucleotides, which are positioned 5′ or 3′ to the nucleotide at the position corresponding to the nucleotide to be edited in the target sequence,
all nucleotides are chemically modified at the 2′ carbon atom, which is linked to a substituent selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro.

In certain embodiments, the targeting sequence of the artificial nucleic acid comprises or consists of a nucleic acid sequence selected from the group consisting of

(SEQ ID NO: 1)
5′ U*U*C*A*C*U* UcA G*U*G*U*As*Us*Gs*Cs*C* 3′;
(SEQ ID NO: 2)
5′ U*U*C*A*C*U* UcA G*U*G*U*As*Us*Gs*Cs*C* 3′;
(SEQ ID NO: 3)
5′ A*C*C*U*C*C* AcU C*A*G*U*Gs*Us*Gs*As*U* 3′;
(SEQ ID NO: 4)
5′ U*U*U*C*C*U* CcA C*U*G*U*Us*Gs*Cs*As*A* 3′;
(SEQ ID NO: 5)
5′ U*G*U*G*U*A* UcU U*G*C*U*Gs*Us*Gs*As*G* 3′;
(SEQ ID NO: 6)
5′ G*A*G*G*U*C* CcU G*G*G*G*Gs*Cs*Gs*Cs*U* 3′;
(SEQ ID NO: 7)
5′ G*A*U*C*U*U* CcU G*A*U*G*Gs*Cs*Cs*As*C* 3′;
(SEQ ID NO: 8)
5′ A*G*C*C*A*C* AcA C*U*C*C*Gs*Us*Cs*As*G* 3′;
(SEQ ID NO: 9)
5′ G*A*U*U*U*U* CcU G*A*U*A*Gs*Cs*Us*As*C* 3′;
(SEQ ID NO: 10)
5′ G*G*C*C*A*C* AcA U*U*C*U*Gs*Us*Cs*As*G* 3′;
(SEQ ID NO: 11)
5′ G*A*U*C*U*U* CcU G*A*U*G*Gs*Cs*Cs*As*C* 3′;
(SEQ ID NO: 12)
5′ G*G*C*C*A*C* AcA C*U*C*C*Gs*Us*Cs*As*G* 3′;
(SEQ ID NO: 13)
5′ G*A*U*U*U*U* CcU G*A*U*A*Gs*Cs*As*As*C* 3′;
(SEQ ID NO: 14)
5′ G*G*C*U*A*C* GcA C*U*C*U*Gs*Us*Cs*As*A* 3′;
(SEQ ID NO: 15)
5′ A*G*G*C*C*G* CcG U*C*G*U*Gs*Gs*Cs*Gs*G* 3′;
(SEQ ID NO: 16)
5′ C*C*G*C*U*C* CcU CcU C*A*G*C*Cs*Cs*Gs*Us*C* 3′;
(SEQ ID NO: 17)
5′ A*C*G*C*C*A* CcA G*C*U*C*Cs*As*As*Cs*U* 3′;
(SEQ ID NO: 18)
5′ G*U*C*U*C*A* CcA A*U*U*G*Cs*Us*Cs*Us*C* 3′;
(SEQ ID NO: 19)
5′ G*A*A*A*U*A* CcA U*C*A*G*As*Us*Us*Us*G* 3;
(SEQ ID NO: 20)
5′ A*A*U*U*A*G* CcU U*C*U*G*Gs*Cs*Cs*As*U* 3′;
(SEQ ID NO: 21)
5′ G*A*U*C*A*G* CcU C*C*U*G*Gs*Cs*Cs*As*U* 3′;
(SEQ ID NO: 22)
5′ G*A*U*C*A*G* CcU U*C*U*G*Gs*Cs*Cs*As*U* 3′;
(SEQ ID NO: 23)
5′ G*A*U*C*A*G* CcU U*C*U*G*Gs*Cs*Cs*As*U* 3′;
(SEQ ID NO: 24)
5′ C*A*C*U*G*C* CcA G*G*C*A*Us*Cs*As*Gs*C* 3′;
(SEQ ID NO: 25)
5′ C*A*C*U*G*C* CcG G*G*C*A*Us*Cs*As*Gs*C* 3′;
(SEQ ID NO: 26)
5′ U*C*C*G*C*C* CcG A*U*C*C*As*Cs*Gs*As*U* 3′;
(SEQ ID NO: 27)
5′ C*C*U*U*U*C* UcG U*C*G*A*Us*Gs*Gs*Us*C* 3′;
(SEQ ID NO: 28)
5′ C*C*U*U*U*C* U*cG U*C*G*A*Us*Gs*Gs*Us*C* 3′;
(SEQ ID NO: 29)
5′ C*U*U*G*A*U* AcA U*C*C*A*Gs*Us*Us*Cs*C* 3′;
(SEQ ID NO: 30)
5′ U*U*U*C*A*G* GcA U*U*U*C*Cs*Us*Cs*Cs*G* 3′;
(SEQ ID NO: 31)
5′ C*U*U*C*A*G* GcA U*G*G*G*Gs*Cs*As*Gs*C* 3′;
(SEQ ID NO: 32)
5′ A*G*G*A*A*C* AcA A*C*C*U*Us*Us*Gs*Us*C* 3′;
(SEQ ID NO: 33)
5′ U*U*U*C*A*C* AcA U*C*C*A*Us*Cs*As*As*C* 3′;
(SEQ ID NO: 34)
5′ C*U*U*C*A*C* GcA U*C*C*A*Us*Cs*As*As*C* 3′;
(SEQ ID NO: 35)
5′ U*G*G*G*A*C* AcA A*C*C*C*Cs*Us*Gs*Cs*C* 3′;
(SEQ ID NO: 36)
5′ C*G*A*C*U*C* CcU C*U*G*G*As*Us*Gs*Us*U* 3′;
(SEQ ID NO: 37)
5′ C*G*A*C*U*C* UcU C*U*G*G*As*Us*Gs*Us*U* 3′;

or a fragment or variant of any of these sequences,
wherein
A is an adenosine nucleotide or a variant thereof, preferably an adenosine ribonucleotide, an adenosine deoxynucleotide, a modified adenosine ribonucleotide or a modified adenosine deoxynucleotide;
C is a cytidine nucleotide or a variant thereof, preferably a cytidine ribonucleotide, a cytidine deoxynucleotide, a modified cytidine ribonucleotide or a modified cytidine deoxynucleotide;
G is a guanosine nucleotide or a variant thereof, preferably a guanosine ribonucleotide, a guanosine deoxynucleotide, a modified guanosine ribonucleotide or a modified guanosine deoxynucleotide;
U is an uridine nucleotide or a variant thereof, preferably an uridine ribonucleotide, an uridine deoxynucleotide, a modified uridine ribonucleotide or a modified uridine deoxynucleotide;
As, Cs, Gs and Us are nucleotides, preferably ribonucleotides or deoxynucleotides as defined above, further comprising a phosphorothioate group;
wherein an asterisk (*) indicates a chemical modification of the preceding nucleotide at the 2′ carbon atom, preferably with 2′-hydrogen (2′-deoxy), 2′-O-methyl, 2′-O-methoxyethyl or 2′-fluoro; and
wherein a lower case letter c indicates the position corresponding to a nucleotide, preferably an adenosine or a cytidine, more preferably an adenosine, to be edited in the target sequence and wherein c represents a cytidine nucleotide or a variant thereof, a deoxycytidine nucleotide or a variant thereof, or an abasic site.

In the context of the present invention, a ‘variant’ of a nucleic acid sequence or of an amino acid sequence is at least 40%, preferably at least 50%, more preferably at least 60%, more preferably at least 70%, even more preferably at least 80%, even more preferably at least 90%, most preferably at least 95% identical to the sequence, the variant is derived from. Preferably, the variant is a functional variant.

As used herein, a ‘fragment’ of a nucleic acid sequence or of an amino acid sequence consists of a continuous stretch of nucleotides or amino acid residues corresponding to a continuous stretch of nucleotides or amino acid residues in the full-length sequence, which represents at least 5%, 10%, 20%, preferably at least 30%, more preferably at least 40%, more preferably at least 50%, even more preferably at least 60%, even more preferably at least 70%, even more preferably at least 80%, and most preferably at least 90% of the full-length sequence, the fragment is derived from. Such a fragment, in the sense of the present invention, is preferably a functional fragment.

According to some embodiments, the targeting sequence of the artificial nucleic acid comprises at the position corresponding to a nucleotide to be edited in the target sequence a cytidine nucleotide or a variant thereof, a deoxycytidine nucleotide or a variant thereof, or an abasic site,

wherein the nucleotide or a variant thereof, which is positioned 5′ of the position corresponding to the nucleotide to be edited, is a pyrimidine nucleotide, preferably a pyrimidine ribonucleotide or a pyrimidine deoxynucleotide, and wherein said pyrimidine nucleotide comprises a nucleobase, which is chemically modified at the 2′ position, preferably by 2′-hydrogen (2′-deoxy), 2′-O-methyl, 2′-O-methoxyethyl or 2′-fluoro.

In an alternative embodiment, the targeting sequence of the artificial nucleic acid comprises at the position corresponding to a nucleotide to be edited in the target sequence a cytidine nucleotide or a variant thereof, a deoxycytidine or a variant thereof, preferably a deoxycytidine nucleotide, or an abasic site,

wherein at least one, preferably both, of the two nucleotides or a variant thereof, which are positioned 5′ or 3′ of the position corresponding to the nucleotide to be edited, are chemically modified at the 2′ carbon atom, which is linked to a substituent selected from the group consisting of a halogen, an alkoxy group, a hydrogen, an aryloxy group, an amino group and an aminoalkoxy group, preferably selected from 2′-O-methyl, 2′-O-methoxyethyl, 2′-hydrogen and 2′-fluoro;
and/or
wherein at least one, preferably both, of the two nucleotides or a variant thereof, which are positioned 5′ or 3′ of the position corresponding to the nucleotide to be edited, comprises a modified phosphate group, preferably a modified phosphate group as described herein, more preferably a phosphorothioate group.

Recruiting Moiety with Coupling Agent

According to some embodiments of the present invention, the artificial nucleic acid comprises the targeting sequence as described herein and further comprises a recruiting moiety comprising at least one coupling agent. Said coupling agent is capable of recruiting a deaminase, which comprises a moiety that binds to said coupling agent. As mentioned above, the recruiting moiety comprises or consists of a coupling agent, which recruits a deaminase and which is typically covalently linked to the targeting sequence. More preferably, the recruiting moiety consists of a coupling agent as described herein, which is linked, preferably covalently, to the 5′-terminus or to the 3′-terminus of the targeting sequence. Alternatively, the coupling agent may also be linked, preferably covalently, to an internal nucleotide (i.e. not a 5′- or 3′-terminal nucleotide) of the targeting sequence, for example via linkage to a nucleotide variant or a modified nucleotide, preferably as described herein, such as amino-thymidine.

The coupling agent, which recruits a deaminase is typically covalently linked to the targeting sequence. Preferably, the coupling agent is linked to the 5′-terminus or to the 3′-terminus of the targeting sequence. The coupling agent may alternatively also be linked to an internal nucleotide (i.e. not a 5′- or 3′-terminal nucleotide) of the targeting sequence, for example via linkage to a nucleotide variant or a modified nucleotide, preferably as described herein, such as amino-thymidine.

In a preferred embodiment, the coupling agent is selected from the group consisting of O6-benzylguanine, O2-benzylcytosine, chloroalkane, 1×BG, 2×BG, 4×BG, and a variant of any of these. According to a particularly preferred embodiment, the coupling agent is a branched molecule, such as 2×BG or 4×BG, each of which is preferably capable of recruiting a deaminase molecule, thus preferably amplifying the editing reaction. Exemplary structures of suitable branched coupling agents are depicted below:

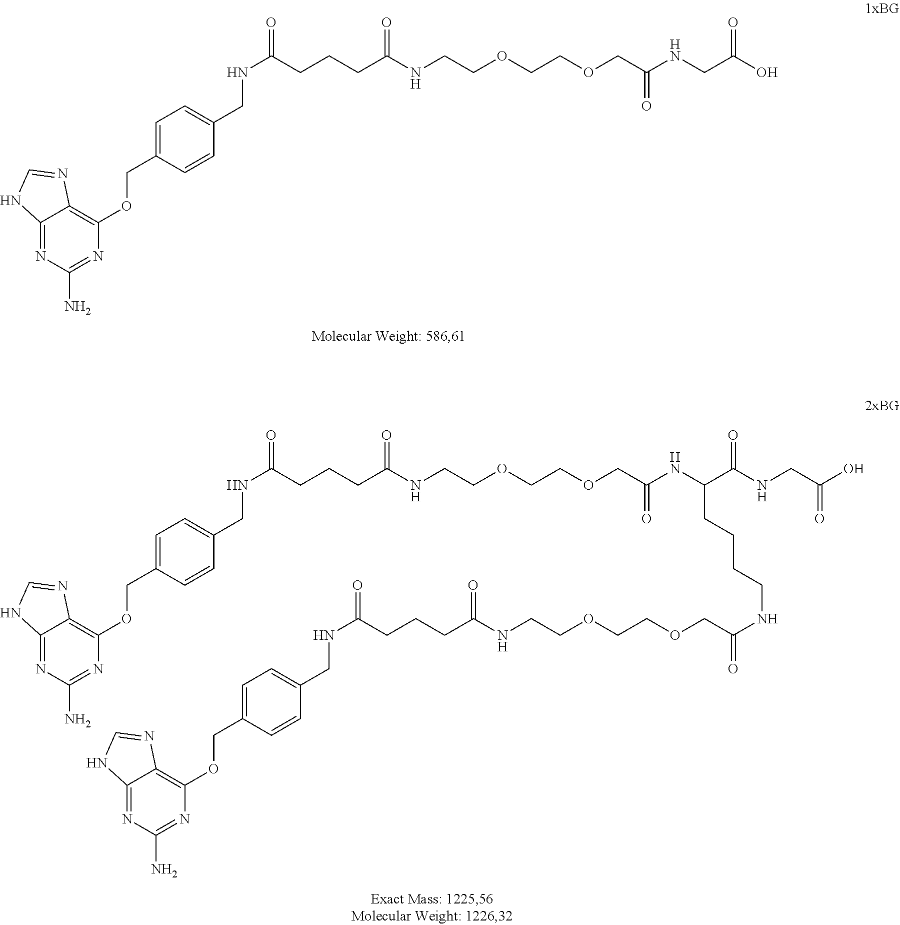 [SMILES: Nc1nc(OCc2ccc(CNC(=O)CCCC(=O)NCCOCCOCC(=O)NCC(=O)O)cc2)c2nc[nH]c2n1 ; Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)NCC(=O)O)cc2)c2nc[nH]c2n1]

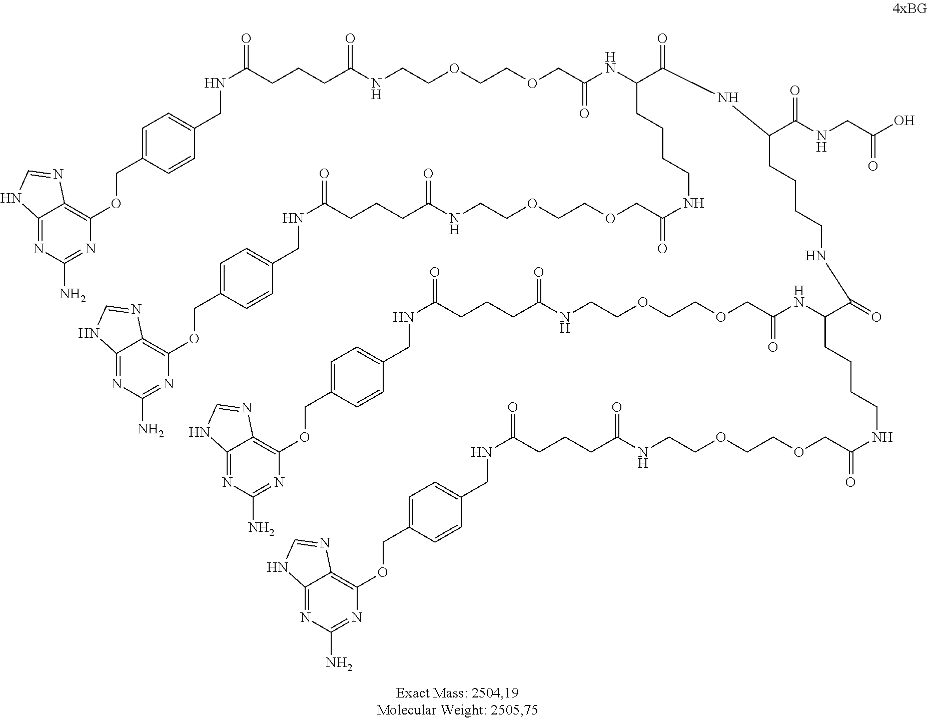 [SMILES: Nc1nc(OCc2ccc(CNC(=O)CCCC(=O)NCCOCCOCC(=O)NCCCCC(NC(=O)COCCOCCNC(=O)CCCC(=O)NCc3ccc(COc4nc(N)nc5[nH]cnc45)cc3)C(=O)NCCCCC(NC(=O)C(CCCCNC(=O)COCCOCCNC(=O)CCCC(=O)NCc3ccc(COc4nc(N)nc5[nH]cnc45)cc3)NC(=O)COCCOCCNC(=O)CCCC(=O)NCc3ccc(COc4nc(N)nc5[nH]cnc45)cc3)C(=O)NCC(=O)O)cc2)c2nc[nH]c2n1]

The coupling agent is preferably capable of specifically binding to a moiety in a deaminase. Said moiety in a deaminase is preferably a tag, which is linked to a deaminase as described herein, preferably an adenosine deaminase or a cytidine deaminase as described herein. More preferably, said tag is selected from the group consisting of a SNAP-tag, a CLIP-tag, a HaloTag, and a fragment or variant of any one of these. Accordingly, the deaminases bound by the coupling agent in these embodiments are preferably artificial versions of endogenous deaminases, preferably of a deaminase as described herein. Preferably, the deaminase is selected from the group consisting of SNAP-ADAR1, SNAP-ADAR2, Apobec1-SNAP, SNAPf-ADAR1, SNAPf-ADAR2, Apobec1-SNAPf, Halo-ADAR1, Halo-ADAR2, Apobec1-Halo, Clip-ADAR1, Clip-ADAR2, Clipf-ADAR1, Clipf-ADAR2, Apobec1-Clip and Apobec1-Clipf, preferably as described herein, or a fragment or variant of any of these, wherein the deaminase is preferably derived from human or mouse. More preferably, the deaminase is selected from the group consisting of SNAP-ADAR1, SNAP-ADAR2, SNAPf-ADAR1, SNAPf-ADAR2, Halo-ADAR1, Halo-ADAR2, Clip-ADAR1, Clip-ADAR2, Clipf-ADAR1 and Clipf-ADAR2, or a fragment or variant of any of these, wherein the deaminase is derived from human. According to another embodiment, the deaminase is selected from the group consisting of mApobec1-SNAP, mApobec1-SNAPf, mApobec1-Halo,m Apobec1-Clip and mApobec1-Clipf, or a fragment or variant of any of these, wherein the deaminase is derived from mouse. In a particularly preferred embodiment, the deaminase is a hyperactive mutant of any of the deaminases mentioned herein, preferably a hyperactive Q mutant, more preferably a hyperactive Q mutant of an ADAR1 deaminase, an ADAR2 deaminase (e.g. human ADAR1p150, E1008Q; human ADAR1p110, E713Q; human ADAR2, E488Q) or a tagged version thereof, most preferably as described herein, or a fragment or variant of any of these.

Tagged deaminases, preferably as described herein, (e.g. SNAP-, SNAPf-, Clip-, Clipf-, Halo-tagged deaminases or fragments or variants thereof) are preferably overexpressed for RNA editing, for example by transient transfection of a cell with a vector encoding said tagged deaminase or by stable expression in a transgenic cell, tissue or organism.

According to a preferred embodiment, the recruiting moiety comprises or consists of a coupling agent selected from the group consisting of 06-benzylguanine, 1×BG, 2×BG, 4×BG and a variant of any one of these. In this embodiment, the artificial nucleic acid is used in presence of a deaminase, preferably an adenosine or cytidine deaminase, more preferably as described herein, wherein the deaminase comprises a SNAP-tag or a variant thereof. In an alternative embodiment, the recruiting moiety comprises or consists of a chloroalkane and the deaminase, preferably an adenosine or cytidine deaminase, more preferably as described herein, comprises a HaloTag or a variant thereof. According to a further embodiment, the recruiting moiety comprises O2-benzylcytosine or a variant thereof and the deaminase, preferably an adenosine or cytidine deaminase, more preferably as described herein, comprises a Clip-tag or a variant thereof.

In certain embodiments, the artificial nucleic acid as described herein comprises the targeting sequence as described herein at least two or more recruiting moieties, wherein each recruiting moiety comprises or consists of a coupling agent as described herein and wherein each recruiting moiety preferably recruits a deaminase molecule, thus preferably amplifying the editing reaction. Each of these recruiting moieties preferably comprises—independently from the other recruiting moieties—a coupling agent selected from the group consisting of O6-benzylguanine, O2-benzylcytosine, chloroalkane, 1×BG, 2×BG, 4×BG, and a variant of any of these. Preferably, the artificial nucleic acid comprises at least two recruiting moieties, wherein each recruiting moiety comprises the same or a different coupling agent. Schematic structures of embodiments comprising more than one recruiting moiety and/or comprising branched coupling agents are illustrated by FIG. 11 herein.

Recruiting Moiety with Nucleic Acid Recruiting Motif

In preferred embodiments of the present invention, the artificial nuclei acid comprises a targeting sequence as described herein and a recruiting moiety comprising or consisting of a nucleic acid sequence capable of specifically binding to the deaminase, preferably an adenosine or cytidine deaminase. Preferably, the nucleic acid sequence capable of specifically binding to the deaminase specifically binds to a double-stranded (ds) RNA binding domain of a deaminase, preferably as described herein. Advantageously, the recruiting moiety comprising or consisting of a nucleic acid sequence capable of specifically binding to a deaminase also binds to endogenous deaminases. The artificial nucleic acid according to the invention thus promotes site-directed RNA editing employing an endogenous (or heterologously expressed) deaminase.

Preferably, the recruiting moiety comprises or consists of a nucleic acid sequence capable of specifically binding to a deaminase, wherein the nucleic acid sequence is preferably linked covalently either to the 5′ terminus or to the 3′ terminus of the targeting sequence, more preferably to the 5′ terminus of the targeting sequence. In certain embodiments, the artificial nucleic acid comprises a targeting sequence as described herein and at least two recruiting moieties as described herein.

In some embodiments, the recruiting moiety comprises or consists of a nucleic acid sequence that is capable of intramolecular base pairing. The recruiting moiety preferably comprises or consists of a nucleic acid sequence that is capable of forming a stem-loop structure. In certain embodiments, said stem-loop structure comprises or consists of a double-helical stem comprising at least two mismatches. In a preferred embodiment, the stem loop structure comprises a loop consisting of from 3 to 8, preferably from 4 to 6, more preferably 5, nucleotides. The loop preferably comprises or consists of the nucleic acid sequence GCUAA or GCUCA.

According to preferred embodiments, the recruiting moiety of the artificial nucleic acid comprises or consists of a nucleic acid sequence comprises at least one chemical modification as described herein. In particular, the recruiting moiety of the artificial nucleic acid preferably comprises or consists of a nucleic acid sequence comprises at least one nucleotide, wherein the nucleobase is chemically modified, and/or wherein the nucleic acid sequence comprises at least one backbone modification. The chemical modifications described herein in the respective section and further with regard to the artificial nucleic acid in general and the targeting sequence are also applicable to the recruiting moiety.

In some embodiments, the at least one chemically modified nucleotide is chemically modified at the 2′ position. Preferably, the chemically modified base comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro. According to an alternative embodiment, the chemically modified nucleotide is a locked nucleic acid (LNA) nucleotide, an ethylene bridged nucleic acid (ENA) nucleotide or an (S)-constrained ethyl cEt nucleotide.

In preferred embodiments, the artificial nucleic acid comprises a recruiting moiety comprising a nucleic acid sequence as described herein, wherein the recruiting moiety comprises at least one chemically modified nucleotide, wherein the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro; and/or

wherein the chemically modified nucleotide is a locked nucleic acid (LNA) nucleotide, an ethylene bridged nucleic acid (ENA) nucleotide or an (S)-constrained ethyl cEt nucleotide.

Preferably, the recruiting moiety of the artificial nucleic acid comprises at least one backbone modification, wherein a nucleotide comprises a modified phosphate group. The modified phosphate group is preferably selected from the group consisting of a phosphorothioate, a phosphoroselenate, a borano phosphate, a borano phosphate ester, a hydrogen phosphonate, a phosphoroamidate, an alkyl phosphonate, an aryl phosphonate and a phosphotriester, most preferably a phosphorothioate.

In some embodiments, at least about 20%, preferably at least about 40%, more preferably at least about 60%, even more preferably at least about 80%, most preferably at least about 95%, of the nucleotides of the nucleic acid sequence of the recruiting moiety are chemically modified at the 2′ position, preferably by a modification as described herein.

Preferably, the recruiting moiety comprises a nucleic acid sequence, wherein at least of two of the five nucleotides at the 5′ terminus of the nucleic acid sequence comprise a phosphorothioate group.

According to some embodiments, the recruiting moiety comprises a nucleic acid sequence, wherein at least of two of the five nucleotides at the 5′ terminus of the nucleic acid sequence are LNA nucleotides, ENA nucleotides or (S)-constrained ethyl cEt nucleotides.

In a preferred embodiment of the invention, the recruiting moiety comprises a nucleic acid sequence, wherein

at least one nucleotide comprises a modified phosphate group, preferably a phosphorothioate group;
at least one LNA nucleotide, ENA nucleotide or (S)-constrained ethyl cEt nucleotide; and
at least one nucleotide comprising a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl or 2′-fluoro.

According to a particularly preferred embodiment, the recruiting moiety comprises or consists of a nucleic acid sequence selected from the group consisting of

(a)
(SEQ ID NO: 38)
5′ GGUGUCGAG-Nₐ-AGA-N_{c}-GAGAACAAUAU-GCU A/C A-
AUGUUGUUCUC-N_{d}-UCU-N_{b}-CUCGACACC 3′;
(b)
(SEQ ID NO: 39)
5′ GsGsUGUCGAG-Nₐ-AGA-N_{c}-GAGAACAAUAU-GCU A/C A-
AUGUUGUUCUC-N_{d}-UCU-N_{b}-CUCGACACC 3′; and
(c)
(SEQ ID NO: 40)
5′ GslGslUGUCGAG-Nₐ-AGA-N_{c}-GAGAACAAUAU-GCU A/C A-
AUGUUGUUCUC-N_{d}-UCU-N_{b}-CUCGACACC 3′;

or a fragment or variant of any of these;
wherein
Nₐ and N_{b} form a mismatch, preferably wherein Nₐ is adenosine and N_{b} is cytidine;
N_{c} and N_{d} form a mismatch, preferably wherein N_{c} and N_{d} are guanosine;
Gs is a guanosine comprising a phosphorothioate group; and
GsI is an LNA guanosine comprising a phosphorothioate group.

According to an alternative embodiment, the recruiting moiety comprises or consists of a nucleic acid sequence derived from VA (viral associated) RNA I, or a fragment or variant thereof. VA RNA I is an RNA derived from adenovirus and is known to the skilled person. In a preferred embodiment, the recruiting moiety of the artificial nucleic acid comprises the nucleic acid sequence

(SEQ ID NO: 41)
GCACACCTGGGTTCGACACGCGGGCGGTAACCGCATGGATCA
CGGCGGACGGCCGGATTCGGGGTTCGAACCCCGGTCGTCCGC
CATGATACCCTTGC,

or a fragment or variant thereof.

In a preferred embodiment, the recruiting moiety comprises a nucleic acid sequence according to any one of SEQ ID NO: 38 to 41, or a fragment or variant of any of these sequences, wherein at least one nucleotide, preferably at least 40%, 50%, 60%, 70%, 80%, 90%, 95%, 99% or 100% of the nucleotides, comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen (2′-deoxy), 2′-O-methyl, 2′-O-methoxyethyl and 2′-fluoro.

According to a particularly preferred embodiment, the recruiting moiety comprises a nucleic acid sequence selected from the group consisting of

(a)
(SEQ ID NO: 42)
5′ G*G*U*GU*C*GAG-Nₐ-AGA-N_{c}-GAGAAC′AAU*AU*-
GC*U* A/C A-AU*GU*U*GU*U*C*U*C*-N_{d}-U*C*U*-
N_{b}*-C*U*C*GAC*AC*C* 3′;
(b)
(SEQ ID NO: 43)
5′ Gs*Gs*U*GU*C*GAG-Nₐ-AGA-N_{c}-GAGAAC*AAU*
AU*-GC*U* A/C A-AU*GU*U*GU*U*C*U*C*-N_{d}-U*
C*U*-N_{b}*-C*U*C*GAC*AC*C* 3′;
and
(c)
(SEQ ID NO: 44)
5′ Gsl*Gsl*U*GU*C*GAG-Nₐ-AGA-Nc-GAGAAC*AAU*
AU*-GC*U* A/C A-AU*GU*U*GU*U*C*U*C*-N_{d}-U*C*
U*-N_{b}*-C*U*C*GAC*AC*C* 3′;

or a fragment or variant of any of these sequences;
wherein
Nₐ and N_{b} form a mismatch, preferably wherein Nₐ is adenosine and N_{b} is cytidine;
N_{c} and N_{d} form a mismatch, preferably wherein N_{c} and N_{d} are guanosine;
Gs is a guanosine comprising a phosphorothioate group;
GsI is an LNA guanosine comprising a phosphorothioate group; and wherein an asterisk (*) indicates a modification of the nucleotide at the 2′ carbon atom, preferably with 2′-hydrogen (2′-deoxy), 2′-O-methyl, 2′-O-methoxyethyl or 2′-fluoro.

In certain embodiments, it is preferred that the artificial nucleic acid according described herein comprises in 5′ to 3′ direction the recruiting moiety described herein and the targeting sequence described herein.

A further aspect of the present invention concerns an artificial nucleic acid for site-directed editing of a target RNA, the artificial nucleic acid comprising

a) a targeting sequence, which comprises or consists of a nucleic acid sequence complementary or partially complementary to a target sequence in the target RNA,
and
b) a recruiting moiety for recruiting a deaminase, wherein the recruiting moiety comprises or consists of a nucleic acid sequence capable of specifically binding to the deaminase, preferably an adenosine or cytidine deaminase.

According to that aspect, the recruiting moiety is preferably as defined herein under the section ‘Recruiting moiety with nucleic acid recruiting motif’. In preferred embodiments of this aspect of the invention, the targeting sequence is chemically modified, preferably as described herein. In certain embodiments of this aspect, the targeting sequence is not chemically modified. In a particularly preferred embodiment, the artificial nucleic acid is synthesized in a cell, preferably a cell as described herein, more preferably by transcription from a vector, preferably from a vector as described herein. According to a particularly preferred embodiment of this aspect of the present invention, the artificial nucleic acid comprises a recruiting moiety comprising or consisting of a nucleic acid sequence according to any one of SEQ ID NO: 38 to 41, or a fragment or variant thereof.

Deaminase

The artificial nucleic acid is suitable for site-directed editing of an RNA by a deaminase, wherein the deaminase is preferably an adenosine deaminase or a fragment or variant thereof, preferably an ADAR (adenosine deaminase acting on dsRNA) enzyme or a fragment or variant thereof, more preferably selected from the group consisting of ADAR1, ADAR2 and a fragment or variant thereof, even more preferably a peptide or protein comprising an adenosine deaminase domain; or

a cytidine deaminase or a fragment or variant thereof, preferably Apobec1 or a fragment or variant thereof, more preferably a peptide or protein comprising a cytidine deaminase domain. The term ‘deaminase’ as used herein refers to any peptide, protein or protein domain, which is capable of catalyzing the deamination of a nucleotide or a variant thereof in a target RNA, in particular the deamination of adenosine or cytidine. The term thus not only refers to full-length and wild type deaminases, such as ADAR1, ADAR2 or Apobec1, but also to a fragment or variant of a deaminase, preferably a functional fragment or a functional variant. In particular, the term also refers to mutants and variants of a deaminase, such as mutants of ADAR1, ADAR2 or Apobec1, preferably as described herein. Furthermore, the term deaminase as used herein also comprises any deaminase fusion protein (e.g. based on Cas9 and Cas13). In the context of the present invention, the term ‘deaminase’ also refers to tagged variants of a deaminase, such as a deaminase selected from the group consisting of SNAP-ADAR1, SNAP-ADAR2, Apobec1-SNAP, SNAPf-ADAR1, SNAPf-ADAR2, Apobec1-SNAPf, Halo-ADAR1, Halo-ADAR2, Apobec1-Halo, Clip-ADAR1, Clip-ADAR2, Clipf-ADAR1, Clipf-ADAR2, Apobec1-Clip and Apobec1-Clipf, preferably as described herein, or a fragment or variant of any of these, wherein the deaminase is preferably derived from human or mouse.

In a preferred embodiment, the deaminase is an adenosine deaminase (such as ADAR1, preferably ADAR1p150 or ADAR1p110, or ADAR2), preferably a eukaryotic adenosine deaminase, more preferably a vertebrate adenosine deaminase, even more preferably a mammalian adenosine deaminase, most preferably a human adenosine deaminase, such as hADAR1 or hADAR2, or a fragment or variant of any of these. In a particularly preferred embodiment, the deaminase is a tagged adenosine deaminase, preferably as described herein, or a fragment or variant thereof. More preferably, the a deaminase as used herein is selected from the group consisting of SNAP-ADAR1, SNAP-ADAR2, SNAPf-ADAR1, SNAPf-ADAR2, Halo-ADAR1, Halo-ADAR2, Clip-ADAR1, Clip-ADAR2, Clipf-ADAR1 and Clipf-ADAR2, or a fragment or variant of any of these, wherein the deaminase is derived from human.

According to an alternative embodiment, the deaminase is a cytidine deaminase (such as Apobec1, preferably human Apobec1 or murine Apobec1 (mApobec1)), preferably a eukaryotic cytidine deaminase, more preferably a vertebrate cytidine deaminase, even more preferably a mammalian cytidine deaminase, most preferably a murine or human cytidine deaminase, or a fragment or variant of any of these. In a particularly preferred embodiment, the deaminase is a tagged cytidine deaminase, preferably as described herein, or a fragment or variant thereof. According to a preferred embodiment, the deaminase is selected from the group consisting of mApobec1-SNAP, mApobec1-SNAPf, mApobec1-Halo,mApobec1-Clip and mApobec1-Clipf, or a fragment or variant of any of these, wherein the deaminase is derived from mouse.

In preferred embodiments, the deaminase is an endogenous deaminase, or a fragment or variant thereof, preferably as described herein. The artificial nucleic acid comprising a recruiting moiety with nucleic acid recruiting motif (see respective section herein) is preferably used in connection with an endogenous deaminase, or a fragment or variant thereof.

In a particularly preferred embodiment, the deaminase is a hyperactive mutant of any of the deaminases mentioned herein, preferably a hyperactive Q mutant, more preferably a hyperactive Q mutant of an ADAR1 deaminase, an ADAR2 deaminase (e.g. human ADAR1p150, E1008Q; human ADAR1p110, E713Q; human ADAR2, E488Q) or a tagged version thereof, most preferably as described herein, or a fragment or variant of any of these.

A tagged deaminase, preferably as described herein, is preferably used in connection with the artificial nucleic acid according to the invention, wherein the recruiting moiety comprises at least one coupling agent capable of recruiting a deaminase comprising a moiety that binds to said coupling agent (see also section ‘Recruiting moiety with coupling agent’).

In the following particularly preferred deaminases as used herein are described as examples:

hADAR1 p150:
nucleic acid sequence:
(SEQ ID NO: 45)
ATGGCCGAGATCAAGGAGAAAATCTGCGACTATCT
CTTCAATGTGTCTGACTCCTCTGCCCTGAATTTGG
CTAAAAATATTGGCCFTACCAAGGCCCGAGATATA
AATGCTGTGCTAATTGACATGGAAAGGCAGGGGGA
TGTCTATAGACAAGGGACAACCCCTCCCATATGGC
ATTTGACAGACAAGAAGCGAGAGAGGATGCAAATC
AAGAGAAATACGAACAGTGTTCCTGAAACCGCTCC
AGCTGCAATCCCTGAGACCAAAAGAAACGCAGAGT
TCCTCACCTGTAATATACCCACATCAAATGCCTCA
AATAACATGGTAACCACAGAAAAAGTGGAGAATGG
GCAGGAACCTGTCATAAAGTTAGAAAACAGGCAAG
AGGCCAGACCAGAACCAGCAAGACTGAAACCACCT
GTTCATTACAATGGCCCCTCAAAAGCAGGGTATGT
TGACTTTGAAAATGGCCAGTGGGCCACAGATGACA
TCCCAGATGACTTGAATAGTATCCGCGCAGCACCA
GGTGAGTTTCGAGCCATCATGGAGATGCCCTCCTT
CTACAGTCATGGCTTGCCACGGTGTTCACCCTACA
AGAAACTGACAGAGTGCCAGCTGAAGAACCCCATC
AGCGGGCTGTTAGAATATGCCCAGTTCGCTAGTCA
AACCTGTGAGTTCAACATGATAGAGCAGAGTGGAC
CACCCCATGAACCTCGATTTAAATTCCAGGTTGTC
ATCAATGGCCGAGAGTTTCCCCCAGCTGAAGCTGG
AAGCAAGAAAGTGGCCAAGCAGGATGCAGCTATGA
AAGCCATGACAATTCTGCTAGAGGAAGCCAAAGCC
AAGGACAGTGGAAAATCAGAAGAATCATCCCACTA
TTCCACAGAGAAAGAATCAGAGAAGACTGCAGAGT
CCCAGACCCCCACCCCTTCAGCCACATCCTTCTTT
TCTGGGAAGAGCCCCGTCACCACACTGCTTGAGTG
TATGCACAAATTGGGGAACTCCTGCGAATTCCGTC
TCCTGTCCAAAGAAGGCCCTGCCCATGAACCCAAG
TTCCAATACTGTGTTGCAGTGGGAGCCCAAACTTT
CCCCAGTGTGAGTGCTCCCAGCAAGAAAGTGGCAA
AGCAGATGGCCGCAGAGGAAGCCATGAAGGCCCTG
CATGGGGAGGCGACCAACTCCATGGCTTCTGATAA
CCAGCCTGAAGGTATGATCTCAGAGTCACTTGATA
ACTTGGAATCCATGATGCCCAACAAGGTCAGGAAG
ATTGGCGAGCTCGTGAGATACCTGAACACCAACCC
TGTGGGTGGCCTTTTGGAGTACGCCCGCTCCCATG
GCTTTGCTGCTGAATTCAAGTTGGTCGACCAGTCC
GGACCTCCTCACGAGCCCAAGTTCGTTTACCAAGC
AAAAGTTGGGGGTCGCTGGTTCCCAGCCGTCTGCG
CACACAGCAAGAAGCAAGGCAAGCAGGAAGCAGCA
GATGCGGCTCTCCGTGTCTTGATTGGGGAGAACGA
GAAGGCAGAACGCATGGGTTTCACAGAGGTAACCC
CAGTGACAGGGGCCAGTCTCAGAAGAACTATGCTC
CTCCTCTCAAGGTCCCCAGAAGCACAGCCAAAGAC
ACTCCCTCTCACTGGCAGCACCTTCCATGACCAGA
TAGCCATGCTGAGCCACCGGTGCTTCAACACTCTG
ACTAACAGCTTCCAGCCCTCCTTGCTCGGCCGCAA
GATTCTGGCCGCCATCATTATGAAAAAAGACTCTG
AGGACATGGGTGTCGTCGTCAGCTTGGGAACAGGG
AATCGCTGTGTGAAAGGAGATTCTCTCAGCCTAAA
AGGAGAAACTGTCAATGACTGCCATGCAGAAATAA
TCTCCCGGAGAGGCTTCATCAGGTTTCTCTACAGT
GAGTTAATGAAATACAACTCCCAGACTGCGAAGGA
TAGTATATTTGAACCTGCTAAGGGAGGAGAAAAGC
TCCAAATAAAAAAGACTGTGTCATTCCATCTGTAT
ATCAGCACTGCTCCGTGTGGAGATGGCGCCCTCTT
TGACAAGTCCTGCAGCGACCGTGCTATGGAAAGCA
CAGAATCCCGCCACTACCCTGTCTTCGAGAATCCC
AAACAAGGAAAGCTCCGCACCAAGGTGGAGAACGG
AGAAGGCACAATCCCTGTGGAATCCAGTGACATTG
TGCCTACGTGGGATGGCATTCGGCTCGGGGAGAGA
CTCCGTACCATGTCCTGTAGTGACAAAATCCTACG
CTGGAACGTGCTGGGCCTGCAAGGGGCACTGTTGA
CCCACTTCCTGCAGCCCATTTATCTCAAATCTGTC
ACATTGGGTTACCTTTTCAGCCAAGGGCATCTGAC
CCGTGCTATTTGCTGTCGTGTGACAAGAGATGGGA
GTGCATTTGAGGATGGACTACGACATCCCTTTATT
GTCAACCACCCCAAGGTTGGCAGAGTCAGCATATA
TGATTCCAAAAGGCAATCCGGGAAGACTAAGGAGA
CAAGCGTCAACTGGTGTCTGGCTGATGGCTATGAC
CTGGAGATCCTGGACGGTACCAGAGGCACTGTGGA
TGGGCCACGGAATGAATTGTCCCGGGTCTCCAAAA
AGAACATTTTTCTTCTATTTAAGAAGCTCTGCTCC
TTCCGTTACCGCAGGGATCTACTGAGACTCTCCTA
TGGTGAGGCCAAGAAAGCTGCCCGTGACTACGAGA
CGGCCAAGAACTACTTCAAAAAAGGCCTGAAGGAT
ATGGGCTATGGGAACTGGATTAGCAAACCCCAGGA
GGAAAAGAACTTTTATCTCTGCCCAGTATAG
amino acid sequence:
(SEQ ID NO: 46)
MAEIKEKICDYLENVSDSSALNLAKNIGLTKARDI
NAVLIDMERQGDVYRQGTTPPIWHLTDKKRERMQI
KRNTNSVPETAPAAIPETKRNAEFLTCNIPTSNAS
NNMVTTEKVENGQEPVIKLENKQEARPEPARLKPP
VHYNGPSKAGYVDFENGQWATDDIPDDLNSIKAAP
GEFRAIMEMPSFYSHGLPRCSPYKKLTECQLKNPI
SGLLEYAQFASQTCEFNMIEQSGPPHEPRFKFQVV
INGREFPPAEAGSKKVAKQDAAMKAMTILLEEAKA
KDSGKSEESSHYSTEKESEKTAESQTPTPSATSFF
SGKSPVTTLLECMHKLGNSCEFRLLSKEGPAHEPK
FQYCVAVGAQTEPSVSAPSKKVAKQMAAEEAMKAL
HGEATNSMASDNQPEGMISESLDNLESMMPNKVRK
IGELVRYLNTNPVGGLLEYARSHGFAAEFKLVDQS
GPPHEPKEVYQAKVGGRWEPAVCAHSKKQGKQEAA
DAALRVLIGENEKAERMGFTEVTPVTGASLRRTML
LLSKSPEAQPKTLPLTGSTFHDQIAMLSHRCFNTL
TNSFQPSLLGRKILAAIIMKKDSEDMGVVVSLGTG
NRCVKGDSLSLKGETVNDCHAEIISRKGFIRFLYS
ELMKYNSQTAKDSIFEPAKGGEKLQIKKTVSFHLY
ISTAPCGDGALFDKSCSDRAMESTESRHYPVFENP
KQGKLRTKVENGEGTIPVESSDIVPTWDGIRLGER
LRTMSCSDKILKWNVLGLQGALLTHFLQPIYLKSV
TLGYLFSQGHLTRAICCRVTRDGSAFEDGLRHPFI
VNHPKVGRVSIYDSKRQSGKTKETSVNWCLADGYD
LEILDGTRGTVDGPKNELSRVSKKNIFLLFKKLCS
FRYRKDLLRLSYGEAKKAARDYETAKNYFKKGLKD
MGYGNWISKPQEEKNFYLCPV

According to a preferred embodiment, amino acid residue E1008 is mutated in hADAR1p150. Particularly preferred is the mutation E1008Q, a hyperactive mutant. Further preferred mutants include E1008Y, E1008F, E1008W, E1008H, E1008L, E1008M, E10081 and E1008V, which have reduced activity and are preferably used in connection with an artificial nucleic acid having an abasic site in the targeting sequence at the position corresponding to the nucleotide to be edited.

hADAR1p110:
nucleic acid sequence:
(SEQ ID NO: 47)
ATGGCCGAGATCAAGGAGAAAATCTGCGACTATCT
CTTCAATGTGTCTGACTCCTCTGCCCTGAATTTGG
CTAAAAATATTGGCCTTACCAAGGCCCGAGATATA
AATGCTGTGCTAATTGACATGGAAAGGCAGGGGGA
TGTCTATAGACAAGGGACAACCCCTCCCATATGGC
ATTTGACAGACAAGAAGCGAGAGAGGATGCAAATC
AAGAGAAATACGAACAGTGTTCCTGAAACCGCTCC
AGCTGCAATCCCTGAGACCAAAAGAAACGCAGAGT
TCCTCACCTGTAATATACCCACATCAAATGCCTCA
AATAACATGGTAACCACAGAAAAAGTGGAGAATGG
GCAGGAACCTGTCATAAAGTTAGAAAACAGGCAAG
AGGCCAGACCAGAACCAGCAAGACTGAAACCACCT
GTTCATTACAATGGCCCCTCAAAAGCAGGGTATGT
TGACTTTGAAAATGGCCAGTGGGCCACAGATGACA
TCCCAGATGACTTGAATAGTATCCGCGCAGCACCA
GGTGAGTTTCGAGCCATCATGGAGATGCCCTCCTT
CTACAGTCATGGCTTGCCACGGTGTTCACCCTACA
AGAAACTGACAGAGTGCCAGCTGAAGAACCCCATC
AGCGGGCTGTTAGAATATGCCCAGTTCGCTAGTCA
AACCTGTGAGTTCAACATGATAGAGCAGAGTGGAC
CACCCCATGAACCTCGATTTAAATTCCAGGTTGTC
ATCAATGGCCGAGAGTTTCCCCCAGCTGAAGCTGG
AAGCAAGAAAGTGGCCAAGCAGGATGCAGCTATGA
AAGCCATGACAATTCTGCTAGAGGAAGCCAAAGCC
AAGGACAGTGGAAAATCAGAAGAATCATCCCACTA
TTCCACAGAGAAAGAATCAGAGAAGACTGCAGAGT
CCCAGACCCCCACCCCTTCAGCCACATCCTTCTTT
TCTGGGAAGAGCCCCGTCACCACACTGCTTGAGTG
TATGCACAAATTGGGGAACTCCTGCGAATTCCGTC
TCCTGTCCAAAGAAGGCCCTGCCCATGAACCCAAG
TTCCAATACTGTGTTGCAGTGGGAGCCCAAACTTT
CCCCAGTGTGAGTGCTCCCAGCAAGAAAGTGGCAA
AGCAGATGGCCGCAGAGGAAGCCATGAAGGCCCTG
CATGGGGAGGCGACCAACTCCATGGCTTCTGATAA
CCAGCCTGAAGGTATGATCTCAGAGTCACTTGATA
ACTTGGAATCCATGATGCCCAACAAGGTCAGGAAG
ATTGGCGAGCTCGTGAGATACCTGAACACCAACCC
TGTGGGTGGCCTTTTGGAGTACGCCCGCTCCCATG
GCTTTGCTGCTGAATTCAAGTTGGTCGACCAGTCC
GGACCTCCTCACGAGCCCAAGTTCGTTTACCAAGC
AAAAGTTGGGGGTCGCTGGTTCCCAGCCGTCTGCG
CACACAGCAAGAAGCAAGGCAAGCAGGAAGCAGCA
GATGCGGCTCTCCGTGTCTTGATTGGGGAGAACGA
GAAGGCAGAACGCATGGGTTTCACAGAGGTAACCC
CAGTGACAGGGGCCAGTCTCAGAAGAACTATGCTC
CTCCTCTCAAGGTCCCCAGAAGCACAGCCAAAGAC
ACTCCCTCTCACTGGCAGCACCTTCCATGACCAGA
TAGCCATGCTGAGCCACCGGTGCTTCAACACTCTG
ACTAACAGCTTCCAGCCCTCCTTGCTCGGCCGCAA
GATTCTGGCCGCCATCATTATGAAAAAAGACTCTG
AGGACATGGGTGTCGTCGTCAGCTTGGGAACAGGG
AATCGCTGTGTGAAAGGAGATTCTCTCAGCCTAAA
AGGAGAAACTGTCAATGACTGCCATGCAGAAATAA
TCTCCCGGAGAGGCTTCATCAGGTTTCTCTACAGT
GAGTTAATGAAATACAACTCCCAGACTGCGAAGGA
TAGTATATTTGAACCTGCTAAGGGAGGAGAAAAGC
TCCAAATAAAAAAGACTGTGTCATTCCATCTGTAT
ATCAGCACTGCTCCGTGTGGAGATGGCGCCCTCTT
TGACAAGTCCTGCAGCGACCGTGCTATGGAAAGCA
CAGAATCCCGCCACTACCCTGTCTTCGAGAATCCC
AAACAAGGAAAGCTCCGCACCAAGGTGGAGAACGG
AGAAGGCACAATCCCTGTGGAATCCAGTGACATTG
TGCCTACGTGGGATGGCATTCGGCTCGGGGAGAGA
CTCCGTACCATGTCCTGTAGTGACAAAATCCTACG
CTGGAACGTGCTGGGCCTGCAAGGGGCACTGTTGA
CCCACTTCCTGCAGCCCATTTATCTCAAATCTGTC
ACATTGGGTTACCTTTTCAGCCAAGGGCATCTGAC
CCGTGCTATTTGCTGTCGTGTGACAAGAGATGGGA
GTGCATTTGAGGATGGACTACGACATCCCTTTATT
GTCAACCACCCCAAGGTTGGCAGAGTCAGCATATA
TGATTCCAAAAGGCAATCCGGGAAGACTAAGGAGA
CAAGCGTCAACTGGTGTCTGGCTGATGGCTATGAC
CTGGAGATCCTGGACGGTACCAGAGGCACTGTGGA
TGGGCCACGGAATGAATTGTCCCGGGTCTCCAAAA
AGAACATTTTTCTTCTATTTAAGAAGCTCTGCTCC
TTCCGTTACCGCAGGGATCTACTGAGACTCTCCTA
TGGTGAGGCCAAGAAAGCTGCCCGTGACTACGAGA
CGGCCAAGAACTACTTCAAAAAAGGCCTGAAGGAT
ATGGGCTATGGGAACTGGATTAGCAAACCCCAGGA
GGAAAAGAACTTTTATCTCTGCCCAGTATAG
amino acid sequence:
(SEQ ID NO: 48)
MAEIKEKICDYLFNVSDSSALNLAKNIGLTKARDI
NAVLIDMERQGDVYRQGTTPPIWHLTDKKRERMQI
KRNTNSVPETAPAAIPETKRNAEFLTCNIPTSNAS
NNMVTTEKVENGQEPVIKLENRQEARPEPARLKPP
VHYNGPSKAGYVDFENGQWATDDIPDDLNSIRAAP
GEFRAIMEMPSFYSHGLPRCSPYKKLTECQL
KNPISGLLEYAQFASQTCEFNMIEQSGPPHEPRFK
FQVVINGREFPPAEAGSKKVAKQDAAMKAMTILLE
EAKAKDSGKSEESSHYSTEKESEKTAESQTPTPSA
TSFFSGKSPVTTLLECMHKLGNSCEFRLLSKEGPA
HEPKFQYCVAVGAQTFPSVSAPSKKVAKQMAAEEA
MKALHGEATNSMASDNQPEGMISESLDNLESMMPN
KVRKIGELVRYLNINPVGGLLEYARSHGFAAEFKL
VDQSGPPHEPKFVYQAKVGGRWFPAVCAHSKKQGK
QEAADAALRVLIGENEKAERMGFTEVTPVTGASLR
RTMLLLSRSPEAQPKTLPLTGSTFHDQIAMLSHRC
FNTLTNSFQPSLLGRKILAAIIMKKDSEDMGVVVS
LGTGNRCVKGDSLSLKGETVNDCHAEIISRRGFIR
FLYSELMKYNSQTAKDSIFEPAKGGEKLQIKKTVS
FHLYISTAPCGDGALFDKSCSDRAMESTESRHYPV
FENPKQGKLRTKVENGEGTIPVESSDIVPTWDGIR
LGERLRTMSCSDKILRWNVLGLQGALLTHFLQPIY
LKSVTLGYLFSQGHLTRAICCRVTRDGSAFEDGLR
HPFIVNHPKVGRVSIYDSKRQSGKTKETSVNWCLA
DGYDLEILDGTRGTVDGPRNELSRVSKKNIFLLFK
KLCSFRYRRDLLRLSYGEAKKAARDYETAKNYFKK
GLKDMGYGNWISKPQEEKNFYLCPV

According to a preferred embodiment, amino acid residue E713 is mutated in hADAR1p110. Particularly preferred is the mutation E713Q, a hyperactive mutant. Further preferred mutants include E713Y, E713F, E713W, E713H, E713L, E713M, E713I and E713V, which have reduced activity and are preferably used in connection with an artificial nucleic acid having an abasic site in the targeting sequence at the position corresponding to the nucleotide to be edited.

hADAR2:
nucleic acid sequence:
(SEQ ID NO: 49)
ATGGATATAGAAGATGAAGAAAACATGAGTTCCAG
CAGCACTGATGTGAAGGAAAACCGCAATCTTGGAC
AACGTGTCCCCCAAGGATGGCAGCACACCTGTGGC
CTGGCGAGGGCTCTCAGCTCTCCAATTGGGGGTGG
TGGTGGCCCCGGCAGAAAGCGGCCCCTGGAGGAGG
GCAGCAATGGCCACTCCAATGTACCGCCTGAAGAA
AAGGAGGAAAACACCAGGGCCCGTCCTCCCCAAGA
ACGCCCTGATGCAGTCTGAATGAGATCAAGCCTGG
TTTTTTTGCAGTACACACTCCTGTCCCAGACTGGG
CCCGTGCACGCGCCTTTTGTTTGTCATGTCTGTGG
AGGTGAATGGCCAGGTTTTTTTTTGAGGGCTCTGG
TCCCACAAAGAAAAATGGCAAAACTCCATGCTGCT
GAGAAGGCCTTGAGGTCTTTTTTTCGTTCAGTTTC
CTAATGCCTCTGAGGCTCCACCTGGCCATGGGGAG
GACCCTGTCTGTCAACACGGACTTCACATCTGACC
AGGCCGACTTTTTTCCTCTGACACGCTCTTCAATG
GTTTTGAAACTCCTGACAAGGCGGAGCCTCCCTTT
TACGTGGGCTCCATATGGGGATGACTCCTTCAGTT
CCAGCGGGGACCTCAGCTTGTCTGCTTCCCCGGTG
CCTGCCAGCTCTAGCCCAGCCTCCTCTCCCTGCCT
TACCACCATTCCCACCCCCGAGTGGGAAGAATCCC
GTGATGTATCTTGAACGAACTGCGCCCAGGACTCA
AGTATGACTTCCTCTCCGAGAGCGGGGAGAGCCAT
GTCCAAGAGCTTCGTCATGTCTGTGGTCGTGGATG
GTCAGTTCTTTGAAGGCTCGGGGAGAAACAAGTAA
GCTTGCCAAGGCCCGGGCTGCGCAGTCTGCCCTGG
CCGCCATTTTTTTTTAACTTGCACTTGGATCATGA
CGCCATCTCGCCAGCCTATTCCCAGTGAGGGTCTT
CAGCTGCATTTTTTTACCGCAGGTTTTTTTTAGCT
GATCGCTGTCTCACGCCTGGTCCTGGGTAAGTTTG
GTGACCTGACCGACAACTTCTCCTCCCCTCACGCT
TCGCAGAAAAGTGCTGGCTGGAGTCGTCATGACAA
CAGGCACAGATGTTTTTAAAGATGCCAAGGTGTAT
AAGTGTTTCTACAGGAACAAAATGTATTAATGGTG
AATACATGAGTGATCGTGGCCTTGCATTATAATGA
CTGCCATGCAGAAATAATATCTCGGAGATCCTTGC
TCAGATTTCTTTATACACAACTTGAGCTTTTTTAC
TTAAATAACAAAGATGATCAAAAAAGATCCATCTT
TCAGAAATCAGAGCGAGGGGGGTTTTTTATGGCTG
AAGGAGAATGTCCAGTTTTTTCATCTGTACATCAG
CACCTCTCCCTGTGGAGATGCCAGAATCTTTCTCA
CCACATGAGCCAATCCTGGAAGAACCAGCAGATAG
ACACCCAAATCGTAAAGCAAGAGGATCAGCTACGG
ACCAAAATAGAGTCTGGTGAGGGGACGATTCCAGT
GCGCTCCAATGCGAGCATCCTAAACGTGGGACGGG
GTGCTGCAAGGGGAGCGGCTGCTCACCATGTCCTG
CAGTGACAAGATTGTCACGCTGGAACGTGGTGTGG
CATCCAGGGTTCCCTGCTCAGCATTTTCGTGGAGC
CCATTTACTTTTTCTTCGAGCATCATCCTGGGCAG
CCTTTTTTTACCACGGGGACCACCTTTCCAGGGCC
ATGTACCAGCGGATCTTCCAACATAGAGGACCTGC
CACCTCTCTACACCCTCAACAAGCCTTTGCTCAGT
GGCATCAGCAATTGCAGAAGCACGGCAGCCAGGGA
AGGCCCCCAACTTCAGTGTCAACTGGACGGTAGGC
GACTCCTGCTATTGAGGTCATCAACGCCACGACTG
GGAAGGATGAGCTGGGCCGCGCGTCCCGCCTGTGT
ATAGCACGCGTTGTACTGTCGCTGGATGCGTGTGC
ACGGCAAGGTTCCCTCCCACTTACTACGCTCCTAA
GATTTTTACCAAACCCAACGTGTACCATGAGTCCA
AGCTGGCGGCAAAGGAGTACCAGGCCGCCATAGGC
GCGTCTGTTCACAGCCTTCATCAAGGCGGGGCTGG
GGGCCTGGGTGGAGAAGCCCACCGTAGCAGGACCA
GTTTTTCTCACTCACG
amino acid sequence:
(SEQ ID NO: 50)
MDIEDEENMSSSSTDVKENRNLDNVSPKDGSTPGP
GEGSQLSNGGGGGPGRKRPLEEGSNGHSKYRLKKR
RKTPGPVLPKNALMQLNEIKPGLQYTLLSQTGPVH
APLEVMSVEVNGQVFEGSGPTKKKAKLHAAEKALR
SEVQFPNASEAHLAMGRTLSVNTDFTSDQADEPDT
LENGFETPDKAEPPFYVGSNGDDSFSSSGDLSLSA
SPVPASLAQPPLPALPPEPPPSGKNPVMILNELRP
GLKYDELSESGESHAKSFVMSVVVDGQFFEGSGRN
KKLAKARAAQSALAAIFNLHLDQTPSRQPIPSEGL
QLHLPQVLADAVSRLVLGKFGDLTDNFSSPHARRK
VLAGVVMTTGTDVKDAKVISVSTGTKCINGEYMSD
RGLALNDCHAEIISRRSLLRFLYTQLELYLNNKDD
QKRSIFQKSERGGFRLKENVQFHLYISTSPCGDAR
IFSPHEPILEEPADRHPNRKARGQLRTKIESGEGT
IPVRSNASIQTWDGVLQGERLLTMSCSDKIARWNV
VGIQGSLLSIFVEPIYESSIILGSLYHGDHLSRAM
YQRISNIEDLPPLYTLNKPLLSGISNAEARQPGKA
PNESVNWTVGDSAIEVINATTGKDELGRASRLCKH
ALYCRWMRVHGKVPSHLLRSKITKPNVYHESKLAA
KEYQAAKARLFTAFIKAGLGAWVEKPTEQDQFSLT

According to a preferred embodiment, amino acid residue E488 is mutated in hADAR2. Particularly preferred is the mutation E488Q, a hyperactive mutant. Further preferred mutants include E488Y, E488F, E488W, E488H, E488L, E488M, E488I and E488V, which have reduced activity and are preferably used in connection with an artificial nucleic acid having an abasic site in the targeting sequence at the position corresponding to the nucleotide to be edited. Further preferred sites, which may be mutated in hADAR2 comprise 1456 or T490, and further also R348, R470, H471, R474, S495, R510, K594, R477 or R481.

SNAPf-ADAR1:
nucleic acid sequence:
(SEQ ID NO: 51)
ATGGACAAAGACTGCGAAATGAAGCGCACCACCCT
GGATAGCCCTCTGGGCAAGCTGGAACTGTCTGGGT
GCGAACAGGGCCTGCACCGTATCATCTTCCTGGGC
AAAGGAACATCTGCCGCCGACGCCGTGGAAGTGCC
TGCCCCAGCCGCCGTGCTGGGCGGACCAGAGCCAC
TGATGCAGGCCACCGCCTGGCTCAACGCCTACTTT
CACCAGCCTGAGGCCATCGAGGAGTTCCCTGTGCC
AGCCCTGCACCACCCAGTGTTCCAGCAGGAGAGCT
TTACCCGCCAGGTGCTGTGGAAACTGCTGAAAGTG
GTGAAGTTCGGAGAGGTCATCAGCTACAGCCACCT
GGCCGCCCTGGCCGGCAATCCCGCCGCCACCGCCG
CCGTGAAAACCGCCCTGAGCGGAAATCCCGTGCCC
ATTCTGATCCCCTGCCACCGGGTGGTGCAGGGCGA
CCTGGACGTGGGGGGCTACGAGGGCGGGCTCGCCG
TGAAAGAGTGGCTGCTGGCCCACGAGGGCCACAGA
CTGGGCAAGCCTGGGCTGGGTCCTGCAGGCGGAGG
CGCGCCAGGGTCTGGCGGCGGCAGTAAGGCAGAAC
GCATGGGTTTCACAGAGGTAACCCCAGTGACAGGG
GCCAGTCTCAGAAGAACTATGCTCCTCCTCTCAAG
GTCCCCAGAAGCACAGCCAAAGACACTCCCTCTCA
CTGGCAGCACCTTCCATGACCAGATAGCCATGCTG
AGCCACCGGTGCTTCAACACTCTGACTAACAGCTT
CCAGCCCTCCTTGCTCGGCCGCAAGATTCTGGCCG
CCATCATTATGAAAAAAGACTCTGAGGACATGGGT
GTCGTCGTCAGCTTGGGAACAGGGAATCGCTGTGT
AAAAGGAGATTCTCTCAGCCTAAAAGGAGAAACTG
TCAATGACTGCCATGCAGAAATAATCTCCCGGAGA
GGCTTCATCAGGTTTCTCTACAGTGAGTTAATGAA
ATACAACTCCCAGACTGCGAAGGATAGTATATTTG
AACCTGCTAAGGGAGGAGAAAAGCTCCAAATAAAA
AAGACTGTGTCATTCCATCTGTATATCAGCACTGC
TCCGTGTGGAGATGGCGCCCTCTTTGACAAGTCCT
GCAGCGACCGTGCTATGGAAAGCACAGAATCCCGC
CACTACCCTGTCTTCGAGAATCCCAAACAAGGAAA
GCTCCGCACCAAGGTGGAGAACGGAGAAGGCACAA
TCCCTGTGGAATCCAGTGACATTGTGCCTACGTGG
GATGGCATTCGGCTCGGGGAGAGACTCCGTACCAT
GTCCTGTAGTGACAAAATCCTACGCTGGAACGTGC
TGGGCCTGCAAGGGGCACTGTTGACCCACTTCCTG
CAGCCCATTTATCTCAAATCTGTCACATTGGGTTA
CCTTTTCAGCCAAGGGCATCTGACCCGTGCTATTT
GCTGTCGTGTGACAAGAGATGGGAGTGCATTTGAG
GATGGACTACGACATCCCTTTATTGTCAACCACCC
CAAGGTTGGCAGAGTCAGCATATATGATTCCAAAA
GGCAATCCGGGAAGACTAAGGAGACAAGCGTCAAC
TGGTGTCTGGCTGATGGCTATGACCTGGAGATCCT
GGACGGTACCAGAGGCACTGTGGATGGGCCACGGA
ATGAATTGTCCCGGGTCTCCAAAAAGAACATTTTT
CTTCTATTTAAGAAGCTCTGCTCCTTCCGTTACCG
CAGGGATCTACTGAGACTCTCCTATGGTGAGGCCA
AGAAAGCTGCCCGTGACTACGAGACGGCCAAGAAC
TACTTCAAAAAAGGCCTGAAGGATATGGGCTATGG
GAACTGGATTAGCAAACCCCAGGAGGAAAAGAACT
TTTATCTCTGCCCAGTA
amino acid sequence:
(SEQ ID NO: 52)
MDKDCEMKRTTLDSPLGKLELSGCEQGLHRIIFLG
KGTSAADAVEVPAPAAVLGGPEPLMQATAWLNAYF
HQPEAIEEFPVPALHHPVFQQESFTRQVLWKLLKV
VKFGEVISYSHLAALAGNPAATAAVKTALSGNPVP
ILIPCHRVVQGDLDVGGYEGGLAVKEWLLAHEGHR
LGKPGLGPAGGGAPGSGGGSKAERMGFTEVTPVTG
ASLRRTMLLLSRSPEAQPKTLPLTGSTFHDQIAML
SHRCFNTLTNSFQPSLLGRKILAAIIMKKDSEDMG
VVVSLGTGNRCVKGDSLSLKGETVNDCHAEIISRR
GFIRFLYSELMKYNSQTAKDSIFEPAKGGEKLQIK
KTVSFHLYISTAPCGDGALFDKSCSDRAMESTESR
HYPVFENPKQGKLRTKVENGEGTIPVESSDIVPTW
DGIRLGERLRTMSCSDKILRWNVLGLQGALLTHFL
QPIYLKSVTLGYLFSQGHLTRAICCRVTRDGSAFE
DGLRHPFIVNHPKVGRVSIYDSKRQSGKTKETSVN
WCLADGYDLEILDGTRGTVDGPRNELSRVSKKNIF
LLFKKLCSFRYRRDLLRLSYGEAKKAARDYETAKN
YFKKGLKDMGYGNWISKPQEEKNFYLCPV

According to a preferred embodiment, amino acid residue E406 is mutated in SNAPf-ADAR1. Particularly preferred is the mutation E406Q, a hyperactive mutant. Further preferred mutants include E406Y, E406F, E406W, E406H, E406L, E406M, E406I and E406V, which have reduced activity and are preferably used in connection with an artificial nucleic acid having an abasic site in the targeting sequence at the position corresponding to the nucleotide to be edited.

SNAPf-ADAR2:
nucleic acid sequence:
(SEQ ID NO: 53)
ATGGACAAAGACTGCGAAATGAAGCGCACCACCCT
GGATAGCCCTCTGGGCAAGCTGGAACTGTCTGGGT
GCGAACAGGGCCTGCACCGTATCATCTTCCTGGGC
AAAGGAACATCTGCCGCCGACGCCGTGGAAGTGCC
TGCCCCAGCCGCCGTGCTGGGCGGACCAGAGCCAC
TGATGCAGGCCACCGCCTGGCTCAACGCCTACTTT
CACCAGCCTGAGGCCATCGAGGAGTTCCCTGTGCC
AGCCCTGCACCACCCAGTGTTCCAGCAGGAGAGCT
TTACCCGCCAGGTGCTGTGGAAACTGCTGAAAGTG
GTGAAGTTCGGAGAGGTCATCAGCTACAGCCACCT
GGCCGCCCTGGCCGGCAATCCCGCCGCCACCGCCG
CCGTGAAAACCGCCCTGAGCGGAAATCCCGTGCCC
ATTCTGATCCCCTGCCACCGGGTGGTGCAGGGCGA
CCTGGACGTGGGGGGCTACGAGGGCGGGCTCGCCG
TGAAAGAGTGGCTGCTGGCCCACGAGGGCCACAGA
CTGGGCAAGCCTGGGCTGGGTCCTGCAGGCGGAGG
CGCGCCAGGGTCTGGCGGCGGCAGTAAGAAGCTTG
CCAAGGCCCGGGCTGCGCAGTCTGCCCTGGCCGCCA
TTTTTAACTTGCACTTGGATCAGACGCCATCTCGCCA
GCCTATTCCCAGTGAGGGTCTTCAGCTGCATTTACC
GCAGGTTTTAGCTGACGCTGTCTCACGCCTGGTCCT
GGGTAAGTTTGGTGACCTGACCGACAACTTCTCCT
CCCCTCACGCTCGCAGAAAAGTGCTGGCTGGAGTC
GTCATGACAACAGGCACAGATGTTAAAGATGCCAA
GGTGATAAGTGTTTCTACAGGAACAAAATGTATTA
ATGGTGAATACATGAGTGATCGTGGCCTTGCATTA
AATGACTGCCATGCAGAAATAATATCTCGGAGATC
CTTGCTCAGATTTCTTTATACACAACTTGAGCTTT
ACTTAAATAACAAAGATGATCAAAAAAGATCCATC
TTTCAGAAATCAGAGCGAGGGGGGTTTAGGCTGAA
GGAGAATGTCCAGTTTCATCTGTACATCAGCACCT
CTCCCTGTGGAGATGCCAGAATCTTCTCACCACAT
GAGCCAATCCTGGAAGAACCAGCAGATAGACACCC
AAATCGTAAAGCAAGAGGACAGCTACGGACCAAAA
TAGAGTCTGGTGAGGGGACGATTCCAGTGCGCTCC
AATGCGAGCATCCAAACGTGGGACGGGGTGCTGCA
AGGGGAGCGGCTGCTCACCATGTCCTGCAGTGACA
AGATTGCACGCTGGAACGTGGTGGGCATCCAGGGA
TCCCTGCTCAGCATTTTCGTGGAGCCCATTTACTT
CTCGAGCATCATCCTGGGCAGCCTTTACCACGGGG
ACCACCTTTCCAGGGCCATGTACCAGCGGATCTCC
AACATAGAGGACCTGCCACCTCTCTACACCCTCAA
CAAGCCTTTGCTCAGTGGCATCAGCAATGCAGAAG
CACGGCAGCCAGGGAAGGCCCCCAACTTCAGTGTC
AACTGGACGGTAGGCGACTCCGCTATTGAGGTCAT
CAACGCCACGACTGGGAAGGATGAGCTGGGCCGCG
CGTCCCGCCTGTGTAAGCACGCGTTGTACTGTCGC
TGGATGCGTGTGCACGGCAAGGTTCCCTCCCACTT
ACTACGCTCCAAGATTACCAAGCCCAACGTGTACC
ATGAGTCCAAGCTGGCGGCAAAGGAGTACCAGGCC
GCCAAGGCGCGTCTGTTCACAGCCTTCATCAAGGC
GGGGCTGGGGGCCTGGGTGGAGAAGCCCACCGAGC
AGGACCAGTTCTCACTCACGCCC
amino acid sequence:
(SEQ ID NO: 54)
MDKDCEMKRTTLDSPLGKLELSGCEQGLHRIIFLG
KGTSAADAVEVPAPAAVLGGPEPLMQATAWLNAYF
HQPEAIEEFPVPALHHPVFQQESFTRQVLWKLLKV
VKFGEVISYSHLAALAGNPAATAAVKTALSGNPVP
ILIPCHRVVQGDLDVGGYEGGLAVKEWLLAHEGHR
LGKPGLGPAGGGAPGSGGGSKKLAKARAAQSALAA
IFNLHLDQTPSRQPIPSEGLQLHLPQVLADAVSRL
VLGKFGDLTDNFSSPHARRKVLAGVVMTTGTDVKD
AKVISVSTGTKCINGEYMSDRGLALNDCHAEII
SRRSLLRFLYTQLELYLNNKDDQKRSIFQKSERG
GFRLKENVQFHLYISTSPCGDARIFSPHEPILEEP
ADRHPNRKARGQLRTKIESGEGTIPVRSNASIQTW
DGVLQGERLLTMSCSDKIARWNVVGIQGSLLSIFV
EPIYESSIILGSLYHGDHLSRAMYQRISNIEDLPP
LYTLNKPLLSGISNAEARQPGKAPNFSVNWTVGDS
AIEVINAIIGKDELGRASRLCKHALYCRWMRVHGK
VPSHLLRSKITKPNVYHESKLAAKEYQAAKARLFT
AFIKAGLGAWVEKPTEQDQFSLTP

According to a preferred embodiment, amino acid residue E403 is mutated in hADAR2. Particularly preferred is the mutation E403Q, a hyperactive mutant. Further preferred mutants include E403Y, E403F, E403W, E403H, E403L, E403M, E403I and E403V, which have reduced activity and are preferably used in connection with an artificial nucleic acid having an abasic site in the targeting sequence at the position corresponding to the nucleotide to be edited. Further preferred sites, which may be mutated in hADAR2 comprise I371 or T405, and further also R263, R385, H386, R389, S410, R425, K509, R392 or R484.

mAPOBEC1-SNAP (mA1-SNAP),
C-to-U deaminase:
nucleic acid sequence:
(SEQ ID NO: 55)
ATGAGTTCCGAGACAGGCCCTGTAGCTGTTGATCC
CACTCTGAGGAGAAGAATTGAGCCCCACGAGTTTG
AAGTCTTCTTTGACCCCCGGGAGCTTCGGAAAGAG
ACCTGTCTGCTGTATGAGATCAACTGGGGTGGAAG
GCACAGTGTCTGGCGACACACGAGCCAAAACACCA
GCAACCACGTTGAAGTCAACTTCTTAGAAAAATTT
ACTACAGAAAGATACTTTCGTCCGAACACCAGATG
CTCCATTACCTGGTTCCTGTCCTGGAGTCCCTGCG
GGGAGTGCTCCAGGGCCATTACAGAGTTTCTGAGC
CGACACCCCTATGTAACTCTGTTTATTTACATAGC
ACGGCTTTATCACCACACGGATCAGCGAAACCGCC
AAGGACTCAGGGACCTTATTAGCAGCGGTGTGACT
ATCCAGATCATGACAGAGCAAGAGTATTGTTACTG
CTGGAGGAATTTCGTCAACTACCCCCCTTCAAACG
AAGCATATTGGCCAAGGTACCCCCATCTGTGGGTG
AAACTGTATGTACTGGAGCTCTACTGCATCATTTT
AGGACTTCCACCCTGTTTAAAAATTTTAAGAAG
AAAGCAACCTCAACTCACGTTTTTCACAATTACTC
TTCAAACCTGCCATTACCAAAGGATACCACCCCAT
CTCCTTTGGGCTACAGGGTTGAAAGGAGCGGCCGC
GACTGGCGCGCCAGGGGGATCCATGGACAAAGACT
GCGAAATGAAGCGCACCACCCTGGATAGCCCTCTG
GGCAAGCTGGAACTGTCTGGGTGCGAACAGGGCCT
GCACCGTATCATCTTCCTGGGCAAAGGAACATCTG
CCGCCGACGCCGTGGAAGTGCCTGCCCCAGCCGCC
GTGCTGGGCGGACCAGAGCCACTGATGCAGGCCAC
CGCCTGGCTCAACGCCTACTTTCACCAGCCTGAGG
CCATCGAGGAGTTCCCTGTGCCAGCCCTGCACCAC
CCAGTGTTCCAGCAGGAGAGCTTTACCCGCCAGGT
GCTGTGGAAACTGCTGAAAGTGGTGAAGTTCGGAG
AGGTCATCAGCTACAGCCACCTGGCCGCCCTGGCC
GGCAATCCCGCCGCCACCGCCGCCGTGAAAACCGC
CCTGAGCGGAAATCCCGTGCCCATTCTGATCCCCT
GCCACCGGGTGGTGCAGGGCGACCTGGACGTGGGG
GGCTACGAGGGCGGGCTCGCCGTGAAAGAGTGGCT
GCTGGCCCACGAGGGCCACAGACTGGGCAAGCCTG
GGCTGGGT
amino acid sequence:
(SEQ ID NO: 56)
MSSETGPVAVDPTLRRRIEPHEFEVFFDPRELRKE
TCLLYEINWGGRHSVWRHTSQNTSNHVEVNFLEKF
TTERYFRPNTRCSITVVELSWSPCGECSRAITERS
RHPYVTLFIYIARLYHHTDQRNRQGLRDLISSGVT
IQIMTEQEYCYCWRNFVNYPPSNEAYWPRYPHLWV
KLYVLELYCIILGLPPCLKILRRKQPQLTFFTITL
QTCHYQRIPPHLLWATGLKGAAATGAPGGSMDKDC
EMKRTTLDSPLGKLELSGCEQGLHRIIFLGKGTSA
ADAVEVPAPAAVLGGPEPLMQATAWLNAYFHQPEA
IEEFPVPALHHPVFQQESFTRQVLWKLLKVVKFGE
VISYSHLAALAGNPAATAAVKTALSGNPVPILIPC
HRVVQGDLDVGGYEGGLAVKEWLLAHEGHRLGKPG
LG
Halo-ADAR1Q:
nucleic acid sequence:
(SEQ ID NO: 57)
ATGGCAGAAATCGGTACTGGCTTTCCATTCGACCC
CCATTATGTGGAAGTCCTGGGCGAGCGCATGCACT
ACGTCGATGTTGGTCCGCGCGATGGCACCCCTGTG
CTGTTCCTGCACGGTAACCCGACCTCCTCCTACGT
GTGGCGCAACATCATCCCGCATGTTGCACCGACCC
ATCGCTGCATTGCTCCAGACCTGATCGGTATGGGC
AAATCCGACAAACCAGACCTGGGTTATTTCTTCGA
CGACCACGTCCGCTTCATGGATGCCTTCATCGAAG
CCCTGGGTCTGGAAGAGGTCGTCCTGGTCATTCAC
GACTGGGGCTCCGCTCTGGGTTTCCACTGGGCCAA
GCGCAATCCAGAGCGCGTCAAAGGTATTGCATTTA
TGGAGTTCATCCGCCCTATCCCGACCTGGGACGAA
TGGCCAGAATTTGCCCGCGAGACCTTCCAGGCCTT
CCGCACCACCGACGTCGGCCGCAAGCTGATCATCG
ATCAGAACGTTTTTATCGAGGGTACGCTGCCGATG
GGTGTCGTCCGCCCGCTGACTGAAGTCGAGATGGA
CCATTACCGCGAGCCGTTCCTGAATCCTGTTGACC
GCGAGCCACTGTGGCGCTTCCCAAACGAGCTGCCA
ATCGCCGGTGAGCCAGCGAACATCGTCGCGCTGGT
CGAAGAATACATGGACTGGCTGCACCAGTCCCCTG
TCCCGAAGCTGCTGTTCTGGGGCACCCCAGGCGTT
CTGATCCCACCGGCCGAAGCCGCTCGCCTGGCCAA
AAGCCTGCCTAACTGCAAGGCTGTGGACATCGGCC
CGGGTCTGAATCTGCTGCAAGAAGACAACCCGGAC
CTGATCGGCAGCGAGATCGCGCGCTGGCTGTCGAC
GCTCGAGAAGCCAACCCCTGCAGGCGGAGGCGCGC
CAGGGTCTGGCGGCGGCAGTAAGGCAGAACGCATG
GGTTTCACAGAGGTAACCCCAGTGACAGGGGCCAG
TCTCAGAAGAACTATGCTCCTCCTCTCAAGGTCCC
CAGAAGCACAGCCAAAGACACTCCCTCTCACTGGC
AGCACCTTCCATGACCAGATAGCCATGCTGAGCCA
CCGGTGCTTCAACACTCTGACTAACAGCTTCCAGC
CCTCCTTGCTCGGCCGCAAGATTCTGGCCGCCATC
ATTATGAAAAAAGACTCTGAGGACATGGGTGTCGT
CGTCAGCTTGGGAACAGGGAATCGCTGTGTAAAAG
GAGATTCTCTCAGCCTAAAAGGAGAAACTGTCAAT
GACTGCCATGCAGAAATAATCTCCCGGAGAGGCTT
CATCAGGTTTCTCTACAGTGAGTTAATGAAATACA
ACTCCCAGACTGCGAAGGATAGTATATTTGAACCT
GCTAAGGGAGGAGAAAAGCTCCAAATAAAAAAGAC
TGTGTCATTCCATCTGTATATCAGCACTGCTCCGT
GTGGAGATGGCGCCCTCTTTGACAAGTCCTGCAGC
GACCGTGCTATGGAAAGCACAGAATCCCGCCACTA
CCCTGTCTTCGAGAATCCCAAACAAGGAAAGCTCC
GCACCAAGGTGGAGAACGGACAAGGCACAATCCCT
GTGGAATCCAGTGACATTGTGCCTACGTGGGATGG
CATTCGGCTCGGGGAGAGACTCCGTACCATGTCCT
GTAGTGACAAAATCCTACGCTGGAACGTGCTGGGC
CTGCAAGGGGCACTGTTGACCCACTTCCTGCAGCC
CATTTATCTCAAATCTGTCACATTGGGTTACCTTT
TCAGCCAAGGGCATCTGACCCGTGCTATTTGCTGT
CGTGTGACAAGAGATGGGAGTGCATTTGAGGATGG
ACTACGACATCCCTTTATTGTCAACCACCCCAAGG
TTGGCAGAGTCAGCATATATGATTCCAAAAGGCAA
TCCGGGAAGACTAAGGAGACAAGCGTCAACTGGTG
TCTGGCTGATGGCTATGACCTGGAGATCCTGGACG
GTACCAGAGGCACTGTGGATGGGCCACGGAATGAA
TTGTCCCGGGTCTCCAAAAAGAACATTTTTCTTCT
ATTTAAGAAGCTCTGCTCCTTCCGTTACCGCAGGG
ATCTACTGAGACTCTCCTATGGTGAGGCCAAGAAA
GCTGCCCGTGACTACGAGACGGCCAAGAACTACTT
CAAAAAAGGCCTGAAGGATATGGGCTATGGGAACT
GGATTAGCAAACCCCAGGAGGAAAAGAACTTTTAT
CTCTGCCCAGTA
amino acid sequence:
(SEQ ID NO: 58)
MAEIGTGFPFDPHYVEVLGERMHYVDVGPRDGTPV
LFLHGNPTSSYVWRNIIPHVAPTHRCIAPDLIGMG
KSDKPDLGYFFDDHVRFMDAFIEALGLEEVVLVIH
DWGSALGFHWAKRNPERVKGIAFMEFIRPIPTWDE
WPEFARETFQAFRTTDVGRKLIIDQNVFIEGTLPM
GVVRPLTEVEMDHYREPFLNPVDREPLWRFPNELP
IAGEPANIVALVEEYMDWLHQSPVPKLLFWGTPGV
LIPPAEAARLAKSLPNCKAVDIGPGLNLLQEDNPD
LIGSEIARWLSTLEKPTPAGGGAPGSGGGSKAERM
GFTEVTPVTGASLRRTMLLLSRSPEAQPKTLPLTG
STFHDQIAMLSHRCFNTLTNSFQPSLLGRKILAAI
IMKKDSEDMGVVVSLGTGNRCVKGDSLSLKGETVN
DCHAEIISRRGFIRFLYSELMKYNSQTAKDSIFEP
AKGGEKLQIKKTVSFHLYISTAPCGDGALFDKSCS
DRAMESTESRHYPVFENPKQGKLRTKVENGQGTIP
VESSDIVPTWDGIRLGERLRTMSCSDKILRWNVLG
LQGALLTHFLQPIYLKSVTLGYLFSQGHLTRAICC
RVTRDGSAFEDGLRHPFIVNHPKVGRVSIYDSKRQ
SGKTKETSVNWCLADGYDLEILDGTRGTVDGPRNE
LSRVSKKNIFLLFKKLCSFRYRRDLLRLSYGEAKK
AARDYETAKNYEKKGLKDMGYGNWISKPQEEKNFY
LCPV

According to a preferred embodiment, the wild type amino acid residue E521 is mutated to Q, resulting in a hyperactive deaminase mutant. Further preferred mutants include E521Y, E521F, E521W, E521H, E521L, E521M and E521V, which have reduced activity and which are preferably used in connection with an artificial nucleic acid having an abasic site in the targeting sequence at the position corresponding to the nucleotide to be edited.

Clipf-ADAR1Q:
nucleic acid sequence:
(SEQ ID NO: 59)
ATGGACAAAGACTGCGAAATGAAGCGCACCACCCT
GGATAGCCCTCTGGGCAAGCTGGAACTGTCTGGGT
GCGAACAGGGCCTGCACCGTATCATCTTCCTGGGC
AAAGGAACATCTGCCGCCGACGCCGTGGAAGTGCC
TGCCCCAGCCGCCGTGCTGGGCGGACCAGAGCCAC
TGATCCAGGCCACCGCCTGGCTCAACGCCTACTTT
CACCAGCCTGAGGCCATCGAGGAGTTCCCTGTGCC
AGCCCTGCACCACCCAGTGTTCCAGCAGGAGAGCT
TTACCCGCCAGGTGCTGTGGAAACTGCTGAAAGTG
GTGAAGTTCGGAGAGGTCATCAGCGAGAGCCACCT
GGCCGCCCTGGTGGGCAATCCCGCCGCCACCGCCG
CCGTGAACACCGCCCTGGACGGAAATCCCGTGCCC
ATTCTGATCCCCTGCCACCGGGTGGTGCAGGGCGA
CAGCGACGTGGGGCCCTACCTGGGCGGGCTCGCCG
TGAAAGAGTGGCTGCTGGCCCACGAGGGCCACAGA
CTGGGCAAGCCTGGGCTGGGTCCTGCAGGCGGAGG
CGCGCCAGGGTCTGGCGGCGGCAGTAAGGCAGAAC
GCATGGGTTTCACAGAGGTAACCCCAGTGACAGGG
GCCAGTCTCAGAAGAACTATGCTCCTCCTCTCAAG
GTCCCCAGAAGCACAGCCAAAGACACTCCCTCTCA
CTGGCAGCACCTTCCATGACCAGATAGCCATGCTG
AGCCACCGGTGCTTCAACACTCTGACTAACAGCTT
CCAGCCCTCCTTGCTCGGCCGCAAGATTCTGGCCG
CCATCATTATGAAAAAAGACTCTGAGGACATGGGT
GTCGTCGTCAGCTTGGGAACAGGGAATCGCTGTGT
AAAAGGAGATTCTCTCAGCCTAAAAGGAGAAACTG
TCAATGACTGCCATGCAGAAATAATCTCCCGGAGA
GGCTTCATCAGGTTTCTCTACAGTGAGTTAATGAA
ATACAACTCCCAGACTGCGAAGGATAGTATATTTG
AACCTGCTAAGGGAGGAGAAAAGCTCCAAATAAAA
AAGACTGTGTCATTCCATCTGTATATCAGCACTGC
TCCGTGTGGAGATGGCGCCCTCTTTGACAAGTCCT
GCAGCGACCGTGCTATGGAAAGCACAGAATCCCGC
CACTACCCTGTCTTCGAGAATCCCAAACAAGGAAA
GCTCCGCACCAAGGTGGAGAACGGACAAGGCACAA
TCCCTGTGGAATCCAGTGACATTGTGCCTACGTGG
GATGGCATTCGGCTCGGGGAGAGACTCCGTACCAT
GTCCTGTAGTGACAAAATCCTACGCTGGAACGTGC
TGGGCCTGCAAGGGGCACTGTTGACCCACTTCCTG
CAGCCCATTTATCTCAAATCTGTCACATTGGGTTA
CCTTTTCAGCCAAGGGCATCTGACCCGTGCTATTT
GCTGTCGTGTGACAAGAGATGGGAGTGCATTTGAG
GATGGACTACGACATCCCTTTATTGTCAACC
ACCCCAAGGTTGGCAGAGTCAGCAT
ATATGATTCCAAAAGGCAATCCGGGAAGACTAAGG
AGACAAGCGTCAACTGGTGTCTGGCTGATGGCTAT
GACCTGGAGATCCTGGACGGTACCAGAGGCACTGT
GGATGGGCCACGGAATGAATTGTCCCGGGTCTCCA
AAAAGAACATTTTTCTTCTATTTAAGAAGCTCTGC
TCCTTCCGTTACCGCAGGGATCTACTGAGACTCTC
CTATGGTGAGGCCAAGAAAGCTGCCCGTGACTACG
AGACGGCCAAGAACTACTTCAAAAAAGGCCTGAAG
GATATGGGCTATGGGAACTGGATTAGCAAACCCCA
GGAGGAAAAGAACTTTTATCTCTGCCCAGTA
amino acid sequence:
(SEQ ID NO: 60)
MDKDCEMKRTTLDSPLGKLELSGCEQGLHRIIFLG
KGTSAADAVEVPAPAAVLGGPEPLIQATAWLNAYF
HQPEAIEEFPVPALHHPVFQQESFTRQVLWKLLKV
VKFGEVISESHLAALVGNPAATAAVNTALDGNPVP
ILIPCHRVVQGDSDVGPYLGGLAVKEWLLAHEGHR
LGKPGLGPAGGGAPGSGGGSKAERMGFTEVTPVTG
ASLRRTMLLLSRSPEAQPKTLPLTGSTFHDQIAML
SHRCFNTLTNSFQPSLLGRKILAAIIMKKDSEDMG
VVVSLGTGNRCVKGDSLSLKGETVNDCHAEIISRR
GFIRFLYSELMKYNSQTAKDSIFEPAKGGEKLQIK
KTVSFHLYISTAPCGDGALFDKSCSDRAMESTESR
HYPVFENPKQGKLRTKVENGQGTIPVESSDIVPTW
DGIRLGERLRTMSCSDKILRWNVLGLQGALLTHFL
QPIYLKSVTLGYLFSQGHLTRAICCRVTRDGSAFE
DGLRHPFIVNHPKVGRVSIYDSKRQSGKTKETSVN
WCLADGYDLEILDGTRGTVDGPRNELSRVSKKNIF
LLFKKLCSFRYRRDLLRLSYGEAKKAARDYETAKN
YFKKGLKDMGYGNWISKPQEEKNFYLCPV

According to a preferred embodiment, the wild type amino acid residue E406 is mutated to Q in Clipf-ADAR1, resulting in a hyperactive deaminase mutant. Further preferred mutants include E406Y, E406F, E406W, E406H, E406L, E406M and E406V, which have reduced activity and which are preferably used in connection with an artificial nucleic acid having an abasic site in the targeting sequence at the position corresponding to the nucleotide to be edited.

According to a preferred embodiment, the artificial nucleic acid described herein, which comprises a recruiting moiety with a nucleic acid recruiting motif (see respective section herein) is preferably used for site-directed editing of an RNA in the presence of an endogenous deaminase, preferably selected from the group consisting of hADAR1p110, hADAR1p150, hADAR2 and Apobec1, preferably as defined by the sequences as defined above, or a fragment or variant of any of these deaminases.

According to an alternative embodiment, the artificial nucleic acid described herein, which comprises a recruiting moiety with a coupling agent (see respective section herein) is preferably used for site-directed editing of an RNA in the presence of a tagged deaminase, preferably selected from the group consisting of SNAPf-ADAR1, SNAPf-ADAR2, mAPOBEC-SNAP, Halo-ADAR and Clipf-ADAR, preferably as defined by the sequences as defined above, or a fragment or variant of any of these deaminases.

Vector Comprising the Artificial Nucleic Acid

In one aspect, the present invention provides a vector comprising the artificial nucleic acid described herein.

The term ‘vector’ as used herein typically refers to a nucleic acid molecule, preferably to an artificial nucleic acid molecule. A vector in the context of the present invention is suitable for incorporating or harbouring a desired nucleic acid sequence, such as the nucleic acid sequence of the artificial nucleic acid or a fragment thereof. Such vectors may be storage vectors, expression vectors, cloning vectors, transfer vectors etc. A cloning vector may be, e.g., a plasmid vector or a bacteriophage vector. A transfer vector may be a vector, which is suitable for transferring nucleic acid molecules into cells or organisms, for example, viral vectors. Preferably, a vector in the sense of the present application comprises a cloning site, a selection marker, such as an antibiotic resistance factor, and a sequence suitable for multiplication of the vector, such as an origin of replication.

The vector may be an RNA vector or a DNA vector. Preferably, the vector is a DNA vector. The vector may be any vector known to the skilled person, such as a viral vector or a plasmid vector. Preferably, the vector is a plasmid vector, preferably a DNA plasmid vector. In certain embodiments, the vector is a viral vector, which is preferably selected from the group consisting of lentiviral vectors, retroviral vectors, adenoviral vectors, adeno-associated viral (AAV) vectors and hybrid vectors.

Preferably, the vector according to the present invention is suitable for producing the artificial nucleic acid molecule, preferably an RNA, according to the present invention. Thus, preferably, the vector comprises elements needed for transcription, such as a promoter, e.g. an RNA polymerase promoter. Preferably, the vector is suitable for transcription using eukaryotic, prokaryotic, viral or phage transcription systems, such as eukaryotic cells, prokaryotic cells, or eukaryotic, prokaryotic, viral or phage in vitro transcription systems. Thus, for example, the vector may comprise a promoter sequence, which is recognized by a polymerase, such as by an RNA polymerase, e.g. by a eukaryotic, prokaryotic, viral, or phage RNA polymerase. In a preferred embodiment, the vector comprises a phage RNA polymerase promoter such as an SP6, T3 or T7, preferably a T7 promoter. Preferably, the vector is suitable for in vitro transcription using a phage based in vitro transcription system, such as a T7 RNA polymerase based in vitro transcription system.

In some embodiments, the vector is designed for transcription of the artificial nucleic acid upon transfection into an eukaryotic cell, preferably upon transfection into a mammalian cell, or upon administration to a subject, preferably as described herein. In a preferred embodiment, the vector is designed for transcription of the artificial nucleic acid by an eukaryotic RNA polymerase, preferably RNA polymerase II or III, more preferably RNA polymerase III. In certain embodiments, the vector may comprise a U6 snRNA promoter or a H1 promoter and, optionally, a selection marker, e.g. a reporter gene (such as GFP) or a resistance gene (such as a puromycin or a hygromycin resistance gene).

Cell Comprising the Artificial Nucleic Acid or the Vector

According to one aspect of the present invention, a cell is provided that comprises the artificial nucleic acid or the vector described herein. The cell may be any cell, such as a bacterial cell or a eukaryotic cell, preferably an insect cell, a plant cell, a vertebrate cell, such as a mammalian cell (e.g. a human cell or a murine cell). The cell may be, for example, used for replication of the vector of the present invention, for example, in a bacterial cell. Furthermore, the cell, preferably a eukaryotic cell, may be used for synthesis of the artificial nucleic acid molecule according to the present invention.

The cells according to the present invention are, for example, obtainable by standard nucleic acid transfer methods, such as standard transfection, transduction or transformation methods. The term ‘transfection’ as used herein generally refers to the introduction of nucleic acid molecules, such as DNA or RNA (e.g. mRNA) molecules, into cells, preferably into eukaryotic cells. In the context of the present invention, the term ‘transfection’ encompasses any method known to the skilled person for introducing nucleic acid molecules into cells, preferably into eukaryotic cells, e.g. into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In this context, the artificial nucleic acid or the vector as described herein may be introduced into the cell in a transient approach or in order to maintain the artificial nucleic acid or the vector stably in the cell (e.g. in a stable cell line).

Preferably, the cell is a mammalian cell, such as a cell of human subject, a domestic animal, a laboratory animal, such as a mouse or rat cell. Preferably, the cell is a human cell. The cell may be a cell of an established cell line, such as a CHO, BHK, 293T, COS-7, HELA, HEK, Jurkat cell line etc., or the cell may be a primary cell, such as a human dermal fibroblast (HDF) cell etc., preferably a cell isolated from an organism. In a preferred embodiment, the cell is an isolated cell of a mammalian subject, preferably of a human subject.

Composition Comprising the Artificial Nucleic Acid

In a further aspect, the present invention concerns a composition comprising the artificial nucleic acid, the vector or the cell as described herein and, optionally, an additional excipient, preferably a pharmaceutically acceptable excipient. The composition described herein is preferably a pharmaceutical composition. The composition described herein may be used in treatment or prophylaxis of a subject, such as in a gene therapy approach. Alternatively, the composition can also be used for diagnostic purposes or for laboratory use, e.g. in in vitro experiments.

Preferably, the composition further comprises one or more vehicles, diluents and/or excipients, which are preferably pharmaceutically acceptable. In the context of the present invention, a pharmaceutically acceptable vehicle typically includes a liquid or non-liquid basis for the composition described herein. In one embodiment, the composition is provided in liquid form. In this context, preferably, the vehicle is based on water, such as pyrogen-free water, isotonic saline or buffered (aqueous) solutions, e.g. phosphate, citrate etc. buffered solutions. The buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of mammalian cells due to osmosis or other concentration effects. Reference media are, for instance, liquids occurring in in vivo methods, such as blood, lymph, cytosolic liquids, or other body liquids, or e.g. liquids, which may be used as reference media in in vitro methods, such as common buffers or liquids. Such common buffers or liquids are known to a skilled person. Ringer-Lactate solution is particularly preferred as a liquid basis.

One or more compatible solid or liquid fillers or diluents or encapsulating compounds suitable for administration to a subject may be used as well for the inventive pharmaceutical composition. The term “compatible” as used herein preferably means that these components of the (pharmaceutical) composition are capable of being mixed with the artificial nucleic acid, the vector or the cells as defined herein in such a manner that no interaction occurs which would substantially reduce the pharmaceutical effectiveness of the composition under typical use conditions.

The composition according to the present invention may optionally further comprise one or more additional pharmaceutically active components. A pharmaceutically active component in this context is a compound that exhibits a therapeutic effect to heal, ameliorate or prevent a particular indication or disease. Such compounds include, without implying any limitation, peptides or proteins, nucleic acids, (therapeutically active) low molecular weight organic or inorganic compounds (molecular weight less than 5000, preferably less than 1000), sugars, antigens or antibodies, or other therapeutic agents already known in the prior art.

Furthermore, the composition may comprise a carrier for the artificial nucleic acid molecule or the vector. Such a carrier may be suitable for mediating dissolution in physiological acceptable liquids, transport and cellular uptake of the pharmaceutical active artificial nucleic acid molecule or the vector. Accordingly, such a carrier may be a component, which is suitable for depot and delivery of an artificial nucleic acid molecule or vector described herein. Such components may be, for example, cationic or polycationic carriers or compounds, which may serve as transfection or complexation agent. Particularly preferred transfection or complexation agents, in this context, are cationic or polycationic compounds,

The term ‘cationic compound’ typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4. Accordingly, a cationic compound may be any positively charged compound or polymer, preferably selected from a cationic peptide or protein or a cationic lipid, which is positively charged under physiological conditions, particularly under physiological conditions in vivo. A ‘cationic peptide or protein’ may contain at least one positively charged amino acid, or more than one positively charged amino acid, e.g. selected from Arg, His, Lys or Orn. Accordingly, ‘polycationic compounds’ are also within the scope exhibiting more than one positive charge under the conditions given.

The composition as described herein preferably comprises the artificial nucleic acid or the vector in naked form or in a complexed form. In a preferred embodiment, the composition comprises the artificial nucleic acid or the vector in the form of a nanoparticle, preferably a lipid nanoparticle or a liposome.

Kit

According to a further aspect, the invention relates to a kit or kit of parts comprising the artificial nucleic acid molecule, the vector, the cell, and/or the (pharmaceutical) composition according to the invention.

Preferably, the kit additionally comprises instructions for use, cells for transfection, a means for administration of the composition, a (pharmaceutically acceptable) carrier or vehicle and/or a (pharmaceutically acceptable) solution for dissolution or dilution of the artificial nucleic acid molecule, the vector, the cells or the composition. In preferred embodiments, the kit comprises the artificial nucleic acid or the vector described herein, either in liquid or in solid form (e.g. lyophilized), and a (pharmaceutically acceptable) vehicle for administration. For example, the kit may comprise the artificial nucleic acid or the vector and a vehicle (e.g. water, PBS, Ringer-Lactate or another suitable buffer), which are mixed prior to administration to a subject.

Use of the Artificial Nucleic Acid, the Vector, the Composition or the Cell

In a further aspect, the present invention concerns the use of the artificial nucleic acid, the vector, the composition or the cell described herein.

In particular, the invention comprises the use of the artificial nucleic acid, the vector, the composition or the cell for site-directed editing of a target RNA. Therein, the artificial nucleic acid, the vector, the composition or the cell described herein is preferably used to promote site-specific editing of a target RNA, preferably by specifically binding to the target RNA via the targeting sequence and by recruiting to the target site a deaminase as described herein. That reaction may take place in vitro or in vivo.

In a preferred embodiment, the artificial nucleic acid, the vector or the composition is administered or introduced into a cell comprising a target RNA to be edited. Said cell comprising a target RNA preferably further comprises a deaminase, preferably as described herein. Said deaminase is preferably an endogenous deaminase, more preferably an adenosine or a cytidine deaminase, or a recombinant deaminase (such as a tagged deaminase or a mutant deaminase, preferably as described herein), which is either stably expressed in said cell or introduced into said cell, preferably prior or concomitantly with the artificial nucleic acid, the vector or the composition. Alternatively, the cell comprising the artificial nucleic acid or the vector described herein is used for site-directed editing of a target RNA by bringing into contact the cell and the target RNA or by introducing the target RNA into the cell, e.g. by transfection, preferably as described herein.

In a further preferred embodiment, the invention provides a method for site-directed editing of a target RNA, which comprises contacting a target RNA with the artificial nucleic acid and which essentially comprises the steps as described herein with respect to the use of the artificial nucleic acid, the vector, the composition or the cell for site-directed editing of an RNA.

The editing reaction is preferably monitored or controlled by sequence analysis of the target RNA.

The use and the method described herein may further be employed for in vitro diagnosis of a disease or disorder. Therein, the disease or disorder is preferably selected from the group consisting of infectious diseases, tumour diseases, cardiovascular diseases, autoimmune diseases, allergies and neurological diseases or disorders.

Medical Use of the Artificial Nucleic Acid, the Vector, the Composition or the Cell

In a further aspect, the artificial nucleic acid, the vector, the composition, the cell or the kit described herein is provided for use as a medicament, e.g. in gene therapy. Preferably, the artificial nucleic acid, the vector, the composition, the cell or the kit described herein is provided for use in the treatment or prophylaxis of a disease or disorder selected from the group consisting of infectious diseases, tumour diseases, cardiovascular diseases, autoimmune diseases, allergies and neurological diseases or disorders. According to a preferred embodiment, the artificial nucleic acid, the vector, the composition, the cell or the kit described herein is provided for use as a medicament or for use in the treatment or prophylaxis of a disease or disorder, preferably as defined herein, wherein the use as a medicament or the treatment or prophylaxis comprises a step of site-directed editing of a target RNA.

In one aspect, the present invention further provides a method for treating a subject with a disease or a disorder, the method comprising administering an effective amount of the artificial nucleic acid, the vector, the composition or the cell described herein to the subject, wherein the disease or the disorder is preferably selected from the group consisting of infectious diseases, tumour diseases, cardiovascular diseases, autoimmune diseases, allergies and neurological diseases or disorders.

The artificial nucleic acid, the vector, the cell, or the (pharmaceutical) composition described herein may be administered orally, parenterally, by inhalation spray, topically, rectally, nasally, buccally, vaginally, via an implanted reservoir or via jet injection. The term parenteral as used herein includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, and sublingual injection or infusion techniques. In a preferred embodiment, the artificial nucleic acid molecule, the vector, the cell or the (pharmaceutical) composition described herein is administered via needle-free injection (e.g. jet injection).

Preferably, the artificial nucleic acid, the vector, the cell, or the (pharmaceutical) composition described herein is administered parenterally, e.g. by parenteral injection, more preferably by subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, sublingual injection or via infusion techniques. Particularly preferred is intradermal and intramuscular injection. Sterile injectable forms of the inventive pharmaceutical composition may be aqueous or oleaginous suspension. These suspensions may be formulated according to techniques known in the art using suitable dispersing or wetting agents and suspending agents.

The artificial nucleic acid, the vector, the cell, or the (pharmaceutical) composition described herein may also be administered orally in any orally acceptable dosage form including, but not limited to, capsules, tablets, aqueous suspensions or solutions.

The artificial nucleic acid, the vector, the cell, or the (pharmaceutical) composition described herein may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the artificial nucleic acid, the vector, the cell, or the (pharmaceutical) composition described herein may be formulated in a suitable ointment suspended or dissolved in one or more carriers.

In one embodiment, the use as a medicament comprises the step of transfection of mammalian cells, preferably in vitro or ex vivo transfection of mammalian cells, more preferably in vitro transfection of isolated cells of a subject to be treated by the medicament. If the use comprises the in vitro transfection of isolated cells, the use as a medicament may further comprise the re-administration of the transfected cells to the patient. The use of the artificial nucleic acid or the vector as a medicament may further comprise the step of selection of successfully transfected isolated cells. Thus, it may be beneficial if the vector further comprises a selection marker.

According to another aspect of the present invention, the artificial nucleic acid, the vector, the cell, or the (pharmaceutical) composition described herein is provided for use in the diagnosis of a disease or disorder, which is preferably selected from the group consisting of infectious diseases, tumour diseases, cardiovascular diseases, autoimmune diseases, allergies and neurological diseases or disorders.

### BRIEF DESCRIPTION OF THE FIGURES

The figures shown in the following are merely illustrative and shall describe the present invention in a further way. These figures shall not be construed to limit the present invention thereto.

FIG. 1: Editing in engineered ADAR-expressing cell lines (293 Flp-In T-REx). A) Sequences and chemical modification patterns of several ASO designs. B) Initial sequence screening with plasmid-encoded guideRNAs by editing of a luciferase reporter. C) Comparative editing of two endogenous transcripts (ACTB, GAPDH) by transfection of the respective chemically modified ASOs into the indicated ADAR-expressing cell line. Either a single ASO (against GAPDH or ACTB) or two ASOs (against GAPDH and ACTB) were transfected. Data in B) are shown as the mean±SD, N=2 independent experiments. Data in C) are shown as the mean±SD, N=3 independent experiments. A1p110=ADAR1P110; A1p150=ADAR1p150.

FIG. 2: Editing of endogenous transcripts (GAPDH, ACTB, each 5′-UAG triplet in the 3′-UTR) by recruitment of endogenous ADARs in various cells and cell lines by transfection with various ASOs. Experiments were performed in presence or absence of IFN-α, as indicated.
A) Comparing three different ASOs for the recruitment of endogenous ADAR in HeLa cells. Either a single guideRNA (against GAPDH or ACTB) or both guideRNAs (against GAPDH and ACTB) were transfected. “no R/G” means an ASO lacking the ADAR recruiting domain. B) Comparative editing of guideRNA v9.4 and v9.5 on GAPDH. C) Effect of isoform-specific ADAR knockdown on the GADPH editing yield in HeLa cells. D) The knock-down efficiency was verified by Western blot. E) Determination of the effective dose (ED50) of ASO v9.5 for editing GAPDH in HeLa cells in a 96-well format. ED50=0.2 pmol/well (with IFN-α) and 0.4 pmol/well (without IFN-α). F) Time-course of GAPDH editing yields in HeLa cells with and without IFN-α. G) GAPDH editing yields with 5 pmol/96 well (25 pmol/24 well for SH-SYSY) ASO v9.5 in various standard (cancer) cells lines. H) GAPDH editing yields with ASO v9.5 (25 pmol/24 well, if not indicated differently) in various primary human cells. HUVEC=human umbilical vein endothelial cells; HAEC=human aortic endothelial cells; NHA=normal human astrocytes; RPE=human retinal pigment epithelium; NHBE=normal human bronchial epithelium. A-H) Data are shown as the mean±SD, N=3 independent experiments, experiments in hepatocytes are single determinations for each donor or an average (mean±SD) of the indicated donors. A1p150=ADAR1p150.

FIG. 3: ORF editing in primary cells and applications.
A) Editing of 5′-UAG codons in the ORF (site #2) versus 3′-UTR of endogenous GAPDH in 293-Flp-ln cells expressing the respective ADAR isoform applying ASO v9.4. B) ASO design v25 for ORF editing. C) Editing of a 5′-UAG site (#1) in the GAPDH ORF with ASO v25 in HeLa and primary cells. D) Editing of the Tyr701 site (5′-UAU codon) of STAT1 in HeLa and primary cells. E) Editing of the PiZZ mutation (E342K in SERPINA1, 5′-CAA codon) in ADAR1p150-expressing 293 cells, and in HeLa cells. SERPINA1 E342K cDNA was either co-transfected or genetically integrated into HeLa cells. A1AT secretion was data are shown as the mean±SD, N=3 independent experiments; experiments in hepatocytes are single determinations for each donor; n.d.=no editing was detectable.

FIG. 4: Editing yields for the editing of a 5′-UAG codon in the ORF of GAPDH in HeLa cells with ASO v25 containing a chemically unmodified versus modified ADAR recruiting domain.
ASO v25 with a chemically unmodified ADAR recruiting domain (unmod R/G), was compared to ASO of the same sequence with additional chemical modification (all pyrimidine nucleotides in the ADAR recruiting domain are backbone 2′-O-methylated). ASOs were transfected in HeLa cells. Data are shown as the mean±SD, N=3 independent experiments.

FIG. 5: Preferred embodiments of ASOs according to the invention.
A) General architecture of targeting sequence and recruiting moiety. Shown are different sequence variations of the recruiting moiety and different architectures of the targeting sequence. B) Exemplary modification patterns of the targeting sequence and the recruiting moiety, respectively.

FIG. 6: Serum stability of unmodified and modified ASOs.
A) Serum stability of a guideRNA targeting the codon 5′-AAA. GuideRNAs having a modified (2′-O-methyl or 2′-fluoro) nucleotide at the 5′ position of the anticodon were compared with the respective unmodified guideRNA. FIG. 2A shows an urea PAGE gel after incubation of the guideRNAs for from 5 minutes to 12 hours (see Example 5). B) Influence of the targeted codon on serum stability. C) Influence of the modification pattern of the targeting anticodon (3′-ACC) on serum stability.

FIG. 7: Site-directed RNA editing by SNAP-tagged ADARs driven by short, chemically modified guideRNAs.
a) The double-stranded RNA-binding domains (dsRBDs) of hADAR have been substituted with the SNAP-tag. The latter is able to form a covalent bond to a guideRNA that is modified with benzylguanine (BG). When bound to the SNAP-ADAR, the guideRNA targets the attached SNAP-ADAR protein to the catalyzed by the deaminase domain. b) A typical BG-guideRNA that targets a UAG site with a 5′-CCA anticodon. The guideRNA is 22-nt long and is densely chemically stabilized by 2′-methoxylation and terminal phosphorothioate linkages. The first three 5′-terminal nucleotides do not base pair with the target RNA, but serve as a linker. The sequence preferably comprises an unmodified or partially modified ribonucleotide gap (5′-CCA) which faces the target site and contains a central mismatching cytosine opposite the targeted adenosine for efficient deamination. A C6-amino-linker is located at the 5′-end of the guideRNA to introduce the BG modification to the full length oligonucleotide. c) Experimental setup. Cells with stably integrated SNAP-ADAR (SA) are seeded into 24-well plates with medium containing doxycycline (dox) to induce SA expression. 24 h later, the cells were reverse-transfected with the guideRNA. After 24 h, the cells were lysed for RNA isolation to analyze RNA editing.

FIG. 8: Editing performance of four SNAP-ADARs. a) Engineered 293 cell lines expressing the respective SA enzyme were transfected with either a single gRNA or 4 gRNAs against 5′-UAG triplets in the indicated endogenous transcripts. b), c) Time- and dose-dependency of editing in the GAPDH transcript. d) Editing of 5′-UAG sites in various transcripts, 5′-UTR versus ORF and 3′-UTR. e) Comparative editing of all 16 triplets (5′-NAN) in the ORF of the endogenous GAPDH transcript. a)-e) Data are shown as the mean±SD, N=3 independent experiments, black clots represent individual data points.

FIG. 9: Controlling off-target editing in SAQ cells.
a) In order to avoid unintended editing of an adjacent adenosine at the target site, the opposing base in the guideRNA can be modified by 2′-methoxylation (M) or 2′-fluorination (F). This is exemplary shown for the triplet CAA. b) Off-target editing of an adjacent adenosine was detected in the triplets CAA, AAA, AAC and UAA when particularly using SA2Q cells. However, off-target editing was remarkably reduced when the strategy was applied. Data are shown as the mean±SD, N=3 independent experiments, black dots represent individual data points.

FIG. 10: Effect of chemical modification on editing yields and serum stabilities. Examples of chemical modifications that stabilize the 3′-ACC anticodon (A) and the 3′-UCC anticodon (B), respectively, in the targeting sequence, e.g. 2′-F, 2′-O-methyl, 2′-deoxy and by phosporthioate modification.

FIG. 11: Conjugation of branched and multiple copies of the coupling agents to guideRNAs. Shown are schemes for the coupling of 1×BG, 2×BG, or 4×BG either to one terminus or to two sites at an ASO. Those architectures allow for recruiting several deaminases to the target, clearly improving their editing performance, e.g. with respect to potency (see FIG. 12).

FIG. 12: Application of branched/multiple coupling agents results.
Various guideRNAs having an architecture as shown in FIG. 11 have been tested for the editing of the Tyr701 codon in the endogenous STAT1 transcript in 293-Flp-In cells expressing SNAP-ADAR1Q. Specifically, we applied guideRNAs that contained either a 5′-amino linker or both, a 5′- and a 3′-amino linker and linked them to one (single) or two (double) of the coupling agents (1×BG, 2×BG, or 4×BG), respectively.

### EXAMPLES

The examples shown in the following are merely illustrative and shall describe the present invention in a further way. These examples shall not be construed to limit the present invention thereto.

### Example 1

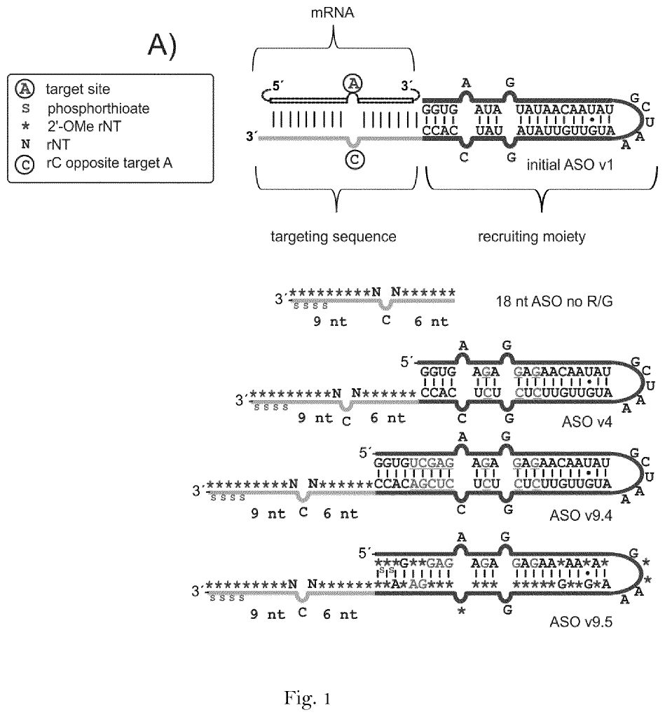

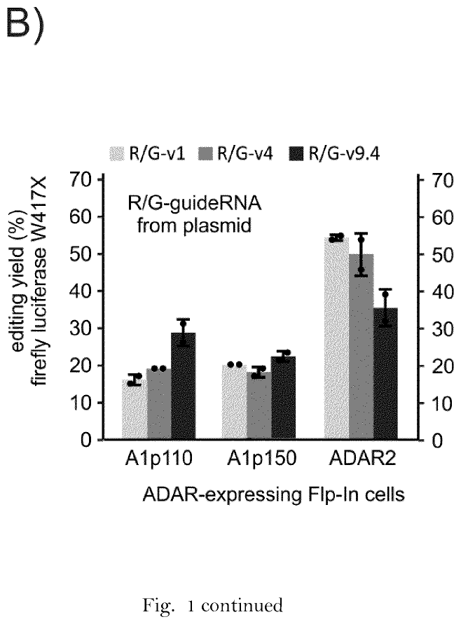

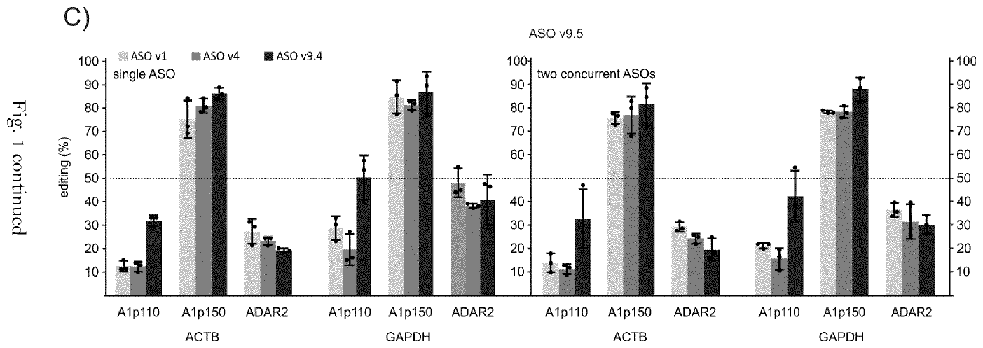

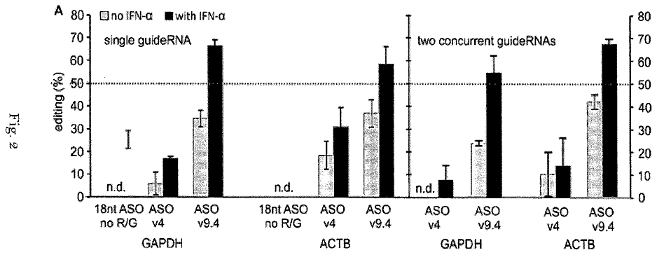

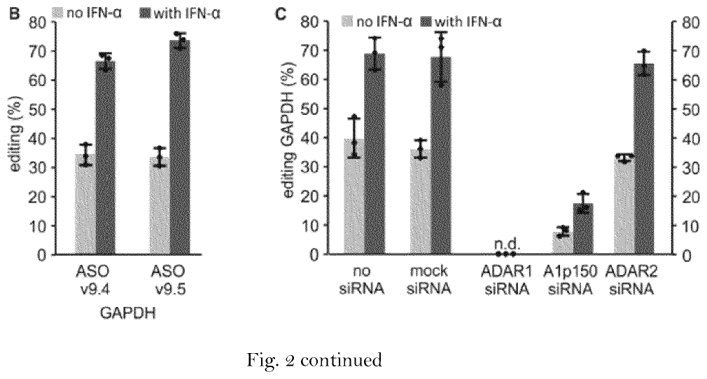

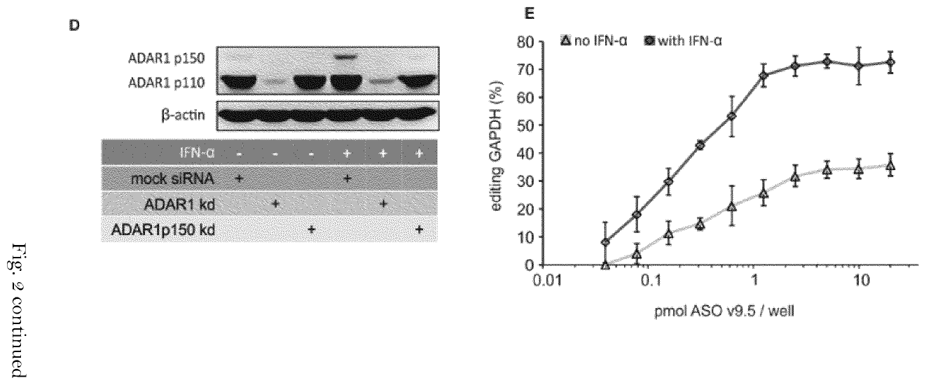

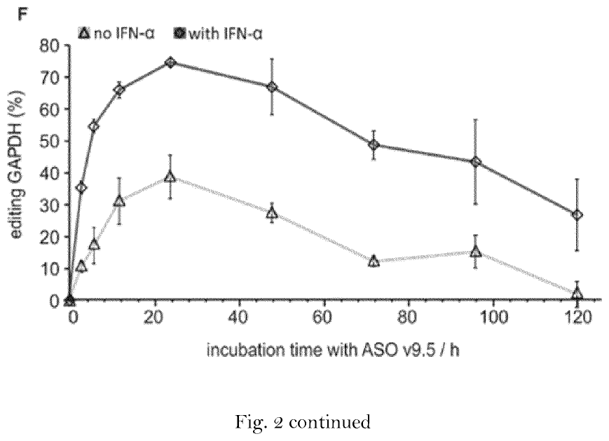

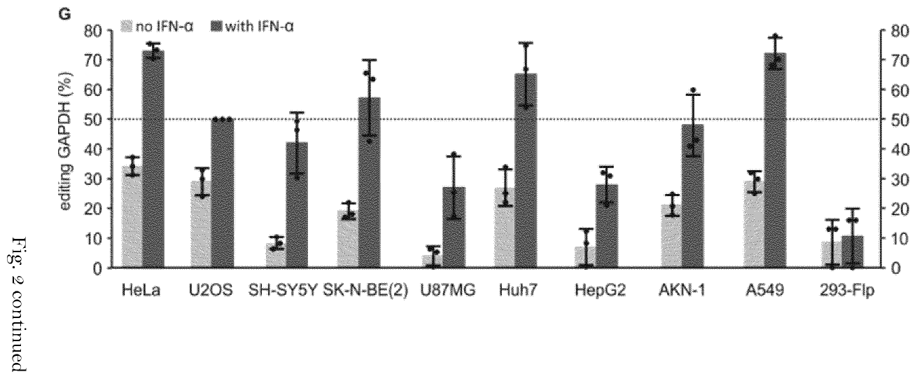

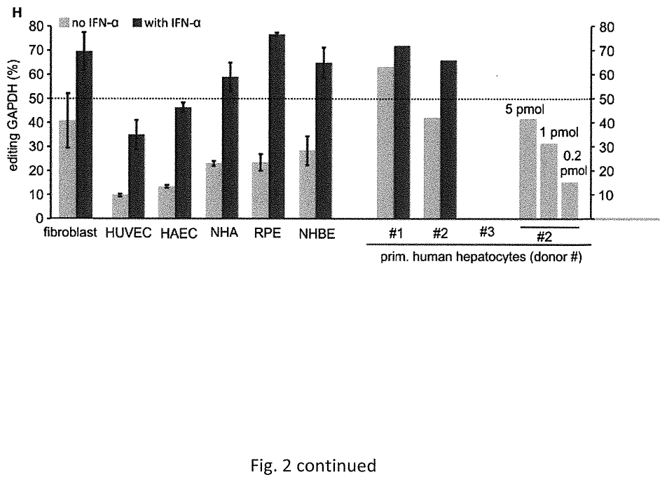

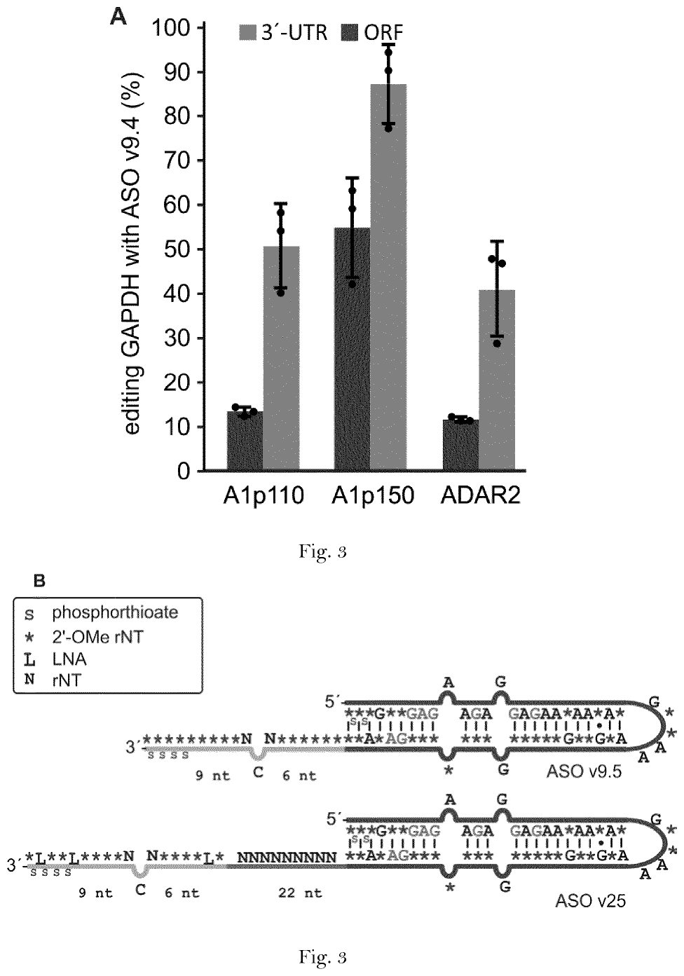

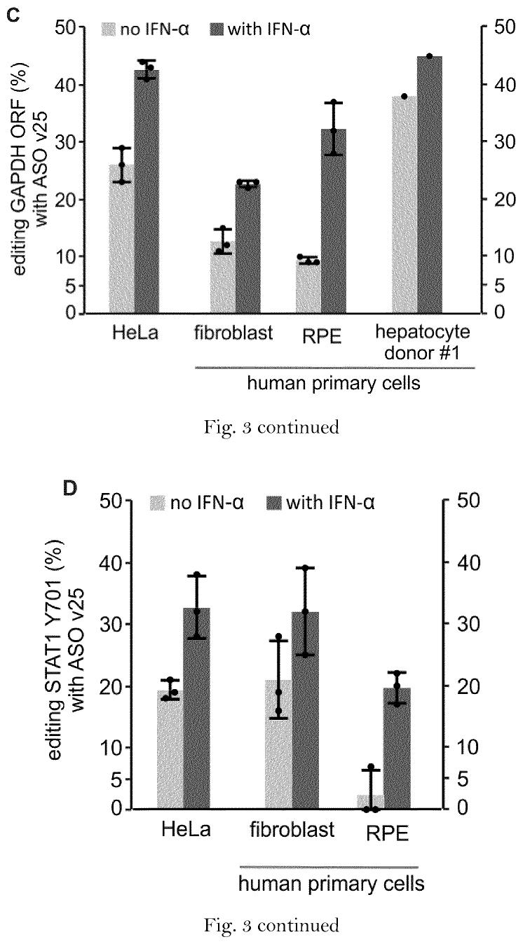

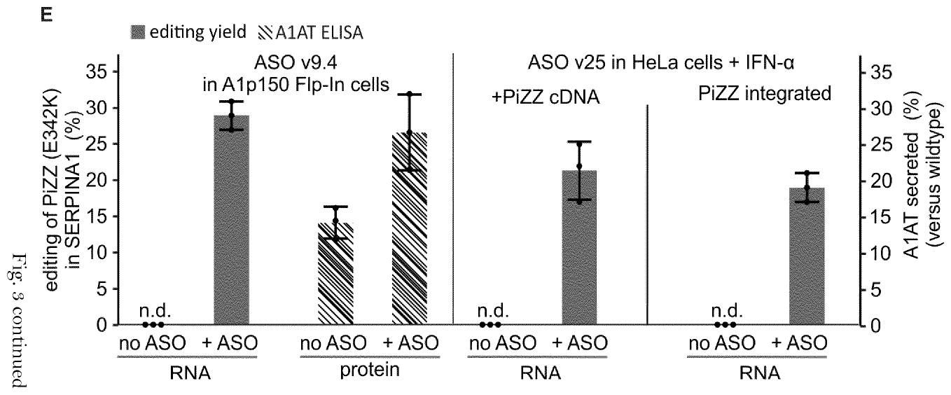

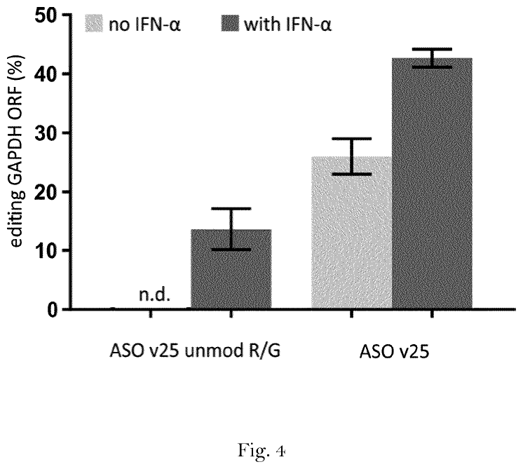

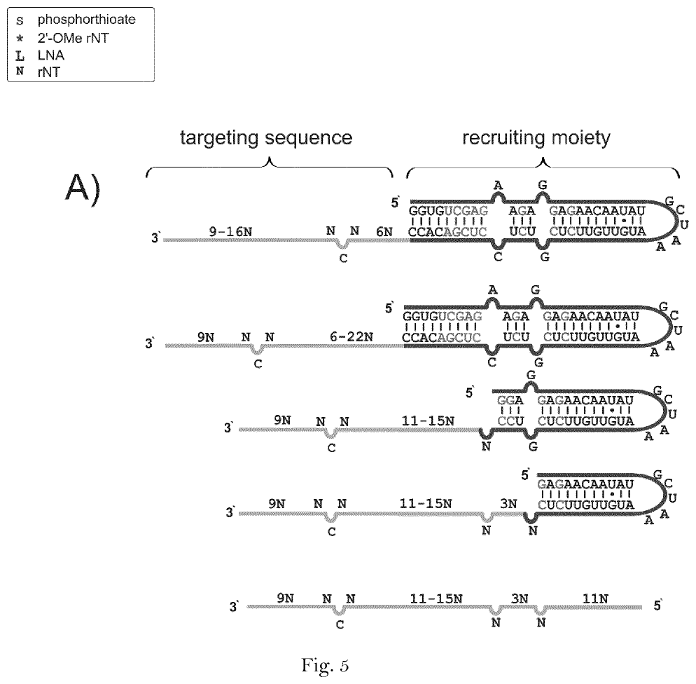

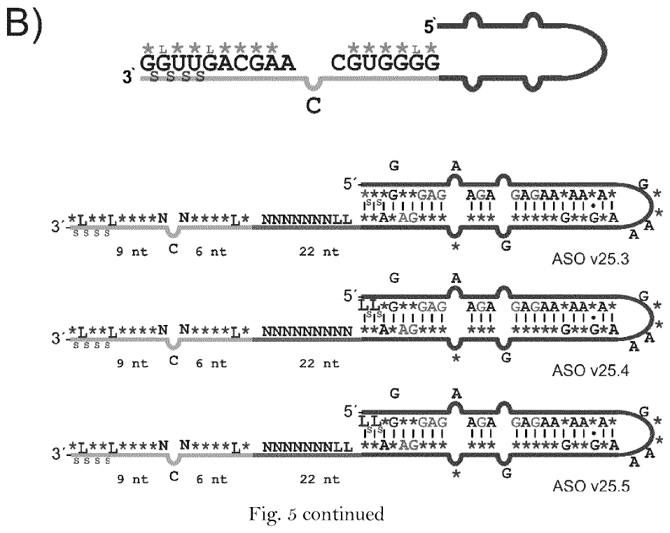

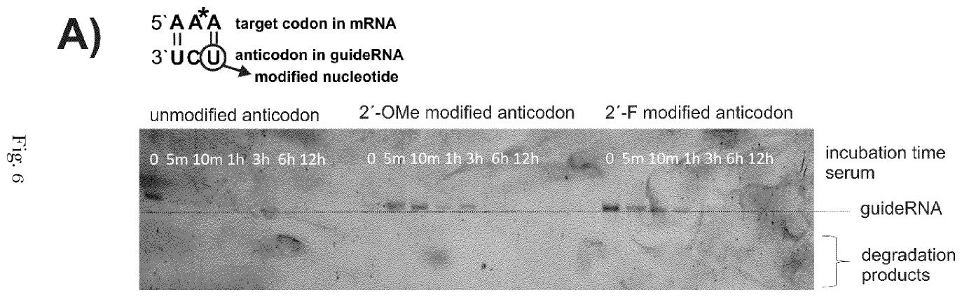

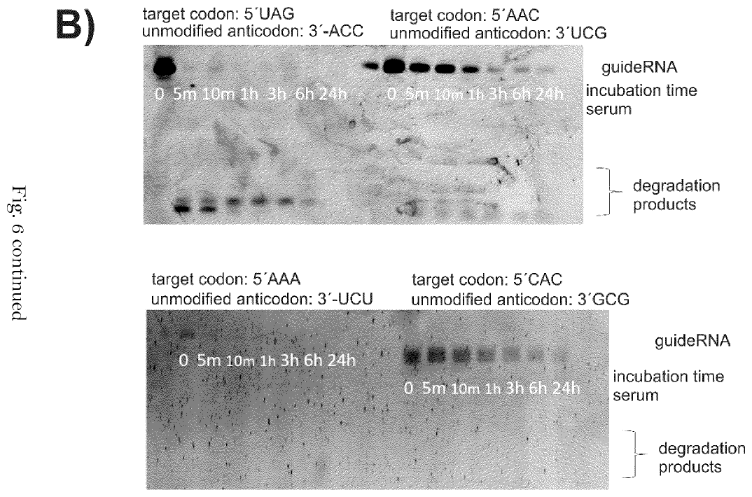

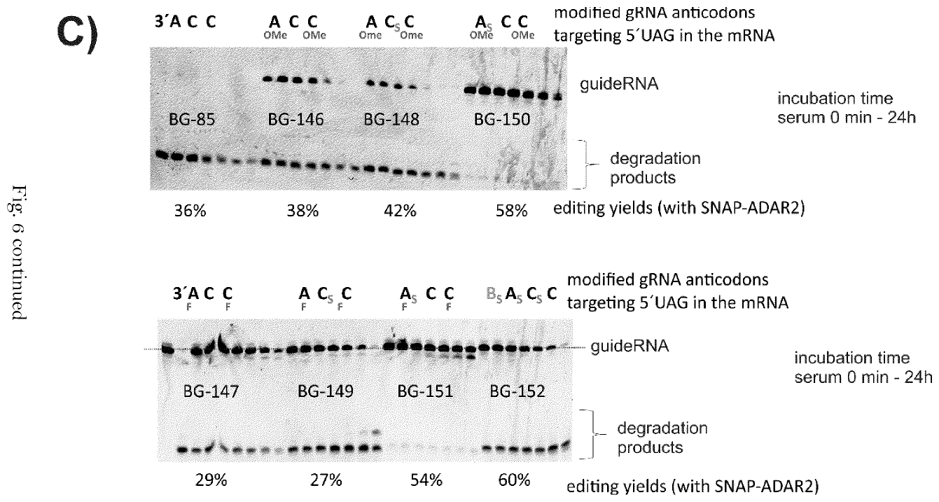

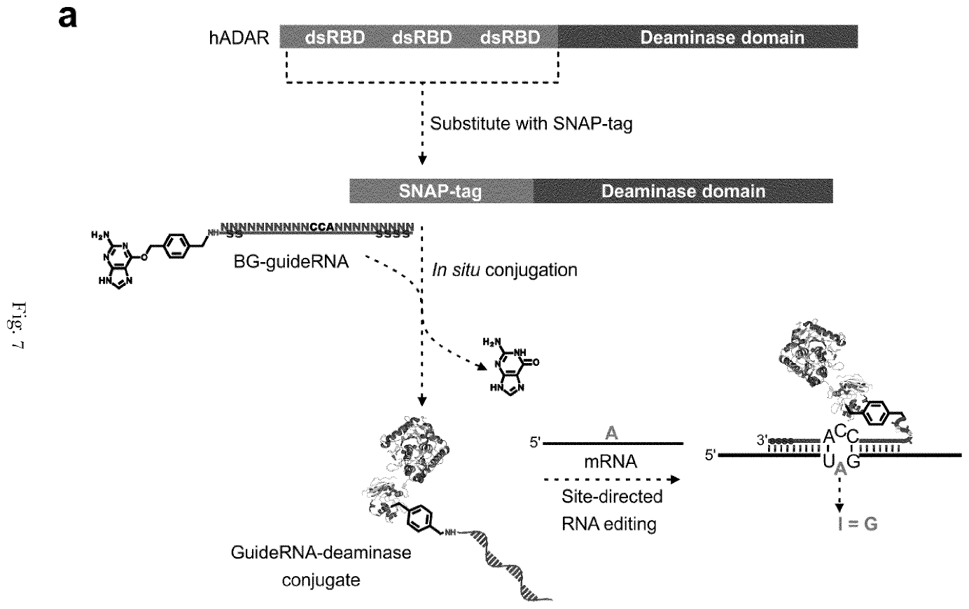

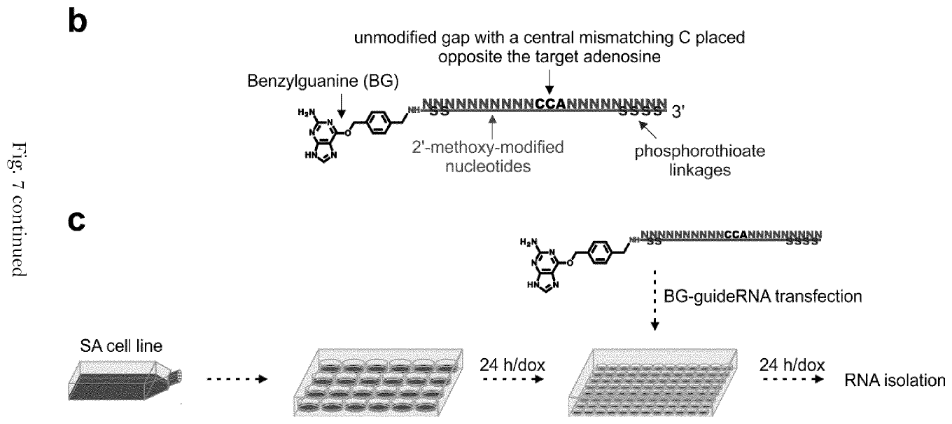

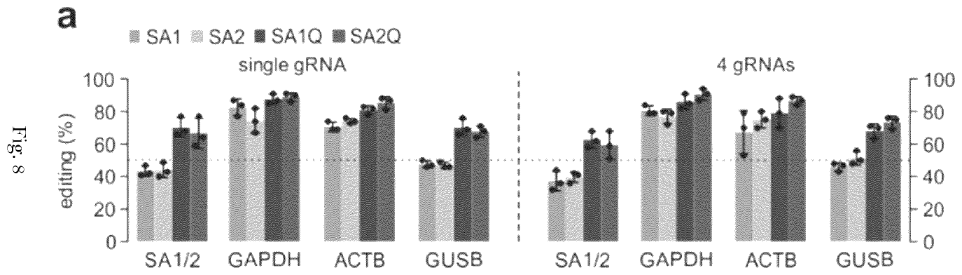

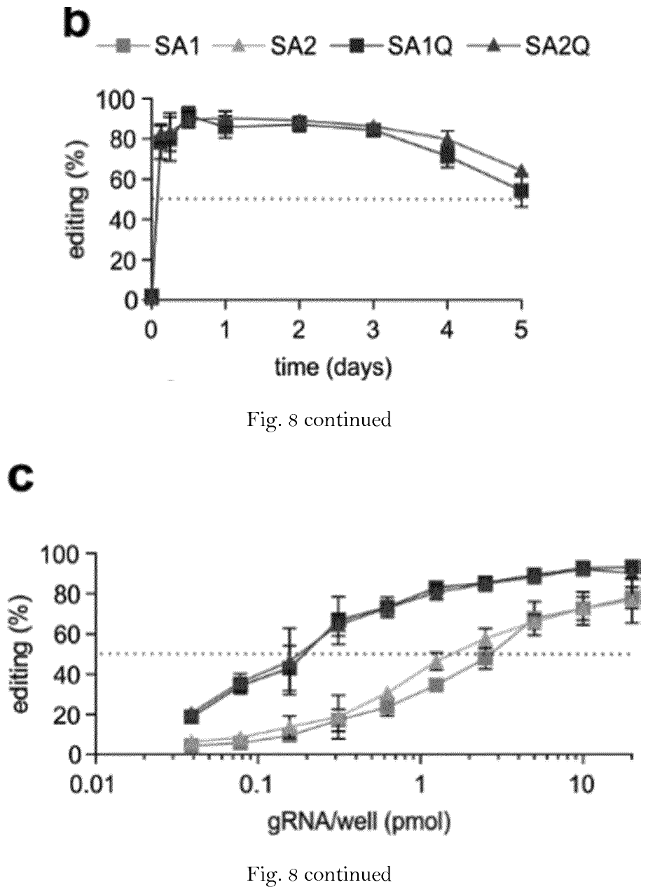

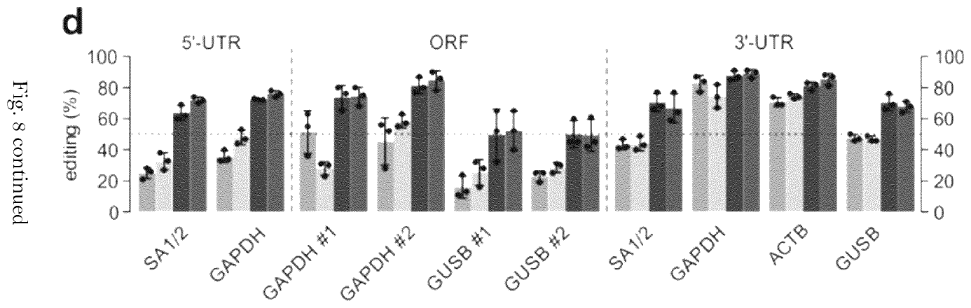

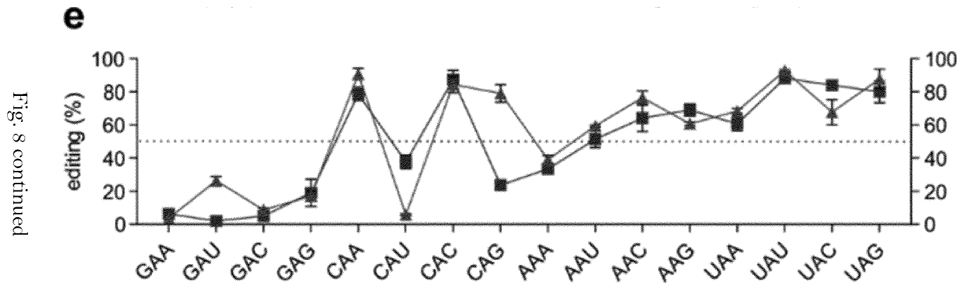

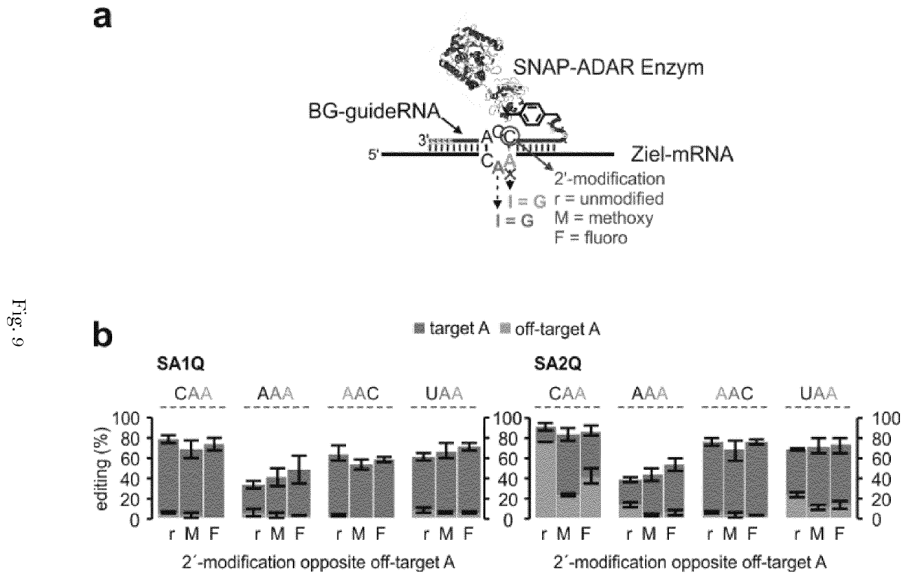

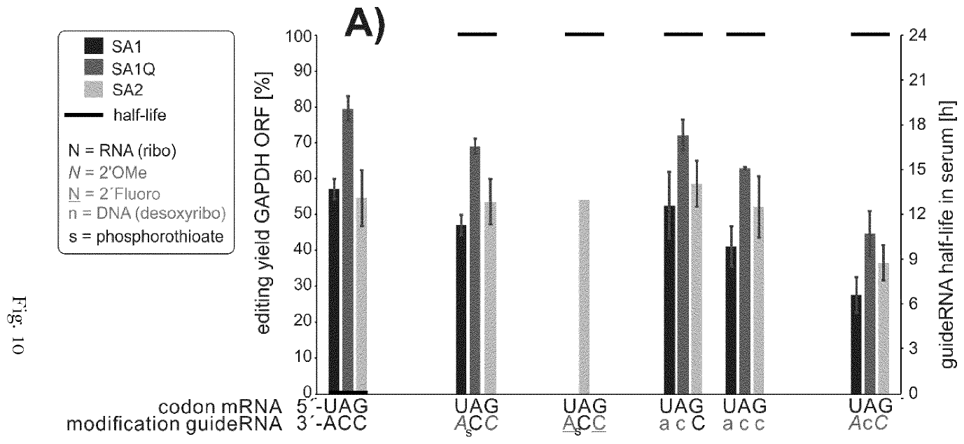

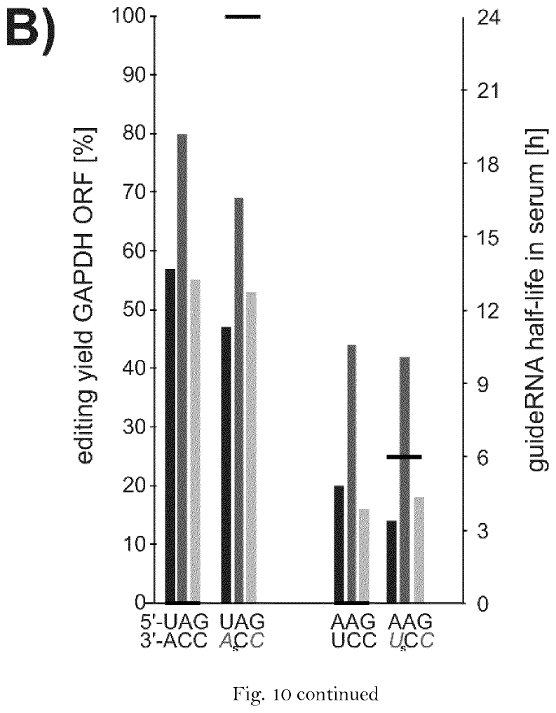

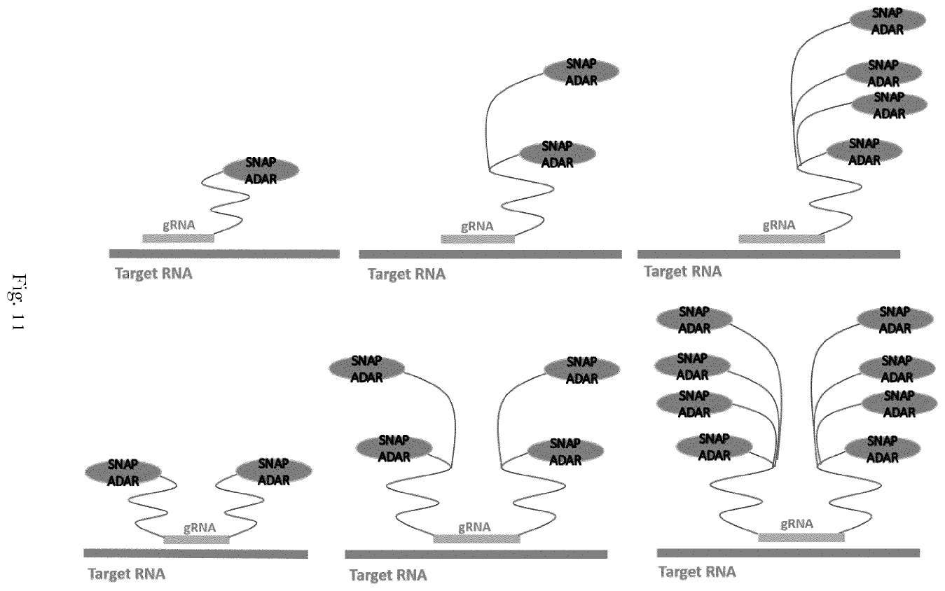

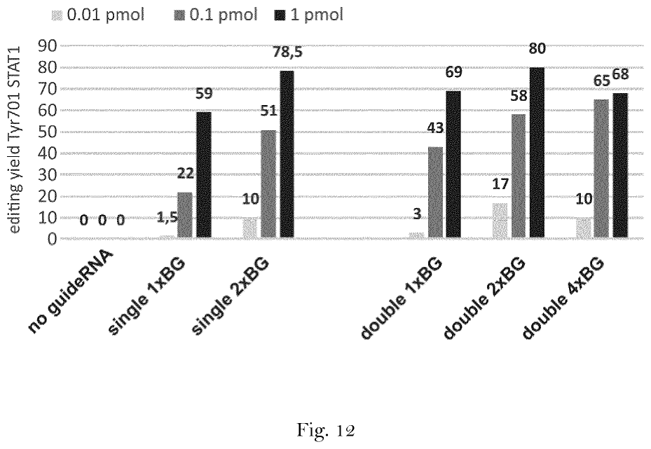

Unmodified RNA oligonucleotides were produced by in vitro transcription from linear synthetic DNA templates (purchased from Sigma-Aldrich, Germany) with T7 RNA polymerase (Thermo Scientific, USA) at 37° C. overnight. The resulting RNA was precipitated in ethanol and purified via urea (7M) polyacrylamide (15%) gel electrophoresis (PAGE), extracted into water, precipitated with ethanol and resuspended and stored in nuclease-free water. All chemically modified RNA oligonucleotides purchased from Biospring (Germany), Eurogentec (Belgium) or Dharmacon (USA). Long sequences were assembled from two pieces by ligation. As a first step, a plasmid-borne approach was applied in order to screen for suitable guideRNA sequences. A reporter editing assay (FIG. 1B), led to the identification of sequence variant 9.4 that has additional 5 bp at the 5′-site of the RNA helix in the ADAR recruiting domain.

In the reporter editing assay, Firefly luciferase was expressed under control of a CMV promotor from a pShuttle-CMV plasmid. The W417X amber mutation was introduced via overlap PCR. Sequences of the cloned products were determined by Sanger sequencing. The RIG-guideRNAs were expressed under control of the U6 promotor from a modified pSilencer backbone similar as described in Wettengel et al. (Wettengel, J., Reautschnig, J., Geisler, S., Kahle, P. J., Stafforst, T. Harnessing human ADAR2 for RNA repair—Recoding a PINK1 mutation rescues mitophagy. Nucl. Acids Res. 45, 2797-2808 (2017). Sequences of the cloned products were determined by Sanger sequencing. Sequences of the applied R/G-guideRNAs are provided in Table 1.

TABLE 1
R/G guideRNAs

| R/G guide RNAs | |
|---|---|
| expressed from | |
| plasmid | 5′-3′ sequence |
| Luciferase R/G-v1 | (GUGGAAUAGUAUAACAAUAU |
| | GCUAAAUGUUGUUAUAGUAUC |
| | CCACGUGCAGCCAGCCGUCCU |
| | CUAGAGGGCCCUGAAGAGGGC |
| | CC) (SEQ ID NO: 61) |
| Luciferase R/G-v4 | (GUGGAAGAGGAGAACAAUAU |
| | GCUAAAUGUUGUUCUCGUCUC |
| | CCACGUGCAGCCAGCCGUCCU |
| | CUAGAGGGCCCUGAAGAGGGC |
| | CC) (SEQ ID NO: 62) |
| Luciferase R/G-v9.4 | (GUGGUCGAGAAGAGGAGAAC |
| | AAUAUGCUAAAUGUUGUUCUC |
| | GUCUCCUCGACCACGUGCAGC |
| | CAGCCGUCCUCUAGAGGGCCC |
| | UGAAGAGGGCCC) |
| | (SEQ ID NO: 63) |
| Chemically | |
| synthesized ASOs | 5′-3′ sequence |
| ACTB 3′UTR 18 nt | [GC AAU G](CCA) [UC AC] |
| | [C*][U*][C*][C*][C] |
| | (SEQ ID NO: 64) |
| ACTB 3′UTR ASO | (GGUGA AUAGUAUAAC AAUAU |
| v1 | GCUAA AUGUUGUUAUAGUAUCC |
| | ACC) [GC AAU G](CCA) |
| | [UC AC][C*][U*][C*] |
| | [C*][C](SEQ ID NO: 65) |
| ACTB 3′UTR ASO | (GGUGAAG AGGAGAACAA |
| v4 | UAUGCUAAAU GUUGUUCUC |
| | GUCUCCACC)[GC AAU G] |
| | (CCA) [UC AC][C*][U*] |
| | [C*][C*][C] |
| | (SEQ ID NO: 66) |
| ACTB 3′UTR ASO | (GGU GUC GAG AAG AGG |
| v9.4 | AGA AC AAU AUG CUA AAU |
| | GUUGUU CUCGUC UCC UCG |
| | ACA CC) [GC AAU G] |
| | (CCA) [UC AC][C*][U*] |
| | [C*][C*][C] |
| | (SEQ ID NO: 67) |
| GAPDH 3′UTR 18 nt | [AG GGG U](CCA) |
| | [CA UG][G*][C*] |
| | [A*][A*][C] |
| | (SEQ ID NO: 68) |
| GAPDH 3′UTR | (GGUGA AUAGUAUAAC |
| ASO v1 | AAUAUGCUAA AUGUUGU |
| | UAUAGUAUCCACC) |
| | [AG GGG U](CCA) |
| | [CA UG][G*][C*] |
| | [A*][A*][C] |
| | (SEQ ID NO: 69) |
| GAPDH 3′UTR | (GGUGAAG AGGAGAACAA UAUGCUAAAU |
| ASO v4 | GUUGUUCUCGUCUCCACC)[AG GGG U] |
| | (CCA)[CA UG][G*][C*][A*][A*] |
| | [C](SEQID NO: 70) |
| GAPDH 3′UTR | (GGU GUC GAG AAG AGG AGA AC |
| ASO v9.4 | AAU AUG CUA AAU GUUGUU |
| | CUCGUC UCC UCG ACA CC) |
| | [AG GGG U](CCA) [CA UG] |
| | [G*][C*][A*][A*][C] |
| | (SEQ ID NO: 71) |
| GAPDH 3′UTR | [G*][G*][U](G)[U][C] |
| ASO v9.5 | (GAG AAG AGG AGA A)[C] |
| | (AA)[U](A)[U](G) [C][U] |
| | (A AA)[U](G)[UU](G) |
| | [UUCUC](G)[UCUCCUC](GA) |
| | [C](A) [CCAGGGGU](CCA) |
| | [CAUG][G*][C*][A*][A*] |
| | [C](SEQ ID NO: 72) |
| GAPDH ORF1 ASO | [GGG GUG](CCA) [AG CA] |
| 18 nt | [G*][U*][U*][G*][G] |
| | (SEQ ID NO: 73) |
| GAPDH ORF1 ASO | (GGU GUC GAG AAG AGG AGA |
| v9.4 | AC AAU AUG CUA AAU GUUGUU |
| | CUCGUC UCC UCG ACA CC) |
| | [GGG GUG](CCA)[AG CA][G*] |
| | [U*][U*][G*][G] |
| | (SEQ ID NO: 74) |
| GAPDH ORF2 ASO | [GGG GUG](CCA)[AG CA] |
| 18 nt | [G*][U*][U*][G*][G] |
| | (SEQ ID NO: 75) |
| GAPDH ORF2 ASO | (GGU GUC GAG AAG AGG AGA |
| v9.4 | AC AAU AUG CUA AAU GUUGUU |
| | CUCGUC UCC UCG ACA CC) |
| | [GU UUU U](CCA) [GA CG] |
| | [G*][C*][A*][G*][G] |
| | (SEQ ID NO: 76) |
| GAPDH ORF1 ASO | [G]*[G]*[U](G)[U][C] |
| v25 | (GAG AAG AGG AGA A)[C] |
| | (AA) [U](A)[U](G) [C] |
| | [U](A AA)[U](G)[U][U] |
| | (G)[U][U][C][U][C](G) |
| | [U][C][U][C][C][U][C] |
| | (G A)[C](A) [C][C] |
| | (UUGUCAUGGAUGACCUUGGCCA) |
| | [G]{G}[GG UG](CCA) |
| | [AGCA]{G*}[U*][U*]{G*} |
| | [G](SEQ ID NO: 77) |
| GAPDH ORF1 ASO | [G]*[G]*[U](GUCGAG AAG |
| R/G unmod v25 | AGG AGA ACAAUAUGCUAAAUG |
| | UUGUUCUCGUCUCCUCG ACACC |
| | UUGUCAUGGAUGACCUU GGCCA) |
| | [G][G][GG UG](CCA) |
| | [AGCA][G*][U*][U*]{G*} |
| | [G](SEQ ID NO: 78) |
| SERPINA ASO v9.4 | (GGU GUC GAG AAG AGG AGA |
| | AC AAU AUG CUA AAU GUUGUU |
| | CUCGUC UCC UCG ACA CC) |
| | [CCU UUC](UCG) [UCG A] |
| | [U*][G*][G*][U*][C] |
| | (SEQ ID NO: 79) |
| SERPINA ASO 40 nt | (CAUGGCCCCAGCAGCUUCAGUC) |
| | [C]{C}[UUUC](UCG)[UCGA] |
| | {T*}[G*][G*]{T*}[C] |
| | (SEQ ID NO: 80) |
| SERPINA ASO v25 | [G*][G*][U](G)[U][C] |
| | (GAG AAG AGG AGA A)[C] |
| | (AA) [U](A)[U](G) [C] |
| | [U](A AA)[U](G)[U][U] |
| | (G)[U][U][C][U][C](G) |
| | [U][C][U][C][C][U][C] |
| | (G ACACC CAUGGCCCCAGCA |
| | GCUUCAGUC)[C]{C}[UUUC] |
| | (UCG) [UCGA]{T*}[G*] |
| | [G*]{T*}[C] |
| | (SEQ ID NO: 81) |
| SERPINA ASO v25 | [G*][G*][U](G)[U][C] |
| | (GAG AAG AGG AGA A)[C] |
| | (AA) [U](A)[U](G) [C] |
| | [U](A AA)[U](G)[U][U] |
| | (G)[U][U][C][U][C](G) |
| | [U][C][U][C][C][U][C] |
| | (G ACACC CAUGGCCCCAGCA |
| | GCUUCAGUC)[C]{C}[UUUCU] |
| | (CG) [UCGA][T*][G*] |
| | [G*]{T*}[C] |
| | (SEQ ID NO: 82) |
| STAT1 ASO v25 | [G*][G*][U](G)[U][C] |
| | (GAG AAG AGG AGA A)[C] |
| | (AA) [U](A)[U](G) [C] |
| | [U](A AA)[U](G)[U][U] |
| | (G)[U][U][C][U][C] |
| | (G)[U][C][U][C] |
| | [C][U][C](GACACCCAG |
| | ACACAGAAAUCAACUCAGU) |
| | [C][T][UGAU](ACA) |
| | [UCCA]{G*}[U*][U*] |
| | {C*}[C] |
| | (SEQ ID NO: 83) |
| GAPDH 3′UTR | (GGUGA AUAGUAUAAC |
| unmod ASO v1 | AAUAUGCUAA AUGUUGUUA |
| | UAGUAUCCACC AG GGG |
| | UCCACA UG GCAAC) |
| | (SEQ ID NO: 84) |
| GAPDH 3′UTR | (GGUGAAG AGGAGAACAA UAU |
| unmod ASO v4 | GCUAAAU GUUGUUCUCGUCUCC |
| | ACCAG GGG UCCACA UGGCAAC) |
| | (SEQ ID NO: 85) |
| GAPDH 3′UTR | (GGU GUC GAG AAG AGG AGA |
| unmod ASO v9.4 | AC AAU AUG CUA AAU |
| | GUUGUU CUCGUC UCC UCG |
| | ACA CCAG GGG U CCACAU |
| | GGCAAC) (SEQ ID NO: 86) |
| Sense guideRNAs | |
| for RT PCR | 5′-3′ sequence |
| GAPDH sense | (GGACCAACUGCUUGGCACCCCUG |
| | GCCAAGGUCAUCCAUGACAACUUU |
| | GGUAUCGUGGAAGGACC) |
| | (SEQ ID NO: 87) |
| STAT1 sense | (GGGAACUGGAUCUAUCAAGACUG |
| | AGUUGAUUUCUGUGUCUGAAGUGU |
| | AAGUGAACACAGAA) |
| | (SEQ ID NO: 88) |
| SERPINA1 sense | (GGACCATCGACGAGAAAGGGACT |
| | GAAGCTGCTGGGGCCATGTTTTTA |
| | GAGGCCATACCCAT) |
| | (SEQ ID NO: 89) |

Legend to Table 1:(N) = RNA base, [N] = 2′-OMe RNA base, *= Phosphorothioate linkage, {N} = LNA base

Flp-In 293 T-REx cells (R78007, Thermo Fisher scientific) containing the respective gnomically integrated ADAR version were generated as described in Wettengel et al. and in Heep et al. (Heep, M., Mach, P., Reautschnig, P., Wettengel, J., Stafforst, T. Applying Human ADAR1p110 and ADAR1p150 for Site-Directed RNA Editing—G/C Substitution Stabilizes GuideRNAs Against Editing. Genes 8, 34 (2017)). Cells were cultured in DMEM+10% FBS+100 μg/ml hygromycin B+15 μg/ml blasticidin S. For editing, 2.5×10⁵ cells/well (ADAR1p110, ADAR1p150) or 3×10⁵ cells/well (ADAR2) were seeded into poly-D-lysine-coated 24-well plates in 500 μl DMEM+10% FBS+10 ng/ml doxycycline. Twenty-four hours later, transfection was performed with the luciferase reporter plasmid (300 ng) and the R/G-guideRNA (1300 ng) using a Lipofectamine-2000 to plasmid ratio of 3:1. The medium was changed every 24 h until harvest. RNA was isolated and sequenced 72 h post transfection, as described above.

Even though being less effective in recruiting ADAR2 (35% reduced editing yield), sequence variant 9.4 turned out to improve editing yield with ADAR1p110 by almost twofold.

In a next step, the plasmid-borne expression of the guideRNA was replaced by the administration of chemically stabilized antisense oligonucleotides (ASO). In the first round, three chemically stabilized ASO designs (v1, v9, v9.4) were tested for the editing of a respective 5′-UAG site in the 3′-UTR of GAPDH and ACTB. While the ADAR recruiting domain comprised of natural ribonucleotides, the 17 nt antisense part of the ASO was designed as an Antagomir-like modified gapmer10 (global 2′-Omethylation, 3′-terminal phosporthioate linkages, FIG. 1A) with a gap of three natural ribonucleosides opposite to the editing site, similar as described in Vogel et al. (Vogel, P., Schneider, M. F., Wettengel, J., Stafforst, T. Improving Site-Directed RNA Editing In Vitro and in Cell Culture by Chemical Modification of the GuideRNA. Angew. Chem. Int. Ed. 53, 6267-6271 (2014)) for the SNAP-ADAR approach. We constructed ASOs targeting a specific 5′-UAG site either in the 3′-UTR of the housekeeping gene ACTB or GAPDH.

To assess the individual ADAR preference of such ASOs, we lipofected them into engineered 293 Flp-In T-REx cells expressing a specific ADAR isoform (ADAR2, ADAR1p110 or ADAR1p150)11 under control of a CMV tet-on promotor. 48 Hours before ASO transfection, 2×10⁵ of the respective ADAR-Flp-In 293 T-REx cells per well were seeded in 24 well plates in DMEM+10% FBS containing 10 ng/mL doxycycline for induction of ADAR gene expression. After 48 hours cells were detached and reverse-transfected in 96 well plates. To this end, the respective ASO (5 pmol/well unless stated otherwise) and Lipofectamine 2000 (0.75 μL/well) were each diluted with OptiMEM to a volume of 10 μL in separate tubes, respectively. After 5 minutes, both solutions were mixed and 100 μL cell suspension (5×10⁴ cells) in DMEM+10% FBS+10 ng/mL doxycycline was added to the transfection mixture inside 96 wells. 24 hours later, cells were harvested for RNA isolation and sequencing, as described above.

Notably, particularly high editing yields (75-85%) were detected for both targets in ADAR1p150-expressing cells (FIG. 1C). The editing yields were lower for the ADAR1-isoform p110, ranging from 12-50%, however, the data clearly shows the strong (2-3fold) benefit in the editing yield for the new guideRNA sequence 9.4 compared to the initial version 1. Editing with ADAR2 stayed in a range comparable to that of ADAR1p110 (15-50%), again being less effective with the new design 9.4 compared to the old version 1. Finally, we tested the concurrent editing of both transcripts by co-transfection of both ASOs (FIG. 1C, right panel). The editing yields stayed virtually unchanged demonstrating that site directed RNA editing can potentially be carried out at several sites or transcripts simultaneously.

### Example 2

In a further series of experiments, endogenously expressed ADAR was harnessed for the editing of a 5′-UAG codon in the 3′-UTR of the two housekeeping genes GAPDH and ACTB in HeLa cells by simple lipofection of the respective ASOs.

To this end, HeLa cells (Cat. No.: ATCC CCL-2) were cultured in DMEM+10% FBS+P/S (100U/mL penicillin and 100 μg/mL streptomycin. 5×10⁴ cells in 100 μL DMEM+10% FBS 600 units IFN-α, Merck, catalog number IF007, lot number 2937858) were added to a transfection mix of 0.5 μL Lipofectamine 2000 and 5 pmol guideRNA/well in a 96-well format. For concurrent editing with two different ASOs, 2.5 pmol of each respective ASO were co-transfected. After 24 hours cells were harvested for RNA isolation and sequencing.

A control ASO comprising only of the specificity domain but lacking the ADAR recruiting domain did not elicit any editing (FIG. 1A, 2A). Some editing was observed with the initial sequence v1 and the design v4 (FIG. 2A). However, the new sequence v9.4 gave clearly higher editing in both transcripts with yields around 40%. In view of the circumstance that ASO design v9.4 worked particularly well with ADARp150, the experiment was repeated with HeLa cells that were pre-treated with IFN-α, which is known to induce ADAR1p150 expression. Indeed, IFN-α treatment almost doubled the editing yields for all ASO designs (v1, v4, v9.4) and for both transcripts to obtain editing yields up to 70%. The results confirmed that sequence v9.4 is superior in harnessing endogenously expressed ADARs.

Also in this series of experiments, editing of both transcripts was further analysed after simultaneous co-transfection of two guideRNAs. Also in this setting, the editing yields remained unchanged at high levels (FIG. 2A, right panel). In order to assess the influence of the chemical modification, the recruitment of overexpressed ADARs in Flp-In cells was also tested with unmodified, in-vitro transcribed guideRNAs of the same sequence, and was found to be clearly inferior compared to the chemically stabilized ASOs.

In a next step, the chemical modification was extended to the ADAR recruiting domain. Specifically, the 5′-terminus was stabilized by 2′-O-methylation and phosphorthioate linkages and all pyrimidines were substituted with their 2′-O-methylated analogs. Even though heavily modified, this ASO design v9.5 was equal or even better in recruiting endogenous ADAR in HeLa cells (FIG. 2B), demonstrating that ADARs′ dsRNA-binding domains accept extensive chemical modification.

In order to assess, which ADAR isoform was recruited by ASO v9.5 in HeLa cells, the expression of ADARs was determined in Western Blot experiments.

For western blotting, cells were harvested and lysed in urea-lysis buffer (8 M urea, 100 mM NaH₂PO₄, 10 mM Tris, pH 8.0) 72 h after reverse transfection of the siRNA. Shear force was applied using a 23-gauge syringe, and the cell debris were removed by centrifugation at 30.000 g for 15 min at 4° C. Then a Bradford assay was used to normalize total protein amounts, and appropriate amounts of protein lysate in 1× Laemmli-buffer were loaded onto an SDS-PAGE (4% stacking, 12% separating gel). Proteins were transferred on a PVDF membrane using a tank-blotting-system at 30 V overnight. The membrane was blocked in 5% nonfat dry milk TBST+50 μg/ml avidin for 2h at room temperature, and was afterwards incubated with the primary antibodies (5% nonfat dry milk TBST+1:1000 α-ADAR1, Santa Cruz, sc-73408 or α-ADAR2, Santa Cruz, sc-73409+1:40.000 α-beta-actin, Sigma Aldrich, A5441) at 4° C. overnight. The secondary antibodies (5% nonfat dry milk TBST+1:10.000 α-Mouse-HRP+1:50.000 Precision Protein™ StrepTactin-HRP Conjugate, Bio-Rad, #1610381) were incubated for 1.5h at room-temperature. After each antibody incubation, the membrane was washed 3×5 min with TBST. Detection was performed using 1 ml of Clarity Western ECL Substrate (Biorad) and a Fusion SL Vilber Lourmat (Vilber).

In Western Blot, only ADAR1p110 was found to be well expressed, whereas ADAR1p150 was only faintly visible but clearly inducible by IFN-α (FIG. 2D). ADAR2 was not detectable (data not shown). RNA interference was applied in order to knockdown specific ADAR isoforms. To this end, HeLa cells were reverse transfected in 12-well format with 2.5 pmol siRNA against ADAR1 (both isoforms, Dharmacon, SMARTpool: ON-TARGETplus ADAR (103) siRNA, L-008630-00-0005), ADAR1p150 (Ambion (Life Technologies), Sense strand: 5′-GCCUCGCGGGCGCAAUGAAtt (SEQ ID NO: 90); Antisense strand: 5′-UUCAUUGCGCCCGCGAGGCat (SEQ ID NO: 91)), ADAR2 (Dharmacon, SMARTpool: ON-TARGETplus ADARB1 (104) siRNA, L-009263-01-0005) or mock (Dharmacon, siGENOME Non-Targeting siRNA Pool #2, D-001206-14-05). 200 μl of transfection mix, containing 2.5 μl of the respective siRNA (1 nM) and 3 μl HiPerFect (Qiagen, Germany) and OptiMEM, were distributed evenly in each well before adding 800 μl DMEM+10% FBS containing 1.2×10⁵ HeLa cells. Medium was changed every 24 h. For RNA editing experiments, cells were detached 48 hours after siRNA transfection and were reverse-transfected with the respective ASO as described above.

When transfecting an siRNA against ADAR2 or mock, respectively, the editing yield remained unaffected at 35% and 70%, depending on IFN-α, respectively (FIG. 2C). However, the specific knockdown of the long isoform ADAR1p150 resulted in a decrease of the editing yield down to 10% and 20%. The concurrent knockdown of both ADAR1 isoforms abolished editing below detection. This suggests that both ADAR1 isoforms contribute to editing, however, the much weaker expressed p150 isoform of ADAR1 contributed most to the editing yields achieved. This is in good agreement with the observed positive effect of IFN-α treatment (FIG. 2), but also with the better performance of the ASO in ADAR1p150-expressing 293 Flp-In cells (FIG. 1C).

When varying the amount of ASO v9.5 between 20 pmol and 40 fmol/96 well (FIG. 2E), a sigmoidal dependency of the editing yield was observed, reaching the half-maximum yield at a dose of 0.2 pmol ASO/96 well (with IFN-α) and 0.4 pmol/well (without IFN). The maximum editing yield was obtained at ≥2 pmol/96 well. The potency in HeLa cells seems to be in a range similar to that for the transfection of siRNA duplexes for RNA interference.

The time profile of the editing yield was further assayed over five days after transfection of 5 pmol/well into quickly dividing HeLa cells (10% FBS). For that purpose, HeLa cells were transfected as described above. Prior to transfection, cells were treated with IFN-α for 24 hours (where indicated). Cells were harvested for RNA isolation at the respective time points indicated. For time points later than 24 hours post transfection, cells were detached after 24 hours and transferred into 24-well plates in order to avoid overgrowth of the cells. Medium (containing IFN-α where indicated) was changed every 24 hours. The maximum editing yield was typically observed in a time window of 12-48 hours after transfection and dropped down slowly (FIG. 2F).

In order to assess the scope of cell lines, in which the recruitment of endogenous ADAR works efficiently, ASO v9.5 was applied to a panel of 10 immortalized human standard (cancer) cell lines (FIG. 2G). All cells were cultured in DMEM+10% FBS+P/S. 5×10⁴ cells/96 well of the respective cell line [HeLa cells (Cat. No.: ATCC CCL-2), U2OS-Flp-In T-REx (kind donation from Prof. Elmar Schiebel), SK-N-BE(2) (Cat. No.: ATCC CRL-2271), SK-N-BE(2) (Cat. No.: ATCC CRL-2271), U87MG (Cat. No.: ATCC HTB-14), Huh7 (CLS GmbH, Heidelberg, Cat. No.: 300156), HepG2 (DSMZ, Braunschweig, Germany Cat. No.: ACC180), AKN-1 (kind donation from the Nüssler lab), empty HEK-Flp-ln T-REx (R78007, Thermo Fisher scientific, stably transfected with empty pcDNA5 vector) and A549 (European Collection of Authenticated Cell Cultures ECACC 86012804)] were reverse transfected with the respective ASO as described above for HeLa cells without further optimization. Only SH-SY5Y (Cat. No.: ATCC CRL-2266) cells were reverse transfected differently, in a 24-well format: to 100 μL transfection mix consisting of 2.5 μL Lipofectamine2000 and 25 pmol ASO in OptiMEM, 5×10⁵ cells in 500 μL medium (+3000 U IFN-α) were added. In some cell lines, like e.g. A549 and Huh7, the editing yield was comparable to HeLa cells, while others showed a lower editing yield. The lowest level of editing was obtained with the “empty” 293 Flp-ln cell line (empty pcDNA5 was integrated) with <11% yield under all conditions. Prior to IFN-α treatment, editing yields of 4%-34% (average 18.5%) were achieved. Similar as described before, the yields were 2-3fold higher after IFN-α treatment ranging from 11%-73% (average 46.8%).

In order to better assess the potential therapeutic scope of ADAR-recruiting ASOs, a panel of seven primary cells from different tissues was tested, including fibroblasts (from a Parkinson patient), and commercially acquired astrocytes, hepatocytes (several donors), epithelial cells from the retina and the bronchia, and endothelial cells from arterial and venous vessels (FIG. 2H). All primary cells were purchased from Lonza except for the primary fibroblasts, which were a kind gift from the Valente lab. Primary fibroblasts were cultured in DMEM+20% FBS. The other cell lines were cultured in their respective commercial media as indicated: Human Umbilical Vein Endothelial Cells (HUVEC, Lonza Cat. No.: CC-2517) and Human Aortic Endothelial Cells (HAEC, Lonza Cat. No.: CC-2535) in medium 200PRF (Thermo Fisher Scientific Cat. No.: M200PRF500) with Low Serum Growth Supplement (LSGS Thermo Fisher Scientific Cat. No.: S00310), Normal Human Astrocytes (NHA, Lonza Cat. No.: CC-2565) in ABM Basal Medium (Lonza Cat. No.: CC-3187) with AGM SingleQuot Kit Suppl. & Growth Factors (Lonza Cat. No.: CC-4123), Human Retinal Pigment Epithelial Cells (H-RPE, Lonza Cat. No.: 194987) in EpiLife Medium (Thermo Fisher Scientific Cat. No.: MEPI500CA) with Human Corneal Growth Supplement (Thermo Fisher Scientific Cat. No.: S0095), Normal Human Bronchial Epithelial Cells (NHBE, Lonza Cat. No.: CC-2540) in Airway Epithelial Cell Basal Medium (LGC Standard Cat. No.: ATCC-PCS-300-030) with Bronchial Epithelial Cell Growth Kit (LGC Standard Cat. No.: ATCC-PCS-300-040) and Primary Human Hepatocytes (PHH, Lonza Cat. No.: HUCPI) were thawed in Cryo HH thawing media (Lonza Cat. No.: MCHT50), seeded in Hepatocyte Plating Medium w/Supplement (Lonza Cat. No.: MP100) and 6 hours after seeding cultured in Hepatocyte Maintenance Media w/Supplement (Lonza Cat. No.: MM250). 3.5×10⁴ HUVEC and HAEC, 1×10⁵ NHA, H-RPE and NHBE and 4.5×10⁵ PHH were seeded 24 hours before ASO transfection in 24-well format. For PHH rat collagen I-coated 24-well plates (GreinerBioOne) were used. Shortly before transfection, medium was changed (plus 3000U IFN-α in 500 μL medium/well if indicated). For each well, 1.5 μL Lipofectamine RNAiMAX (Thermo Fisher Scientific) and 25 pmol ASO were diluted separately in a total volume of 50 μL OptiMEM, respectively. After 5 minutes of incubation, the two solutions were combined and after another 20 min of incubation, the 100 μL transfection mix was equally distributed in one well. After 24h cells were harvested for RNA isolation and sequencing. Unexpectedly, higher editing levels were detected in primary cells compared to immortalized cells, obtaining editing levels of 10%-63% (average 31.5%). Notably, in both primary hepatocyte samples and in the patient fibroblast, the editing levels were higher than in HeLa cells. Again, editing yields increased in all cells after IFN-α treatment yielding a range from 35%-77% (average 62.6%). A series of ASO dilutions (25-0.2 pmol ASO v9.5/24 well, no IFN treatment) was transfected into hepatocytes of donor #1 and #2, demonstrating a clear close-dependency (FIG. 2H).

### Example 3

Following the characterization of ASO design 9.4 for the editing of 5′-UAG triplets in the 3′-UTR, the editing of a 5′-UAG triplet in the ORF of GAPDH in ADAR-expressing 293 cell lines was tested with an ASO based on v9.4 (see also Example 1). Comparison of the editing yields obtained with the three ADARs showed that the editing yields in the ORF followed the same trend as in the 3′-UTR before (ADAR1p150>ADAR1p110 ADAR2), albeit with generally lower editing yields (11%-55%, see FIG. 3A).

The ASO architecture was further optimized in order to improve the on-target binding kinetics by increasing the length of specificity domain and by including LNA modifications. We identified ASO design v25, which comprises of the unaltered ADAR-recruiting domain, but contained a 40 nt specificity domain, which was partly modified by 2′-O-methylation, phosphorothioate linkage and contained three LNA modifications (FIG. 3B). After transfection into HeLa cells, ASO v25 achieved editing yields of 26±3% without IFN- and of 42.7±1.5% with IFN-. Notably, the chemical modification of the ADAR-recruiting domain was important. Without chemical modification, v25 gave no editing in absence of IFN- and only moderate editing with IFN-(13.7±3.5%, FIG. 4). The new design v25 was also tested in several primary cells for the editing of the 5′-UAG site in the ORF of GAPDH. Prior to IFN-treatment, editing levels of 12.7±2.1% (fibroblast), 9.3±0.6% (RPE), and 38% (hepatocyte, one donor) were obtained. As before, IFN-treatment improved the editing levels to 22.7±0.6% (fibroblast), 32.3±4.5% (RPE), and 45% (hepatocyte, one donor).

### Example 4

In order to evaluate the therapeutic potential of such ASOs, the editing of two therapeutically relevant deamination sites was tested. First, the phosphorylation site in endogenous STAT1 (Tyr701) was targeted, deamination of which switches function of the protein as a transcription factor. After editing, the respective 5′-UIU codon encodes for Cys, an amino acid that is unable to mimic phosphorylated Tyr. An ASO based on the v25 design described above was used in these experiments. Editing yields of 21.0±6.2% were achieved in primary fibroblasts and up to 7% in RPE cells prior to IFN-α treatment (FIG. 3D). In presence of IFN-α, the yields increased to 32±7% (fibroblasts) and 19.7±2.5% (RPE). Similar values were obtained in HeLa cells. Overall, editing of the endogenous STAT1 transcript by recruiting endogenous ADAR was possible in moderate yields in primary cell lines.

As a second site, the editing of the PiZZ mutation (E342K) in the SERPINA1 transcript, the most common cause of α1-antitrypsin deficiency (A1AD), was tested. Loss of antitrypsin, which regulates neutrophil elastase activity, causes severe damage of the lungs. Furthermore, mutated antitrypsin accumulates in the liver and leads to severe liver damage. First, the editing of the E342K mutation (5′-CAA triplet) was tested upon overexpression of the mutated SERPINA1 cDNA in ADAR1p150-expressing 293 cells applying an ASO build on the v9.4 design. In order to obtain SERPINA1 cDNA for cloning, total RNA was isolated from HepG2 cells and reverse transcribed. The E342K mutation was inserted into the cDNA by PCR and both SERPINA1 wild-type and the E342K mutant were each cloned on a pcDNA3.1 vector under control of the CMV promotor using HindIII and ApaI restriction. For genomic integration of SERPINA1 using the piggyBac transposon system, the wild-type and mutant cDNA was cloned on a PB-CA vector using the same restriction sites as above. 1×10⁶ HeLa cells were seeded in a six-well plate 24 hours before transfection. 1 μg of the piggyBac transposase vector (Transposagen Biopharmaceuticals) and 2.5 μg of the SERPINA1 PB-CA vector were co-transfected using 10.5 μL FuGENE6 (Promega) according to the manufacturer's protocol. After 24 hours, cells were selected for 2 weeks in DMEM+10% FBS medium containing 10 μg/mL puromycin. For editing, stably transfected or plasmid transfected (300 ng plasmid/0.9 μL FuGENE6 for Hela and 100 ng plasmid/0.3 μL Lipofectamine2000 for Flp-ADAR1p150 cells) cells were reverse transfected with the respective ASO as described above. After 24 hours, cell culture supernatant was collected for the A1AT-ELISA and cells were harvested for RNA isolation and sequencing. The A1AT-ELISA was performed with a commercial kit (cat. no.: ab108799, Abcam) according to the manufacturer's protocol. Samples from three biological replicates were measured in technical duplicates. The MAT protein amount was calculated from a standard curve using linear regression.

Only in presence of the ASO, an editing yield of 29±2% was determined at the targeted site (FIG. 3E). The secretion of α1-antitrypsin (A1AT) was measured by an ELISA assay and normalized to the secretion of cells transfected with wildtype SERPINA cDNA. The secretion level was elevated from 14±1.8% prior to 27±4.3% after repair. Other than the 5′-UAG triplet, the 5′-CAA triplet contains an editable adenosine in closest proximity to the targeted A. Indeed, some minor editing was detected at the proximal site, a problem that may be solved by further chemical modification of the ASO around the target nucleotide. To test the repair of A1AD-causing mutation with endogenous ADAR, a HeLa cell line stably expressing mutated SERPINA1 was created using the piggyBac system or by plasmid-borne overexpression of SERPINA1 cDNA. By applying an ASO based on the v25 design, editing levels of 19±2% (integrated cDNA, with IFN-α) and 21±4% (transient expression of cDNA, with IFN-α) were obtained by recruitment of endogenous ADAR.

### Example 5

In order to test the guideRNA stabilities, guideRNAs have been incubated for a defined amount of time (0 min, 5 min, 10 min, 1 h, 3 h, 6 h, 12 h or 24 h) in PBS buffer containing 10% FBS. After incubation, the guideRNAs were separated on a 15% Urea (7M)-PAGE, stained with SYBR Gold and were photographed and quantified with a Typhoon FLA biomolecular imager. The guideRNAs with the unmodified 3 nt anticodon typically had very short half-lifes in serum (minutes). The guideRNA with a 3′-UCU anticodon targeting the 5′-AAA codon, e.g. FIG. 6A), was essentially undetectable at the first incubation time point (5 min) due to degradation. However, already single backbone modification with 2′-F or 2′-O-methyl gave improved stability (FIGS. 6A and B). For the anticodon 3′-ACC, it was shown, for example, that the half-life was strongly improved from below 5 min to around 24 h (ca. 300 fold improvement) by several modification patterns that each modify all nucleotides in the anticodon. Further, the editing yield was also increased by these modifications compared to the guideRNA (BG-85) unmodified in the 3 nt anticodon (see, for example, BG-150/BG-151, FIG. 6C).

### Example 6

In a parallel approach, guideRNAs conjugated with a coupling agent were employed for editing endogenous transcripts with tagged ADARs. For example, BG-conjugated guideRNAs were used in combination with SNAP-tagged ADARs (see FIG. 7). BG-conjugated gRNAs were synthesized and PAGE-purified from commercially acquired oligonucleotides containing a 5′-amino-C6 linker (BioSpring, Germany) as described by Hanswillemenke et al. (J. Am. Chem. Soc. 2015, 137, 15875-15881). The sequences and chemical modification of all guideRNAs are provided in Table 2.

TABLE 2
guideRNAs for use with tagged ADARs

| | |
|---|---|
| target | gRNA sequence |

editing of various endogenous transcripts:

| | |
|---|---|
| 5′-UTR | 5′-UsCsAUUAAACC CCA GAGUCsCsGsGsA-3′ |
| SNAP-ADAR | (SEQ ID NO: 92) |
| 5′-UTR | 5′-UsCsUGAAUAAU CCA GGAAAsAsGsCsA-3′ |
| GAPDH | (SEQ ID NO: 93) |
| isoform | |
| 2 | |
| ORF | 5′-UsAsUAGGGGUG CCA AGCACsUsUsGsG-3′ |
| #1 | (SEQ ID |
| GAPDH | NO: 94) |
| ORF | 5′-UsAsUGGUUUUU CCA CACGGsCsAsGsG-3′ |
| #2 | (SEQ ID |
| GAPDH^{b)} | NO: 95) |
| ORF | 5′-GsGsUGCAGAUU CCA GGUGGsGsAsCsG-3′ |
| #1 | (SEQ ID |
| GUSB | NO: 96) |
| ORF | 5′-AsCsAGACUUGG CCA CUGAGsGsGsGsG-3′ |
| #2 | (SEQ ID |
| GUSB | NO: 97) |
| 3′-UTR | 5′-UsAsUGUGUCGG CCA CGGAAsCsAsGsG-3′ |
| SNAP- | (SEQ ID |
| ADAR | NO: 98) |
| 3′-UTR | 5′-AsAsUAAGGGGU CCA CAUGGsCsAsAsC-3′ |
| GAPDH^{C)} | (SEQ ID NO: 99) |
| 3′-UTR | 5′-UsCsGAGCAAUG CCA UCACCsUsCsCsC-3′ |
| ACTB | (SEQ ID NO: 100) |
| 3′-UTR | 5′-UsAsUUUCCCUG CCA GAAUAsGsAsUsG-3′ |
| GUSB | (SEQ ID NO: 101) |
| KRAS | 5′-GsAsUGCUCCAA CCA CCACAsAsGsUsU-3′ |
| target | (SEQ ID NO: 102) |
| A/1 | |
| KRAS | 5′-CsCsUCUCUUGC CCA CGCCAsCsCsAsG-3′ |
| target | (SEQ ID NO: 103) |
| 2 | |
| STAT1 | 5′-CsUsCUCUUGAU ACA UCCAGsUsUsCsC-3′ |
| Y701 | (SEQ ID NO: 104) |

editing of all 16 adenosine-
containing triplets in GAPDH isoform 1:

| | |
|---|---|
| 5′-GAA | 5′-CsAsCAUGCGAU UCC CAUUGsAsUsGsA-3′ |
| | (SEQ ID NO: 105) |
| 5′-GAU | 5′ - UsAsUCGACCAA ACC CGUUGsAsCsUsC-3′ |
| | (SEQ ID NO: 106) |
| 5′-GAC | 5′-CsAsCGUCAUGA GCC CUUCCsAsCsCsA-3′ |
| | (SEQ ID NO: 107) |
| 5′-GAG | 5′- AsAsCGAGGGAU CCC GCUCCsUsGsGsA-3′ |
| | (SEQ ID NO: 108) |
| 5′-CAA | 5′-GsAsAGAGGCUG UCG UCAUAsCsUsUsC-3′ |
| | (SEQ ID |
| | NO: 109) |
| 5′-CAU | 5′-CsAsACACGUCA ACG AAGGGsGsUsCsA-3′ |
| | (SEQ ID NO: 110) |
| 5′-CAC | 5′-AsAsCGCCAGGG GCG CUAAGsCsAsGsU-3′ |
| | (SEQ ID NO: 111) |
| 5′-CAG | 5′-UsAsCGCAUCGA CCG UCCUCsAsUsCsA-3′ |
| | (SEQ ID |
| | NO: 112) |
| 5′-AAA | 5′-UsAsCAUGACCC UCU UGGCUsCsCsCsC-3′ |
| | (SEQ ID NO: 113) |
| 5′-AAU | 5′-GsAsCUACCCAA ACU CGUUGsUsCsAsU-3′ |
| | (SEQ ID NO: 114) |
| 5′-AAC | 5′-AsGsUCGCCACA GCU UCCCGsGsAsGsG-3′ |
| | (SEQ ID NO: 115) |
| 5′-AAG | 5′-UsCsUAUAUCCA CCU UACCAsGsAsGsU-3′ |
| | (SEQ ID NO: 116) |
| 5′-UAA | 5′-AsGsGAGGGGUC UCA CUCCUsUsGsGsA-3′ |
| | (SEQ ID |
| | NO: 117) |
| 5′-UAU | 5′-CsUsACGCAACA ACA UCCACsUsUsUsA-3′ |
| | (SEQ ID NO: 118) |
| 5′-UAC | 5′-CsCsGAGCGCCA GCA GAGGCsAsGsGsG-3′ |
| | (SEQ ID NO: 119) |
| 5′-UAG | 5′-UsAsUGGUUUUU CCA GACGCsCsAsCsG-3′ |
| | (SEQ ID |
| | NO: 120) |

avoiding off-target editing
of neighbouring adenosine:

| | |
|---|---|
| 5′-CAA | 5′-GsAsAGAGGCUCU CG UCAUAsCsUsUsC-3′ |
| methoxy | (SEQ ID |
| | NO: 121) |
| 5′-CAA | 5′-GsAsAGAGGCUG [symbol] CG UCAUAsCsUsUsC-3′ |
| fluoro | (SEQ ID |
| | NO: 122) |
| 5′-AAA | 5′-UsAsCAUGACCCU CU UGGCUsCsCsCsC-3′ |
| methoxy | (SEQ ID NO: 123) |
| 5′-AAA | 5′-UsAsCAUGACC [symbol] CU UGGCUsCsCsCsC-3′ |
| fluoro | (SEQ ID NO: 124) |
| 5′-AAC | 5′-AsGsUCGCCACA GC UUCCCGsCsAsGsG-3′ |
| methoxy | (SEQ ID NO: 125) |
| 5′-AAC | 5′-AsGsUCGCCACA GC [symbol] UCCCGsCsAsGsG-3′ |
| fluoro | (SEQ ID NO: 126) |
| 5′-UAA | 5′-AsGsGAGGGGUCU CA CUCCUsUsGsGsA-3′ |
| methoxy | (SEQ ID |
| | NO: 127) |
| 5′-UAA | 5′-AsGsGAGCGCUC [symbol] CA CUCCUsUsGsGsA-3′ |
| fluoro | (SEQ ID |
| | NO: 128) |

Legend to Table 2:
Nucleotides highlighted in bold are unmodified and are placed opposite the triplet with the target adenosine in the middle.
Nucleotides highlighted in italic are modified with 2′-O-methylation, 2′-fluorinated nucleotides are grayed out.
The backbone contains terminal phosphorothioate linkages as indicated by “s”.
The first three nucleotides at the 5′-end are not complementary to the mRNA substrate, but serve as linker sequence between gRNA and SNAP-tag.

For this study, all NH₂-guideRNAs were purchased from Biospring (Germany) as HPLC-purified ssRNAs with a 5′-C6 amino linker. As an alternative to commercial BG derivatives, our protocol can be used to introduce the BG moiety. Benzylguanine connected to a carboxylic acid linker2,3 (12 μl, 60 mM in DMSO) was in-situ activated as an OSu-ester by incubation with EDCl.HCl (12 μl, 17.4 mg/ml in DMSO) and NHS (12 μl, 17.8 mg/ml in DMSO) for 1 h at 30° C. Then, the NH₂-guideRNA (25 μl, 6 μg/μl) and DIPEA (12 μl, 1:20 in DMSO) were added to the pre-activation mix and incubated (90 min, 30° C.).20 19 The crude BG-guideRNA was purified from unreacted NH₂-guideRNA by 20% urea PAGE and then extracted with H₂O (700 μl, overnight at 4° C.). RNA precipitation was done with sodium acetate (0.1 volumes, 3.0 M) and ethanol (3 volumes, 100%, overnight at −80° C.). The BG-guideRNA was washed with ethanol (75%) and dissolved in water (60 μl).

Cell lines were generated that stably express SNAP-ADAR1 (SA1), SNAP-ADAR2 (SA2), 2 and their hyper-active E Q variants 10 SA1Q and SA2Q. Each respective enzyme (SA1 (wt & Q) and SA2 (wt and Q)) was integrated as a single copy under control of the dox-inducible CMV promotor at the FRT site into the genome of 293 Flip-In cells (R78007, Thermo Fisher scientific) as described before (see Wettengel, J., Reautschnig, J., Geisler, S., Kahle, P. J., Stafforst, T. Harnessing human ADAR2 for RNA repair—Recoding a PINK1 mutation rescues mitophagy. Nucl. Acids Res. 45, 2797-2808 (2017); or Cox, D. B. T., Gootenberg, J. S., Abudayyeh, O. O., Franklin, B., Kellner, M. J., Joung, J., Zhang, F. RNA editing with CRISPR-Cas13, Science, 10.1126/science.aaq0180 (2017). Enzyme expression of all four enzymes was inducible by doxycycline (10 ng/ml) to roughly comparable levels as validated by Western blot and fluorescence microscopy (data not shown). Also at the RNA level, the expression levels of SA1 (wt & Q) and SA2 (wt and Q) were roughly comparable with average FPKM values of 679 and 814 for SA1(Q) and SA2(Q), respectively. The E Q mutation did not change the protein localization. SA1(Q) is localized to cytoplasm and nucleoplasm; SA2(Q) is mainly localized to cytoplasm. In order to determine the location of the different SNAP-ADAR proteins, 1×10⁵ cells were seeded in 500 μl selection media with or without doxycline (10 ng/ml) on poly-D-lysine-coated cover slips in a 24-well format. After one day, BG-FITC labeling of the SNAP-tag and nuclear staining was done. To validate SNAP-ADAR protein amounts, Western blot analysis was used. For this, 3×10⁵ cells were seeded in 500 μl selection media with or without doxycline (10 ng/ml) in a 24-well format for one day. Then, cells were lysed with urea buffer (8 M urea in 10 mM Tris, 100 mM NaH₂PO₄, pH 8.0). Protein lysate (5 μg) was separated by SDS-PAGE and transferred onto a PVDF membrane (Bio-Rad Laboratories, USA) for immunoblotting with primary antibodies against the SNAP-tag (1:1000, P9310S, New England Biolabs, USA) and g-actin (1:40000, A5441, Sigma Aldrich, USA). Afterwards, the blot was incubated with HRP-conjugated secondary antibodies against rabbit (1:10000, 111-035-003, Jackson Immuno Research Laboratories, USA) and mouse (1:10000, 115-035-003, Jackson Immuno Research Laboratories, USA) and visualized by enhanced chemiluminescence.

Editing was initiated by transfection of the short, chemically stabilized BG-guideRNA, and was analyzed for formal A-to-G conversion in cDNA at specific 5′-UAG triplets in the 3′-UTRs of the four targeted endogenous mRNAs: ACTB, GAPDH, GUSB, and SA1/2. For both wildtype enzymes (SA1/2), editing yields of 40-80% were achieved (FIG. 8a) depending on the target. Applying the hyperactive mutants (SA1Q/SA2Q) raised the yields to 65-90%. The maximum editing yield (80-90%) was obtained almost 3h post transfection (FIG. 1b), stayed constant for 3 days, and then declined slowly, probably due to dilution of the guideRNA-enzyme conjugate by cell division. The activated enzymes (SA1Q&SA2Q) were up to 12fold more potent compared to the wildtype enzymes (SA1&SA2), achieving the half-maximum editing yield already with 0.15 pmol/well compared to 1-2 pmol/well (FIG. 1c). Concurrent editing of all four transcripts was tested by cotransfection of four guideRNAs. Notably, the yields remained unchanged (FIG. 1a). Editing yields were higher in the 3′-UTR compared to ORF and 5′-UTR (FIG. 1d), probably due to interference with translation. The faster enzymes (SA1Q & SA2Q) boosted the yields in the 5′-UTR from 25-50% to 60-75% and in the ORF from 15-60% to 50-85% (FIG. 1d). Furthermore, translation inhibition with puromycin increased ORF editing in SA1/2 cells to the level of 3′-UTR editing (data not shown). To assess the codon scope, all 16 conceivable 5′-NAN triplets in the ORF of endogenous GAPDH were tested for SA1Q and SA2Q. Yields ranging from very little to almost quantitative were obtained, reflecting the known preferences of ADARs (FIG. 1e). While editing was generally difficult for 5′-GAN triplets (<30%), significant yields (>50%) were achieved for 10/16 triplets. For 7/16 triplets, excellent editing yields (>70%) were obtained for at least one enzyme.

### Example 7

A major objective in RNA editing is the suppression of off-site editing (see FIG. 9a). It was therefore tested, whether off-site editing can be avoided by using chemically modified versions of the guideRNAs described herein. Only for adenosine-rich triplets (AAC, AAA, UAA, CAA) some off-target editing was detected, mainly with SA2Q (5-75%) and mainly for the CAA triplet (FIG. 9b, right diagram, “r”). Off-target editing was higher if three natural nucleotides were present in the guideRNA opposite the targeted adenosine (FIG. 9b, in particular right diagram, “r”). Careful inclusion of further chemical modifications (2′-methoxy, 2′-fluoro) restricted off-target editing at the CAA triplet down to 20%, and limited off-target editing at all other sites to <10% without reducing on-target editing (FIG. 9b, “M”, “F”). Notably, at least for AAA the additional modification even elevated the on-target yield from 40% to 50%.

### Example 8

Branched linkers and multiple copies of the BG-derived recruiting moieties were tested with regard to their effect on RNA editing. To this end, various guideRNAs were tested side-by-side against the Tyr701 codon in the endogenous STAT1 transcript in 293-Flp-In cells expressing SNAP-ADAR1Q (24 h induction with 10 ng/ml doxycycline prior to guideRNA transfection, editing analysis was done 24 h post guideRNA transfection). Specifically, guideRNAs were applied that contained either a 5′-amino linker or both, a 5′- and a 3′-amino linker and coupled to one or two of the recruiting moieties, respectively. The resulting guideRNAs can potentially recruit from one to eight SNAP-ADAR1Q deaminases, as illustrated by FIG. 11. In the presence of saturating amounts of guideRNA (1 pmol/well or above) almost all of the guideRNAs achieved the same editing yields (70-80%). Only the single 1×BG guideRNAs did not achieve the maximum yield but stopped at a yield of ca. 60%. The guideRNAs that allow for recruiting more than one SNAP-ADAR1Q showed improved potency, indicating that they maintained a high editing yield, when the amount of guideRNA was reduced. For instance, in the case of single 1×BG, the editing yield dropped to 22% and below detection when reducing the guideRNA amount to 0.1 pmol/well and 0.01 pmol/well. In contrast, for the double 2×BG and for the double 4×BG guideRNAs, the editing yields remained at 58% and 65%, respectively, with 0.1 pmol/well and dropped to only 17% and 10% with 0.01 pmol/well, thus clearly improving the potency of the guideRNA.

1. Artificial nucleic acid for site-directed editing of a target RNA, the artificial nucleic acid comprising
a) a targeting sequence, which comprises a nucleic acid sequence complementary to a target sequence in the target RNA,
and
b) a recruiting moiety for recruiting a deaminase,
wherein the targeting sequence comprises at least one nucleotide, wherein the nucleobase is chemically modified, and/or
wherein the targeting sequence comprises at least one backbone modification.

2. The artificial nucleic acid according to claim 1, wherein the targeting sequence comprises at least one chemically modified nucleotide, which is chemically modified at the 2′ position.

3. The artificial nucleic acid according to claim 2, wherein
the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro; and/or
wherein the chemically modified nucleotide is selected from the group consisting of a locked nucleic acid (LNA) nucleotide, an ethylene bridged nucleic acid (ENA) nucleotide and an (S)-constrained ethyl cEt nucleotide.

4. The artificial nucleic acid according to any of the preceding claims, wherein the targeting sequence comprises at least one backbone modification and wherein a nucleotide comprises a modified phosphate group, preferably selected from the group consisting of a phosphorothioate, a phosphoroselenate, a borano phosphate, a borano phosphate ester, a hydrogen phosphonate, a phosphoroamidate, an alkyl phosphonate, an aryl phosphonate and a phosphotriester.

5. The artificial nucleic acid according to any of the preceding claims, wherein at least 40% of the nucleotides of the targeting sequence are chemically modified at the 2′ position.

6. The artificial nucleic acid according to any of the preceding claims, wherein the targeting sequence comprises at the position corresponding to a nucleotide to be edited, preferably an adenosine or a cytidine nucleotide to be edited, in the target sequence a cytidine nucleotide or a variant thereof, a deoxycytidine nucleotide or a variant thereof, or an abasic site.

7. The artificial nucleic acid according to any of the preceding claims,
wherein at least one, preferably both, of the two nucleotides or variants thereof, which are positioned 5′ or 3′ of the position corresponding to a nucleotide to be edited in the target sequence, is chemically modified at the 2′ carbon atom, wherein the 2′ carbon atom is linked to a substituent selected from the group consisting of a halogen, an alkoxy group, a hydrogen, an aryloxy group, an amino group and an aminoalkoxy group, preferably selected from 2′-O-methyl, 2′-O-methoxyethyl, 2′-hydrogen and 2′-fluoro;
and/or
wherein at least one, preferably both, of the two nucleotides or variants thereof, which are positioned 5′ or 3′ of the position corresponding to a nucleotide to be edited in the target sequence, comprises a modified phosphate group, preferably a phosphorothioate group.

8. The artificial nucleic acid according to any of the preceding claims, wherein the targeting sequence comprises at the position corresponding to a nucleotide to be edited in the target sequence a cytidine nucleotide or a variant thereof, a deoxycytidine nucleotide or a variant thereof, or an abasic site, and
wherein the nucleotide, which is positioned 5′ of the position corresponding to the nucleotide to be edited, is a pyrimidine nucleotide, preferably a pyrimidine ribonucleotide or a pyrimidine deoxynucleotide, and wherein said pyrimidine nucleotide comprises a nucleobase, which is chemically modified at the 2′ position, preferably by 2′-hydrogen, 2′-O-methyl, 2′-O-methoxyethyl or 2′-O-fluoro.

9. The artificial nucleic acid according to any of the preceding claims,
wherein the targeting sequence comprises the nucleic acid sequence
3′AcC5′,
wherein
A is an adenosine nucleotide or a variant thereof, preferably an adenosine ribonucleotide or a deoxyadenosine nucleotide;
c is a cytidine nucleotide or a variant thereof, a deoxycytidine nucleotide or a variant thereof, or an abasic site, at the position corresponding to a nucleotide, preferably an adenosine or a cytidine, more preferably an adenosine, to be edited in the target sequence; and
C is a cytidine nucleotide or a variant thereof, preferably a cytidine ribonucleotide, a modified cytidine ribonucleotide, a deoxycytidine nucleotide or a modified deoxycytidine nucleotide, more preferably a deoxycytidine nucleotide or a modified deoxycytidine nucleotide.

10. The artificial nucleic acid according to any of the preceding claims,
wherein the targeting sequence comprises the nucleic acid sequence
3′As*cC*5′,
wherein
As is an adenosine nucleotide or a variant thereof, preferably an adenosine ribonucleotide or a deoxyadenosine nucleotide, further comprising a phosphorothioate group;
c is a cytidine nucleotide or a variant thereof, a deoxycytidine nucleotide or a variant thereof, or an abasic site, at the position corresponding to a nucleotide, preferably an adenosine or a cytidine, more preferably an adenosine, to be edited in the target sequence; and
C is a cytidine nucleotide or a variant thereof;
wherein an asterisk (*) indicates a chemical modification of the preceding nucleotide at the 2′ carbon atom with 2′-hydrogen (2′-deoxy), 2′-O-methyl, 2′-O-methoxyethyl or 2′-fluoro.

11. The artificial nucleic acid according to any of claims 1 to 8,
wherein the targeting sequence comprises the nucleic acid sequence
3′Us*cC*5′,
wherein
Us is an uridine nucleotide or a variant thereof, preferably an uridine ribonucleotide or a deoxyuridine nucleotide, further comprising a phosphorothioate group;
c is a cytidine nucleotide or a variant thereof, a deoxycytidine nucleotide or a variant thereof, or an abasic site, at the position corresponding to a nucleotide, preferably an adenosine or a cytidine, more preferably an adenosine, to be edited in the target sequence; and
C is a cytidine nucleotide or a variant thereof;
wherein an asterisk (*) indicates a chemical modification of the preceding nucleotide at the 2′ carbon atom with 2′-hydrogen (2′-deoxy), 2′-O-methyl, 2′-O-methoxyethyl or 2′-fluoro;

12. The artificial nucleic acid according to any of the preceding claims, wherein at least two of the five nucleotides at the 3′ terminus of the targeting sequence comprise a modified phosphate group, preferably a phosphorothioate group.

13. The artificial nucleic acid according to any of the preceding claims, wherein at least two of the five nucleotides at the 3′ terminus of the targeting sequence are LNA nucleotides, ENA nucleotides or (S)-constrained ethyl cEt nucleotides.

14. The artificial nucleic acid according to any of the preceding claims, wherein the targeting sequence comprises
at least one nucleotide comprising a modified phosphate group, preferably a phosphorothioate nucleotide;
at least one nucleotide selected from the group consisting of an LNA nucleotide, an ENA nucleotide and an (S)-constrained ethyl cEt nucleotide, preferably an LNA nucleotide; and
at least one nucleotide comprising a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro.

15. The artificial nucleic acid according to any one of the preceding claims, wherein the targeting sequence is characterized by a modification pattern according to any one of formulae (Ia), (Ib) or (Ic):
3′NₐCN_{b}5′ (Ia)
wherein
N is a nucleotide or a variant thereof, preferably a ribonucleotide or a variant thereof, a deoxynucleotide or a variant thereof, more preferably a modified ribonucleotide, or a modified deoxynucleotide;
C is the nucleotide at the position corresponding to the nucleotide to be edited in the target sequence and wherein C is a cytidine nucleotide or a variant thereof, a deoxycytidine nucleotide or a variant thereof, or an abasic site;
a is an integer in a range from 1 to 40, preferably from 6 to 10;
b is an integer in a range from 4 to 40; and
wherein a+b is in a range from 15 to 80;
3′N_{c}Ns_{d}NₐCN_{b}NsₑN_{f}5′ (Ib)
wherein
N is a nucleotide or a variant thereof, preferably a ribonucleotide or a variant thereof, a deoxynucleotide or a variant thereof, more preferably a modified ribonucleotide, or a modified deoxynucleotide;
C is the nucleotide at the position corresponding to the nucleotide to be edited in the target sequence and wherein C is a cytidine nucleotide or a variant thereof, a deoxycytidine nucleotide or a variant thereof, or an abasic site;
Ns is a nucleotide comprising a modified phosphate group, preferably a phosphorothioate group;
c is an integer in a range from 0 to 4;
d is an integer in a range from 1 to 10;
a is an integer in a range from 1 to 26;
b is an integer in a range from 4 to 40;
e is an integer in a range from 0 to 4;
f is an integer in a range from 0 to 4;
wherein a+d+c is in a range from 1 to 40;
wherein b+e+f is in a range from 4 to 40; and
wherein a+d+c+b+e+f is in a range from 15 to 80;
3′N_{c}NI_{g}NₕNIᵢNₐCN_{b}NIⱼNₖNIₗNₘ5′ (Ic)
wherein
N is a nucleotide or a variant thereof, preferably a ribonucleotide or a variant thereof, a deoxynucleotide or a variant thereof, more preferably a modified ribonucleotide, or a modified deoxynucleotide;
C is the nucleotide at the position corresponding to the nucleotide to be edited in the target sequence and wherein C is a cytidine nucleotide or a variant thereof, a deoxycytidine nucleotide or a variant thereof, or an abasic site;
NI is an LNA nucleotide or a modified LNA nucleotide;
c is an integer in a range from 0 to 4, preferably from 1 to 3;
g, i is an integer in a range from 1 to 5;
h is an integer in a range from 1 to 30, preferably from 1 to 5;
a is an integer in a range from 1 to 15;
b is an integer in a range from 4 to 30;
j is an integer in a range from 0 to 5, preferably from 1 to 3;
k is an integer in a range from 4 to 30;
l is an integer in a range from 0 to 5, preferably from 1 to 3;
m is an integer in a range from 0 to 3;
wherein c+g+h+i+a is in a range from 1 to 40;
wherein b+j+k+I+m is in a range from 4 to 40; and
wherein c+g+h+i+a+b+j+k+I+m is in a range from 15 to 80.

16. The artificial nucleic acid according to any of the preceding claims, wherein the targeting sequence is characterized by a modification pattern selected from any one of the formulae II(a) to II(l):
3′Ns₄N₆CN₇₋₂₉5′; (a)
3′Ns₄N₆₋₁₀CN₉₋₁₂Ns₂5′; (b)
3′Ns₂N₁₁₋₁₅CN₉₋₁₂Ns₂5′; (c)
3′NIs₂Ns₂NIN₆₋₁₀CN₅₋₉NI₂NNs₂5′; (d)
3′NIsNsNIsNsN₆₋₁₀CN₄₋₈NINNINNs₂5′; (e)
3′NsNIsNsNIsN₆₋₁₀CN₃₋₇NINNIN₂Ns₂5′; (f)
3′Ns₂NNINNIN₆₋₁₀CN₄₋₈NINNINNs₂5′, (g)
3′NsNIsNs₂NIN₅CN₅NIN₁₋₂₃5′; (h)
3′NIsNsNIsNsN₈CN₆NIN₁₋₂₃5′ (i)
3′NsNIsNs₂NIN₅CN₅NIN₂₀NI₂5′; (j)
3′NIsNsNIsNsN₈CN₆NIN₂₀NI₂5′; and (k)
3′Ns₄N₆CN₉Ns₂5′, (l)
wherein
N is a nucleotide or a variant thereof, preferably a ribonucleotide or a variant thereof, a deoxynucleotide or a variant thereof, more preferably a modified ribonucleotide, or a modified deoxynucleotide;
Ns is a nucleotide comprising a modified phosphate group, preferably a phosphorothioate group;
NI is an LNA nucleotide or a modified LNA nucleotide;
NIs is an LNA nucleotide or a modified LNA nucleotide, further comprising a modified phosphate group, preferably a phosphorothioate group;
C is the nucleotide at the position corresponding to the nucleotide to be edited in the target sequence and wherein C is a cytidine nucleotide or a variant thereof, a deoxycytidine or a variant thereof, preferably a deoxycytidine nucleotide, or an abasic site.

17. The artificial nucleic acid according to any of the preceding claims, wherein the targeting sequence comprises a nucleic acid sequence, wherein,
with the exception of the cytidine nucleotide or the variant thereof, the deoxycytidine nucleotide or the variant thereof, or the abasic site, at the position corresponding to the nucleotide to be edited in the target sequence,
with the exception of LNA nucleotides, and
optionally with the exception of at least one of the two nucleotides, which are positioned 5′ or 3′ to said nucleotide at the position corresponding to the nucleotide to be edited in the target sequence,
all nucleotides are chemically modified at the 2′ carbon atom, which is linked to a substituent selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro.

18. The artificial nucleic acid according to any of the preceding claims, wherein the targeting sequence comprises a nucleic acid sequence selected from the group consisting of
(SEQ ID NO: 1)
5′ U*U*C*A*C*U* UcA G*U*G*U*As*Us*Gs*Cs*C* 3′;
(SEQ ID NO: 2)
5′ U*U*C*A*C*U* UcA G*U*G*U*As*Us*Gs*Cs*C* 3′;
(SEQ ID NO: 3)
5′ A*C*C*U*C*C* AcU C*A*G*U*Gs*Us*Gs*As*U* 3′;
(SEQ ID NO: 4)
5′ U*U*U*C*C*U* CcA C*U*G*U*Us*Gs*Cs*As*A* 3′;
(SEQ ID NO: 5)
5′ U*G*U*G*U*A* UcU U*G*C*U*Gs*Us*Gs*As*G* 3′;
(SEQ ID NO: 6)
5′ G*A*G*G*U*C* CcU G*G*G*G*Gs*Cs*Gs*Cs*U* 3′;
(SEQ ID NO: 7)
5′ G*A*U*C*U*U* CcU G*A*U*G*Gs*Cs*Cs*As*C* 3′;
(SEQ ID NO: 8)
5′ A*G*C*C*A*C* AcA C*U*C*C*Gs*Us*Cs*As*G* 3′;
(SEQ ID NO: 9)
5′ G*A*U*U*U*U* CcU G*A*U*A*Gs*Cs*Us*As*C* 3′;
(SEQ ID NO: 10)
5′ G*G*C*C*A*C* AcA U*U*C*U*Gs*Us*Cs*As*G* 3′;
(SEQ ID NO: 11)
5′ G*A*U*C*U*U* CcU G*A*U*G*Gs*Cs*Cs*As*C* 3′;
(SEQ ID NO: 12)
5′ G*G*C*C*A*C* AcA C*U*C*C*Gs*Us*Cs*As*G* 3′;
(SEQ ID NO: 13)
5′ G*A*U*U*U*U* CcU G*A*U*A*Gs*Cs*As*As*C* 3′;
(SEQ ID NO: 14)
5′ G*G*C*U*A*C* GcA C*U*C*U*Gs*Us*Cs*As*A* 3′;
(SEQ ID NO: 15)
5′ A*G*G*C*C*G* CcG U*C*G*U*Gs*Gs*Cs*Gs*G* 3′;
(SEQ ID NO: 16)
5′ C*C*G*C*U*C* CcU CcU C*A*G*C*Cs*Cs*Gs*Us*
C* 3′;
(SEQ ID NO: 17)
5′ A*C*G*C*C*A* CcA G*C*U*C*Cs*As*As*Cs*U* 3′;
(SEQ ID NO: 18)
5′ G*U*C*U*C*A* CcA A*U*U*G*Cs*Us*Cs*Us*C* 3′;
(SEQ ID NO: 19)
5′ G*A*A*A*U*A* CcA U*C*A*G*As*Us*Us*Us*G* 3′;
(SEQ ID NO: 20)
5′ A*A*U*U*A*G* CcU U*C*U*G*Gs*Cs*Cs*As*U* 3′;
(SEQ ID NO: 21)
5′ G*A*U*C*A*G* CcU C*C*U*G*Gs*Cs*Cs*As*U* 3′;
(SEQ ID NO: 22)
5′ G*A*U*C*A*G* CcU U*C*U*G*Gs*Cs*Cs*As*U* 3′;
(SEQ ID NO: 23)
5′ G*A*U*C*A*G* CcU U*C*U*G*Gs*Cs*Cs*As*U* 3′;
(SEQ ID NO: 24)
5′ C*A*C*U*G*C* CcA G*G*C*A*Us*Cs*As*Gs*C* 3′;
(SEQ ID NO: 25)
5′ C*A*C*U*G*C* CcG G*G*C*A*Us*Cs*As*Gs*C* 3′;
(SEQ ID NO: 26)
5′ U*C*C*G*C*C* CcG A*U*C*C*As*Cs*Gs*As*U* 3′;
(SEQ ID NO: 27)
5′ C*C*U*U*U*C* UcG U*C*G*A*Us*Gs*Gs*Us*C* 3′;
(SEQ ID NO: 28)
5′ C*C*U*U*U*C* U*cG U*C*G*A*Us*Gs*Gs*Us*C* 3′;
(SEQ ID NO: 29)
5′ C*U*U*G*A*U* AcA U*C*C*A*Gs*Us*Us*Cs*C* 3′;
(SEQ ID NO: 30)
5′ U*U*U*C*A*G* GcA U*U*U*C*Cs*Us*Cs*Cs*G* 3′;
(SEQ ID NO: 31)
5′ C*U*U*C*A*G* GcA U*G*G*G*Gs*Cs*As*Gs*C* 3′;
(SEQ ID NO: 32)
5′ A*G*G*A*A*C* AcA A*C*C*U*Us*Us*Gs*Us*C* 3′;
(SEQ ID NO: 33)
5′ U*U*U*C*A*C* AcA U*C*C*A*Us*Cs*As*As*C* 3′;
(SEQ ID NO: 34)
5′ C*U*U*C*A*C* GcA U*C*C*A*Us*Cs*As*As*C* 3′;
(SEQ ID NO: 35)
5′ U*G*G*G*A*C* AcA A*C*C*C*Cs*Us*Gs*Cs*C* 3′;
(SEQ ID NO: 36)
5′ C*G*A*C*U*C* CcU C*U*G*G*As*Us*Gs*Us*U* 3′;
(SEQ ID NO: 37)
5′ C*G*A*C*U*C* UcU C*U*G*G*As*Us*Gs*Us*U* 3′;
or a fragment or variant of any of these nucleic acid sequences;
wherein
A is an adenosine nucleotide or a variant thereof, preferably an adenosine ribonucleotide, an adenosine deoxynucleotide, a modified adenosine ribonucleotide or a modified adenosine deoxynucleotide;
C is a cytidine nucleotide or a variant thereof, preferably a cytidine ribonucleotide, a cytidine deoxynucleotide, a modified cytidine ribonucleotide or a modified cytidine deoxynucleotide;
G is a guanosine nucleotide or a variant thereof, preferably a guanosine ribonucleotide, a guanosine deoxynucleotide, a modified guanosine ribonucleotide or a modified guanosine deoxynucleotide;
U is an uridine nucleotide or a variant thereof, preferably an uridine ribonucleotide, an uridine deoxynucleotide, a modified uridine ribonucleotide or a modified uridine deoxynucleotide;
As, Cs, Gs and Us are nucleotides or variants thereof, preferably ribonucleotides or deoxynucleotides as defined above, further comprising a phosphorothioate group;
wherein an asterisk (*) indicates a chemical modification of the preceding nucleotide at the 2′ carbon atom, preferably with 2′-hydrogen, 2′-O-methyl, 2′-O-methoxyethyl or 2′-fluoro; and
wherein a lower case letter c indicates the position corresponding to a nucleotide or a variant thereof, preferably an adenosine or cytidine, more preferably an adenosine, to be edited in the target sequence and wherein c represents a cytidine nucleotide or a variant thereof, a deoxycytidine nucleotide or a variant thereof, or an abasic site.

19. The artificial nucleic acid according to any of the preceding claims, wherein the targeting sequence comprises at the position corresponding to a nucleotide to be edited in the target sequence a cytidine nucleotide or a variant thereof, a deoxycytidine nucleotide or a variant thereof, or an abasic site, and
wherein at least one, preferably both, of the two nucleotides or variants thereof, which are positioned 5′ or 3′ of the position corresponding to a nucleotide to be edited in the target sequence, is chemically modified at the 2′ carbon atom, wherein the 2′ carbon atom is linked to a substituent selected from the group consisting of a halogen, an alkoxy group, a hydrogen, an aryloxy group, an amino group and an aminoalkoxy group, preferably selected from 2′-O-methyl, 2′-O-methoxyethyl, 2′-hydrogen and 2′-fluoro; and/or
wherein at least one, preferably both, of the two nucleotides or variants thereof, which are positioned 5′ or 3′ of the position corresponding to a nucleotide to be edited in the target sequence, comprises a modified phosphate group, preferably a phosphorothioate group.

20. The artificial nucleic acid according to any of the preceding claims, wherein the targeting sequence comprises at the position corresponding to a nucleotide to be edited in the target sequence a cytidine nucleotide or a variant thereof, a deoxycytidine nucleotide or a variant thereof, or an abasic site, and
wherein the nucleotide, which is positioned 5′ of the position corresponding to the nucleotide to be edited, is a pyrimidine nucleotide, preferably a pyrimidine ribonucleotide or a pyrimidine deoxynucleotide, and wherein said pyrimidine nucleotide comprises a nucleobase, which is chemically modified at the 2′ position, preferably by 2′-hydrogen, 2′-O-methyl, 2′-O-methoxyethyl or 2′-O-fluoro.

21. The artificial nucleic acid according to any of the preceding claims, wherein the targeting sequence comprises a nucleic acid sequence as defined in any of claims 9 to 11.

22. The artificial nucleic acid according to any of the preceding claims, wherein the recruiting moiety comprises at least one coupling agent capable of recruiting a deaminase comprising a moiety that binds to said coupling agent, wherein the coupling agent is preferably covalently linked to the 5′-terminus or to the 3′-terminus of the targeting sequence or to an internal nucleotide within the targeting sequence.

23. The artificial nucleic acid according to claim 22, wherein the coupling agent is selected from the group consisting of O6-benzylguanine, O2-benzylcytosine, chloroalkane, 1×BG, 2×BG, 4×BG, and a variant of any of these.

24. The artificial nucleic acid according to claim 22 or 23, wherein the moiety binding to said coupling agent is selected from the group consisting of a SNAP-tag, a CLIP-tag, a HaloTag, and a fragment or variant of any one of these.

25. The artificial nucleic acid according to any of the preceding claims, wherein
the recruiting moiety comprises 06-benzylguanine, 1×BG, 2×BG, 4×BG or a variant of any one of these and the deaminase, preferably an adenosine deaminase, comprises a SNAP-tag or a fragment or variant thereof;
the recruiting moiety comprises a chloroalkane and the deaminase, preferably an adenosine deaminase, comprises a HaloTag or a fragment or variant thereof; or
the recruiting moiety comprises O2-benzylcytosine or a variant thereof and the deaminase, preferably an adenosine deaminase, comprises a Clip-tag or a fragment or variant thereof.

26. The artificial nucleic acid according to any of the preceding claims, wherein
the recruiting moiety comprises a coupling agent, which is capable of recruiting more than one deaminase molecule, wherein the coupling agent is preferably selected from 2×BG, 4×BG and a variant of any one of these;
and/or
the recruiting moiety comprises at least two moieties of a coupling agent, wherein the at least two moieties represent the same coupling agent or a different coupling agent.

27. The artificial nucleic acid according to any one of claims 1 to 21, wherein the recruiting moiety comprises a nucleic acid sequence capable of specifically binding to the deaminase, preferably an adenosine or cytidine deaminase.

28. The artificial nucleic acid according to claim 27, wherein the recruiting moiety comprises a nucleic acid sequence capable of specifically binding to the dsRNA binding domain of the deaminase, preferably an adenosine or cytidine deaminase.

29. The artificial nucleic acid according to any one of claims 1 to 21, 27 and 28, wherein the recruiting moiety comprises a nucleic acid sequence that is capable of intramolecular base pairing, preferably capable of forming a stem-loop structure.

30. The artificial nucleic acid according to claim 29, wherein the stem-loop structure comprises a double-helical stem comprising at least two mismatches.

31. The artificial nucleic acid according to claim 29 or 30, wherein the stem loop structure comprises a loop consisting of from 3 to 8, preferably from 4 to 6, more preferably 5, nucleotides, wherein the loop preferably comprises the nucleic acid sequence GCUAA or GCUCA.

32. The artificial nucleic acid according to any of claims 1 to 21 and 27 to 31, wherein the recruiting moiety comprises a nucleic acid sequence comprising at least one nucleotide,
wherein the nucleobase is chemically modified, and/or
wherein the nucleic acid sequence comprises at least one backbone modification.

33. The artificial nucleic acid according to claim 32, wherein the recruiting moiety comprises a nucleic acid sequence comprising at least one chemically modified nucleotide, which is chemically modified at the 2′ position.

34. The artificial nucleic acid according to claim 33, wherein
the chemically modified nucleotide comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro; and/or
wherein the chemically modified nucleotide is a locked nucleic acid (LNA) nucleotide, an ethylene bridged nucleic acid (ENA) nucleotide or an (S)-constrained ethyl cEt nucleotide.

35. The artificial nucleic acid according to any of claims 32 to 34, wherein the recruiting moiety comprises a nucleic acid sequence comprising at least one backbone modification and wherein the phosphate group linking the sugars of two neighbouring nucleotides is a modified phosphate group, preferably selected from the group consisting of a phosphorothioate, a phosphoroselenate, a borano phosphate, a borano phosphate ester, a hydrogen phosphonate, a phosphoroamidate, an alkyl phosphonate, an aryl phosphonate and a phosphotriester.

36. The artificial nucleic acid according to any of claims 32 to 35, wherein the recruiting moiety comprises a nucleic acid sequence, wherein at least 40% of the nucleotides are chemically modified at the 2′ position.

37. The artificial nucleic acid according to any of claims 32 to 36, wherein the recruiting moiety comprises a nucleic acid sequence, wherein at least of two of the five nucleotides at the 5′ terminus of the nucleic acid sequence comprise a phosphorothioate group.

38. The artificial nucleic acid according to any of claims 32 to 37, wherein the recruiting moiety comprises a nucleic acid sequence, wherein at least of two of the five nucleotides at the 5′ terminus of the nucleic acid sequence are LNA nucleotides, ENA nucleotides or (S)-constrained ethyl cEt nucleotides.

39. The artificial nucleic acid according to any of claims 32 to 38, wherein the recruiting moiety comprises a nucleic acid sequence comprising at least one nucleotide comprising a modified phosphate group, preferably a phosphorothioate group;
at least one LNA nucleotide, ENA nucleotide or (S)-constrained ethyl cEt nucleotide; and
at least one nucleotide comprising a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro.

40. The artificial nucleic acid according to any of claims 1 to 21 and 27 to 39, wherein the recruiting moiety comprises a nucleic acid sequence selected from the group consisting of
(a)
(SEQ ID NO: 38)
5′ GGUGUCGAG-Na-AGA-N_{c}-GAGAACAAUAU-G
CU A/C A-AUGUUGUUCUC-N_{d}-UCU-N_{b}-CUCGA
CACC 3′;
(b)
(SEQ ID NO: 39)
5′ GsGsUGUCGAG-Nₐ-AGA-N_{c}-GAGAACAAUAU-
GCU A/C A-AUGUUGUUCUC-N_{d}-UCU-N_{b}-CUCGA
CACC 3′;
and
(c)
(SEQ ID NO: 40)
5′ GslGslUGUCGAG-Nₐ-AGA-N_{c}-GAGAACAAU
AU-GCU A/C A-AUGUUGUUCUC-N_{d}-UCU-N_{b}-CU
CGACACC 3′;
or a fragment or variant of any of these nucleic acid sequences;
wherein
Nₐ and N_{b} form a mismatch, preferably wherein Nₐ is adenosine and N_{b} is cytidine;
N_{c} and N_{d} form a mismatch, preferably wherein N_{c} and N_{d} are guanosine;
Gs is a guanosine comprising a phosphorothioate group; and
GsI is an LNA guanosine comprising a phosphorothioate group.

41. The artificial nucleic acid according to any of claims 1 to 21 and 27 to 39, wherein the recruiting moiety comprises a nucleic acid sequence derived from VA RNA I, or a fragment or variant thereof.

42. The artificial nucleic acid according to any of claims 1 to 21, 27 to 39 and 41, wherein the recruiting moiety comprises the nucleic acid sequence
(SEQ ID NO: 41)
GCACACCTGGGTTCGACACGCGGGCGGTAACCGCATG
GATCACGGCGGACGGCCGGATTCGGGGTTCGAACCCC
GGTCGTCCGCCATGATACCCTTGC,
or a fragment or variant thereof.

43. The artificial nucleic acid according to any of claims 40 to 42, wherein the recruiting moiety comprises a nucleic acid sequence as defined in said claims, wherein at least one nucleotide, preferably at least 40%,50%, 60%, 70%, 80%, 90%, 95%, 99% or 100% of the nucleotides, comprises a substituent at the 2′ carbon atom, wherein the substituent is selected from the group consisting of a halogen, an alkoxy group, a hydrogen, an aryloxy group, an amino group and an aminoalkoxy group, preferably from 2′-hydrogen, 2′-O-methyl, 2′-O-methoxyethyl and 2′-fluoro.

44. The artificial nucleic acid according to any of the preceding claims, wherein the recruiting moiety comprises a nucleic acid sequence selected from the group consisting of
(a)
(SEQ ID NO: 42)
5′ G*G*U*GU*C*GAG-Nₐ-AGA-N_{c}-GAGAAC*AA
U*AU*-GC*U* A/C A- AU*GU*U*GU*U*C*U*
C*-N_{d}-U*C*U*-N_{b}*-C*U*C*GAC*AC*C* 3′;
(b)
(SEQ ID NO: 43)
5′ Gs*Gs*U*GU*C*GAG-Nₐ-AGA-N_{c}-GAGAAC*
AAU*AU*-GC*U* A/C A-AU*GU*U*GU*U*C*U*
C*-N_{d}-U*C*U*-N_{b}*-C*U*C*GAC*AC*C* 3′;
and
(c)
(SEQ ID NO: 44)
5′ Gsl*Gsl*U*GU*C*GAG-Nₐ-AGA-N_{c}-GAGA
AC*AAU*AU*-GC*U* A/C A-AU*GU*U*GU*U*C
*U*C*-N_{d}-U*CU*-N_{b}*-C*U*C*GAC*AC*C* 3′;
or a fragment or variant of any of these sequences;
wherein
Nₐ and N_{b} form a mismatch, preferably wherein Nₐ is adenosine and N_{b} is cytidine;
N_{c} and N_{d} form a mismatch, preferably wherein N_{c} and N_{d} are guanosine;
Gs is a guanosine comprising a phosphorothioate group;
GsI is an LNA guanosine comprising a phosphorothioate group; and
wherein an asterisk (*) indicates a modification of the nucleotide at the 2′ carbon atom, preferably with 2′-hydrogen, 2′-O-methyl, 2′-O-methoxyethyl or 2′-fluoro.

45. Artificial nucleic acid for site-directed editing of a target RNA, the artificial nucleic acid comprising
a) a targeting sequence, which comprises or consists of a nucleic acid sequence complementary or partially complementary to a target sequence in the target RNA, and
b) a recruiting moiety for recruiting a deaminase, wherein the recruiting moiety comprises a nucleic acid sequence capable of specifically binding to the deaminase, preferably an adenosine or cytidine deaminase,
wherein the recruiting moiety is characterized by any one of the features defined in claims 27 to 44.

46. The artificial nucleic acid according to any of the preceding claims, which further comprises a moiety, which enhances cellular uptake of the artificial nucleic acid.

47. The artificial nucleic acid according to claim 46, wherein the moiety enhancing cellular uptake is a triantennary N-acetyl galactosamine (GalNAc3), which is preferably conjugated with the 3′ terminus or with the 5′ terminus of the artificial nucleic acid.

48. The artificial nucleic acid according to any of the preceding claims, comprising in 5′ to 3′ direction the recruiting moiety and the targeting sequence defined in the preceding claims.

49. The artificial nucleic acid according to any of the preceding claims, which is an RNA.

50. The artificial nucleic acid according to any of the preceding claims, wherein the deaminase is
an adenosine deaminase or a fragment or variant thereof, preferably selected from the group consisting of ADAR1, ADAR2 and a fragment or variant thereof, more preferably a peptide or protein comprising an adenosine deaminase domain; or
a cytidine deaminase or a fragment or variant thereof, preferably Apobec1 or a fragment or variant thereof, more preferably a peptide or protein comprising a cytidine deaminase domain.

51. The artificial nucleic acid according to any of the preceding claims, wherein the deaminase is an adenosine deaminase, preferably a eukaryotic adenosine deaminase, more preferably a vertebrate adenosine deaminase, even more preferably a mammalian adenosine deaminase, most preferably a human adenosine deaminase, or
a cytidine deaminase, preferably a eukaryotic cytidine deaminase, more preferably a vertebrate cytidine deaminase, even more preferably a mammalian cytidine deaminase, even more preferably a murine or a human cytidine deaminase, most preferably mApobec1.

52. The artificial nucleic acid according to any of the preceding claims, wherein the site-directed editing comprises the deamination of adenosine or cytidine in the target sequence.

53. Vector encoding the artificial nucleic acid according to any of the preceding claims.

54. Cell comprising the artificial nucleic acid according to any of claims 1 to 52 or the vector according to claim 53.

55. Composition comprising the artificial nucleic acid according to any one of claims 1 to 52, the vector according to claim 53 or the cell according to claim 54, and an additional excipient, preferably a pharmaceutically acceptable excipient.

56. The composition according to claim 55 comprising the artificial nucleic acid or the vector in the form of a nanoparticle, preferably a lipid nanoparticle or a liposome.

57. The composition according to claim 55 or 56, wherein the artificial nucleic acid or the vector is complexed by a cationic compound.

58. The composition according to claim 57, wherein the cationic compound is a cationic lipid.

59. Kit comprising the artificial nucleic acid according to any one of claims 1 to 52, the vector according to claim 53, the cell according to claim 54, or the composition according to any of claims 55 to 58.

60. Use of the artificial nucleic acid according to any of claims 1 to 52, the vector according to claim 53, the cell according to claim 54, the composition according to any of claims 55 to 58 or the kit according to claim 59 for site-directed editing of a target RNA.

61. Use of the artificial nucleic acid according to any of claims 1 to 52, the vector according to claim 53, the cell according to claim 54, the composition according to any of claims 55 to 58 or the kit according to claim 59 for in vitro diagnosis of a disease or disorder.

62. Method for site-directed editing of a target RNA, which comprises contacting a target RNA with the artificial nucleic acid according to any of claims 1 to 52.

63. The artificial nucleic acid according to any of claims 1 to 52, the vector according to claim 53, the cell according to claim 54, the composition according to any of claims 55 to 58 or the kit according to claim 59 for use as a medicament.

64. The artificial nucleic acid according to any of claims 1 to 52, the vector according to claim 53, the cell according to claim 54, the composition according to any of claims 55 to 58 or the kit according to claim 59 for use in the treatment or prophylaxis of a disease or disorder selected from the group consisting of infectious diseases, tumour diseases, cardiovascular diseases, autoimmune diseases, allergies and neurological diseases or disorders.

65. The artificial nucleic acid according to any of claims 1 to 52, the vector according to claim 53, the cell according to claim 54, the composition according to any of claims 55 to 58 or the kit according to claim 59 for use in the treatment or prophylaxis of a disease or disorder, wherein the treatment or prophylaxis comprises a step of site-directed editing of a target RNA.

66. The artificial nucleic acid according to any of claims 1 to 52, the vector according to claim 53, the cell according to claim 54, the composition according to any of claims 55 to 58 or the kit according to claim 59 for use in the diagnosis of a disease or disorder, which is preferably selected from the group consisting of infectious diseases, tumour diseases, cardiovascular diseases, autoimmune diseases, allergies and neurological diseases or disorders.

67. Method for treating a subject with a disease or a disorder, the method comprising administering an effective amount of the artificial nucleic acid according to any of claims 1 to 52, the vector according to claim 53, the cell according to claim 54, or the composition according to any of claims 55 to 58 to the subject.

68. The method according to claim 67, wherein the disease or the disorder is selected from the group consisting of infectious diseases, tumour diseases, cardiovascular diseases, autoimmune diseases, allergies and neurological diseases or disorders.


SEQUENCE LISTING (128 sequences)

SEQ ID NO: 1 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uucacuunaguguaugcc

SEQ ID NO: 2 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uucacuunaguguaugcc

SEQ ID NO: 3 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
accuccanucagugugau

SEQ ID NO: 4 - DNA, 18 bp, Artificial Sequence
  target sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uuuccucnacuguugcaa

SEQ ID NO: 5 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uguguaunuugcugugag

SEQ ID NO: 6 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gagguccnugggggcgcu

SEQ ID NO: 7 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaucuucnugauggccac

SEQ ID NO: 8 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
agccacanacuccgucag

SEQ ID NO: 9 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gauuuucnugauagcuac

SEQ ID NO: 10 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ggccacanauucugucag

SEQ ID NO: 11 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaucuucnugauggccac

SEQ ID NO: 12 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ggccacanacuccgucag

SEQ ID NO: 13 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gauuuucnugauagcaac

SEQ ID NO: 14 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ggcuacgnacucugucaa

SEQ ID NO: 15 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
aggccgcngucguggcgg

SEQ ID NO: 16 - DNA, 21 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (11)..(11): n is a cytidine nucleotide or a variant thereof, a deoxycytidine nucleotide or a variant thereo f, or an abasic site
  misc_feature (13)..(21): chemical modification at the 2′ carbon atom, preferably with 2′-hydrogen (2′-deoxy), 2′-O-methyl, 2′-O-methoxyethyl or 2′-fluoro
  misc_feature (17)..(20): phosphorothioate modification
ccgcuccnucnucagcccguc

SEQ ID NO: 17 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
acgccacnagcuccaacu

SEQ ID NO: 18 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gucucacnaauugcucuc

SEQ ID NO: 19 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaaauacnaucagauuug

SEQ ID NO: 20 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
aauuagcnuucuggccau

SEQ ID NO: 21 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaucagcnuccuggccau

SEQ ID NO: 22 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaucagcnuucuggccau

SEQ ID NO: 23 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
gaucagcnuucuggccau

SEQ ID NO: 24 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cacugccnaggcaucagc

SEQ ID NO: 25 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cacugccngggcaucagc

SEQ ID NO: 26 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uccgcccngauccacgau

SEQ ID NO: 27 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ccuuucungucgaugguc

SEQ ID NO: 28 - DNA, 18 bp, Artificial Sequence
  targeting sequence
  misc_feature (1)..(7): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ccuuucungucgaugguc

SEQ ID NO: 29 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cuugauanauccaguucc

SEQ ID NO: 30 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uuucaggnauuuccuccg

SEQ ID NO: 31 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cuucaggnauggggcagc

SEQ ID NO: 32 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
aggaacanaaccuuuguc

SEQ ID NO: 33 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
uuucacanauccaucaac

SEQ ID NO: 34 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cuucacgnauccaucaac

SEQ ID NO: 35 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
ugggacanaaccccugcc

SEQ ID NO: 36 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cgacuccnucuggauguu

SEQ ID NO: 37 - DNA, 18 bp, Artificial Sequence
  targeting sequence
  misc_feature (1)..(6): chemical modification at the 2′ carbon atom, preferably with 2′-hydrogen (2′-deoxy), 2′-O-methyl, 2′-O-methoxyethyl or 2′-fluoro
  misc_feature (8)..(8): n is a cytidine nucleotide or a variant thereof, a deoxycytidine nucleotide or a variant thereo f, or an abasic site
  misc_feature (10)..(18): chemical modification at the 2′ carbon atom, preferably with 2′-hydrogen (2′-deoxy), 2′-O-methyl, 2′-O-methoxyethyl or 2′-fluoro
  misc_feature (14)..(17): phosphorothioate modification
cgacucunucuggauguu

SEQ ID NO: 38 - DNA, 55 bp, Artificial Sequence
  recruiting moiety
  misc_feature (10)..(10): n is a, c, g, t or u
  misc_feature (14)..(14): n is a, c, g, t or u
  misc_feature (42)..(42): n is a, c, g, t or u
  misc_feature (46)..(46): n is a, c, g, t or u
ggugucgagnagangagaacaauaugcuhaauguuguucucnucuncucgacacc

SEQ ID NO: 39 - DNA, 55 bp, Artificial Sequence; identical to SEQ ID NO: 38
  recruiting moiety
  misc_feature (1)..(2): phosphorothioate modification
  misc_feature (10)..(10): n is a, c, g, t or u
  misc_feature (14)..(14): n is a, c, g, t or u
  misc_feature (42)..(42): n is a, c, g, t or u
  misc_feature (46)..(46): n is a, c, g, t or u

SEQ ID NO: 40 - DNA, 55 bp, Artificial Sequence; identical to SEQ ID NO: 38
  recruiting moiety
  misc_feature (1)..(2): LNA guanosine comprising a phosphorothioate group
  misc_feature (10)..(10): n is a, c, g, t or u
  misc_feature (14)..(14): n is a, c, g, t or u
  misc_feature (42)..(42): n is a, c, g, t or u
  misc_feature (46)..(46): n is a, c, g, t or u

SEQ ID NO: 41 - DNA, 98 bp, Artificial Sequence
  recruiting moiety
gcacacctgggttcgacacgcgggcggtaaccgcatggatcacggcggacggccggattcggggttcgaaccccggtcgtccgccatgatacccttgc

SEQ ID NO: 42 - DNA, 55 bp, Artificial Sequence; identical to SEQ ID NO: 38
  recruiting moiety
  misc_feature (1)..(3): chemical modification at the 2′ carbon atom, preferably with 2′-hydrogen (2′-deoxy), 2′-O-methyl, 2′-O-methoxyethyl or 2′-fluoro
  misc_feature (5)..(6): chemical modification at the 2′ carbon atom, preferably with 2′-hydrogen (2′-deoxy), 2′-O-methyl, 2′-O-methoxyethyl or 2′-fluoro
  misc_feature (10)..(10): n is a, c, g, t or u
  misc_feature (14)..(14): n is a, c, g, t or u
  misc_feature (20)..(20): chemical modification at the 2′ carbon atom, preferably with 2′-hydrogen (2′-deoxy), 2′-O-methyl, 2′-O-methoxyethyl or 2′-fluoro
  misc_feature (23)..(23): chemical modification at the 2′ carbon atom, preferably with 2′-hydrogen (2′-deoxy), 2′-O-methyl, 2′-O-methoxyethyl or 2′-fluoro
  misc_feature (25)..(25): chemical modification at the 2′ carbon atom, preferably with 2′-hydrogen (2′-deoxy), 2′-O-methyl, 2′-O-methoxyethyl or 2′-fluoro
  misc_feature (27)..(28): chemical modification at the 2′ carbon atom, preferably with 2′-hydrogen (2′-deoxy), 2′-O-methyl, 2′-O-methoxyethyl or 2′-fluoro
  misc_feature (32)..(32): chemical modification at the 2′ carbon atom, preferably with 2′-hydrogen (2′-deoxy), 2′-O-methyl, 2′-O-methoxyethyl or 2′-fluoro
  misc_feature (34)..(35): chemical modification at the 2′ carbon atom, preferably with 2′-hydrogen (2′-deoxy), 2′-O-methyl, 2′-O-methoxyethyl or 2′-fluoro
  misc_feature (37)..(41): chemical modification at the 2′ carbon atom, preferably with 2′-hydrogen (2′-deoxy), 2′-O-methyl, 2′-O-methoxyethyl or 2′-fluoro
  misc_feature (42)..(42): n is a, c, g, t or u
  misc_feature (43)..(49): chemical modification at the 2′ carbon atom, preferably with 2′-hydrogen (2′-deoxy), 2′-O-methyl, 2′-O-methoxyethyl or 2′-fluoro
  misc_feature (52)..(52): chemical modification at the 2′ carbon atom, preferably with 2′-hydrogen (2′-deoxy), 2′-O-methyl, 2′-O-methoxyethyl or 2′-fluoro
  misc_feature (54)..(55): chemical modification at the 2′ carbon atom, preferably with 2′-hydrogen (2′-deoxy), 2′-O-methyl, 2′-O-methoxyethyl or 2′-fluoro

SEQ ID NO: 43 - DNA, 55 bp, Artificial Sequence; identical to SEQ ID NO: 38
  recruiting moiety
  misc_feature (1)..(2): phosphorothioate modification
  misc_feature (1)..(3): chemical modification at the 2′ carbon atom, preferably with 2′-hydrogen (2′-deoxy), 2′-O-methyl, 2′-O-methoxyethyl or 2′-fluoro
  misc_feature (5)..(6): chemical modification at the 2′ carbon atom, preferably with 2′-hydrogen (2′-deoxy), 2′-O-methyl, 2′-O-methoxyethyl or 2′-fluoro
  misc_feature (10)..(10): n is a, c, g, t or u
  misc_feature (14)..(14): n is a, c, g, t or u
  misc_feature (20)..(20): chemical modification at the 2′ carbon atom, preferably with 2′-hydrogen (2′-deoxy), 2′-O-methyl, 2′-O-methoxyethyl or 2′-fluoro
  misc_feature (23)..(23): chemical modification at the 2′ carbon atom, preferably with 2′-hydrogen (2′-deoxy), 2′-O-methyl, 2′-O-methoxyethyl or 2′-fluoro
  misc_feature (25)..(25): chemical modification at the 2′ carbon atom, preferably with 2′-hydrogen (2′-deoxy), 2′-O-methyl, 2′-O-methoxyethyl or 2′-fluoro
  misc_feature (27)..(28): chemical modification at the 2′ carbon atom, preferably with 2′-hydrogen (2′-deoxy), 2′-O-methyl, 2′-O-methoxyethyl or 2′-fluoro
  misc_feature (32)..(32): chemical modification at the 2′ carbon atom, preferably with 2′-hydrogen (2′-deoxy), 2′-O-methyl, 2′-O-methoxyethyl or 2′-fluoro
  misc_feature (34)..(35): chemical modification at the 2′ carbon atom, preferably with 2′-hydrogen (2′-deoxy), 2′-O-methyl, 2′-O-methoxyethyl or 2′-fluoro
  misc_feature (37)..(41): chemical modification at the 2′ carbon atom, preferably with 2′-hydrogen (2′-deoxy), 2′-O-methyl, 2′-O-methoxyethyl or 2′-fluoro
  misc_feature (42)..(42): n is a, c, g, t or u
  misc_feature (43)..(49): chemical modification at the 2′ carbon atom, preferably with 2′-hydrogen (2′-deoxy), 2′-O-methyl, 2′-O-methoxyethyl or 2′-fluoro
  misc_feature (52)..(52): chemical modification at the 2′ carbon atom, preferably with 2′-hydrogen (2′-deoxy), 2′-O-methyl, 2′-O-methoxyethyl or 2′-fluoro
  misc_feature (54)..(55): chemical modification at the 2′ carbon atom, preferably with 2′-hydrogen (2′-deoxy), 2′-O-methyl, 2′-O-methoxyethyl or 2′-fluoro

SEQ ID NO: 44 - DNA, 55 bp, Artificial Sequence; identical to SEQ ID NO: 38
  recruiting moiety
  misc_feature (1)..(1): LNA guanosine comprising a phosphorothioate group
  misc_feature (1)..(3): chemical modification at the 2′ carbon atom, preferably with 2′-hydrogen (2′-deoxy), 2′-O-methyl, 2′-O-methoxyethyl or 2′-fluoro
  misc_feature (5)..(6): chemical modification at the 2′ carbon atom, preferably with 2′-hydrogen (2′-deoxy), 2′-O-methyl, 2′-O-methoxyethyl or 2′-fluoro
  misc_feature (10)..(10): n is a, c, g, t or u
  misc_feature (14)..(14): n is a, c, g, t or u
  misc_feature (20)..(20): chemical modification at the 2′ carbon atom, preferably with 2′-hydrogen (2′-deoxy), 2′-O-methyl, 2′-O-methoxyethyl or 2′-fluoro
  misc_feature (23)..(23): chemical modification at the 2′ carbon atom, preferably with 2′-hydrogen (2′-deoxy), 2′-O-methyl, 2′-O-methoxyethyl or 2′-fluoro
  misc_feature (25)..(25): chemical modification at the 2′ carbon atom, preferably with 2′-hydrogen (2′-deoxy), 2′-O-methyl, 2′-O-methoxyethyl or 2′-fluoro
  misc_feature (27)..(28): chemical modification at the 2′ carbon atom, preferably with 2′-hydrogen (2′-deoxy), 2′-O-methyl, 2′-O-methoxyethyl or 2′-fluoro
  misc_feature (32)..(32): chemical modification at the 2′ carbon atom, preferably with 2′-hydrogen (2′-deoxy), 2′-O-methyl, 2′-O-methoxyethyl or 2′-fluoro
  misc_feature (34)..(35): chemical modification at the 2′ carbon atom, preferably with 2′-hydrogen (2′-deoxy), 2′-O-methyl, 2′-O-methoxyethyl or 2′-fluoro
  misc_feature (37)..(41): chemical modification at the 2′ carbon atom, preferably with 2′-hydrogen (2′-deoxy), 2′-O-methyl, 2′-O-methoxyethyl or 2′-fluoro
  misc_feature (42)..(42): n is a, c, g, t or u
  misc_feature (43)..(49): chemical modification at the 2′ carbon atom, preferably with 2′-hydrogen (2′-deoxy), 2′-O-methyl, 2′-O-methoxyethyl or 2′-fluoro
  misc_feature (52)..(52): chemical modification at the 2′ carbon atom, preferably with 2′-hydrogen (2′-deoxy), 2′-O-methyl, 2′-O-methoxyethyl or 2′-fluoro
  misc_feature (54)..(55): chemical modification at the 2′ carbon atom, preferably with 2′-hydrogen (2′-deoxy), 2′-O-methyl, 2′-O-methoxyethyl or 2′-fluoro

SEQ ID NO: 45 - DNA, 2796 bp, Homo sapiens
atggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatag

SEQ ID NO: 46 - protein, 931 aa, Homo sapiens
MAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 47 - DNA, 2796 bp, Homo sapiens; identical to SEQ ID NO: 45

SEQ ID NO: 48 - protein, 931 aa, Homo sapiens; identical to SEQ ID NO: 46

SEQ ID NO: 49 - DNA, 2100 bp, Homo sapiens
atggatatagaagatgaagaaaacatgagttccagcagcactgatgtgaaggaaaaccgcaatctggacaacgtgtcccccaaggatggcagcacacctgggcctggcgagggctctcagctctccaatgggggtggtggtggccccggcagaaagcggcccctggaggagggcagcaatggccactccaagtaccgcctgaagaaaaggaggaaaacaccagggcccgtcctccccaagaacgccctgatgcagctgaatgagatcaagcctggtttgcagtacacactcctgtcccagactgggcccgtgcacgcgcctttgtttgtcatgtctgtggaggtgaatggccaggtttttgagggctctggtcccacaaagaaaaaggcaaaactccatgctgctgagaaggccttgaggtctttcgttcagtttcctaatgcctctgaggcccacctggccatggggaggaccctgtctgtcaacacggacttcacatctgaccaggccgacttccctgacacgctcttcaatggttttgaaactcctgacaaggcggagcctcccttttacgtgggctccaatggggatgactccttcagttccagcggggacctcagcttgtctgcttccccggtgcctgccagcctagcccagcctcctctccctgccttaccaccattcccacccccgagtgggaagaatcccgtgatgatcttgaacgaactgcgcccaggactcaagtatgacttcctctccgagagcggggagagccatgccaagagcttcgtcatgtctgtggtcgtggatggtcagttctttgaaggctcggggagaaacaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggttccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaaacccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacg

SEQ ID NO: 50 - protein, 700 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPALPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 51 - DNA, 1872 bp, Artificial Sequence
  SNAPf-ADAR1
atggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggtcctgcaggcggaggcgcgccagggtctggcggcggcagtaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 52 - protein, 624 aa, Artificial Sequence
  SNAPf-ADAR1
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 53 - DNA, 1848 bp, Artificial Sequence
  SNAPf-ADAR2
atggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggtcctgcaggcggaggcgcgccagggtctggcggcggcagtaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccc

SEQ ID NO: 54 - protein, 616 aa, Artificial Sequence
  SNAPf-ADAR2
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGPAGGGAPGSGGGSKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 55 - DNA, 1266 bp, Mus musculus
atgagttccgagacaggccctgtagctgttgatcccactctgaggagaagaattgagccccacgagtttgaagtcttctttgacccccgggagcttcggaaagagacctgtctgctgtatgagatcaactggggtggaaggcacagtgtctggcgacacacgagccaaaacaccagcaaccacgttgaagtcaacttcttagaaaaatttactacagaaagatactttcgtccgaacaccagatgctccattacctggttcctgtcctggagtccctgcggggagtgctccagggccattacagagtttctgagccgacacccctatgtaactctgtttatttacatagcacggctttatcaccacacggatcagcgaaaccgccaaggactcagggaccttattagcagcggtgtgactatccagatcatgacagagcaagagtattgttactgctggaggaatttcgtcaactaccccccttcaaacgaagcatattggccaaggtacccccatctgtgggtgaaactgtatgtactggagctctactgcatcattttaggacttccaccctgtttaaaaattttaagaagaaagcaacctcaactcacgtttttcacaattactcttcaaacctgccattaccaaaggataccaccccatctcctttgggctacagggttgaaaggagcggccgcgactggcgcgccagggggatccatggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggt

SEQ ID NO: 56 - protein, 422 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLKGAAATGAPGGSMDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 57 - DNA, 2217 bp, Artificial Sequence
  Halo-ADAR1Q
atggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagaagccaacccctgcaggcggaggcgcgccagggtctggcggcggcagtaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggacaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 58 - protein, 739 aa, Artificial Sequence
  Halo-ADAR1Q
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEKPTPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 59 - DNA, 1872 bp, Artificial Sequence
  Clipf-ADAR1Q
atggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatccaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagcgagagccacctggccgccctggtgggcaatcccgccgccaccgccgccgtgaacaccgccctggacggaaatcccgtgcccattctgatcccctgccaccgggtggtgcagggcgacagcgacgtggggccctacctgggcgggctcgccgtgaaagagtggctgctggcccacgagggccacagactgggcaagcctgggctgggtcctgcaggcggaggcgcgccagggtctggcggcggcagtaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggacaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 60 - protein, 624 aa, Artificial Sequence
  Clipf-ADAR1Q
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLGPAGGGAPGSGGGSKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGQGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 61 - DNA, 85 bp, Artificial Sequence
  Luciferase R/G-v1
guggaauaguauaacaauaugcuaaauguuguuauaguaucccacgugcagccagccguccucuagagggcccugaagagggccc

SEQ ID NO: 62 - DNA, 85 bp, Artificial Sequence
  Luciferase R/G-v4
guggaagaggagaacaauaugcuaaauguuguucucgucucccacgugcagccagccguccucuagagggcccugaagagggccc

SEQ ID NO: 63 - DNA, 95 bp, Artificial Sequence
  Luciferase R/G-v9.4
guggucgagaagaggagaacaauaugcuaaauguuguucucgucuccucgaccacgugcagccagccguccucuagagggcccugaagagggccc

SEQ ID NO: 64 - DNA, 18 bp, Artificial Sequence
  ACTB 3′UTR 18nt
  misc_feature (1)..(6): chemical modification at the 2′ carbon atom with 2′-O-methyl
  misc_feature (10)..(18): chemical modification at the 2′ carbon atom with 2′-O-methyl
  misc_feature (14)..(17): phosphorothioate modification
gcaaugccaucaccuccc

SEQ ID NO: 65 - DNA, 63 bp, Artificial Sequence
  ACTB 3′UTR ASO v1
  misc_feature (46)..(51): chemical modification at the 2′ carbon atom with 2′-O-methyl
  misc_feature (55)..(63): chemical modification at the 2′ carbon atom with 2′-O-methyl
  misc_feature (59)..(62): phosphorothioate modification
ggugaauaguauaacaauaugcuaaauguuguuauaguauccaccgcaaugccaucaccuccc

SEQ ID NO: 66 - DNA, 63 bp, Artificial Sequence
  ACTB 3′UTR ASO v4
  misc_feature (46)..(51): chemical modification at the 2′ carbon atom with 2′-O-methyl
  misc_feature (55)..(63): chemical modification at the 2′ carbon atom with 2′-O-methyl
  misc_feature (59)..(62): phosphorothioate modification
ggugaagaggagaacaauaugcuaaauguuguucucgucuccaccgcaaugccaucaccuccc

SEQ ID NO: 67 - DNA, 73 bp, Artificial Sequence
  ACTB 3′UTR ASO v9.4
  misc_feature (56)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification
ggugucgagaagaggagaacaauaugcuaaauguuguucucgucuccucgacaccgcaaugccaucaccuccc

SEQ ID NO: 68 - DNA, 18 bp, Artificial Sequence
  GAPDH 3′UTR 18nt
  misc_feature (1)..(6): chemical modification at the 2′ carbon atom with 2′-O-methyl
  misc_feature (10)..(18): chemical modification at the 2′ carbon atom with 2′-O-methyl
  misc_feature (14)..(17): phosphorothioate modification
agggguccacauggcaac

SEQ ID NO: 69 - DNA, 63 bp, Artificial Sequence
  GAPDH 3′UTR ASO v1
  misc_feature (46)..(51): chemical modification at the 2′ carbon atom with 2′-O-methyl
  misc_feature (55)..(63): chemical modification at the 2′ carbon atom with 2′-O-methyl
  misc_feature (59)..(62): phosphorothioate modification
ggugaauaguauaacaauaugcuaaauguuguuauaguauccaccagggguccacauggcaac

SEQ ID NO: 70 - DNA, 63 bp, Artificial Sequence
  GAPDH 3′UTR ASO v4
  misc_feature (46)..(51): chemical modification at the 2′ carbon atom with 2′-O-methyl
  misc_feature (55)..(63): chemical modification at the 2′ carbon atom with 2′-O-methyl
  misc_feature (59)..(62): phosphorothioate modification
ggugaagaggagaacaauaugcuaaauguuguucucgucuccaccagggguccacauggcaac

SEQ ID NO: 71 - DNA, 73 bp, Artificial Sequence
  GAPDH 3′UTR ASO v9.4
  misc_feature (56)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification
ggugucgagaagaggagaacaauaugcuaaauguuguucucgucuccucgacaccagggguccacauggcaac

SEQ ID NO: 72 - DNA, 73 bp, Artificial Sequence; identical to SEQ ID NO: 71
  GAPDH 3′UTR ASO v9.5
  misc_feature (1)..(3): chemical modification at the 2′ carbon atom with 2′-O-methyl
  misc_feature (1)..(2): phosphorothioate modification
  misc_feature (5)..(6): chemical modification at the 2′ carbon atom with 2′-O-methyl
  misc_feature (20)..(20): chemical modification at the 2′ carbon atom with 2′-O-methyl
  misc_feature (23)..(23): chemical modification at the 2′ carbon atom with 2′-O-methyl
  misc_feature (25)..(25): chemical modification at the 2′ carbon atom with 2′-O-methyl
  misc_feature (27)..(28): chemical modification at the 2′ carbon atom with 2′-O-methyl
  misc_feature (32)..(32): chemical modification at the 2′ carbon atom with 2′-O-methyl
  misc_feature (34)..(35): chemical modification at the 2′ carbon atom with 2′-O-methyl
  misc_feature (37)..(41): chemical modification at the 2′ carbon atom with 2′-O-methyl
  misc_feature (43)..(49): chemical modification at the 2′ carbon atom with 2′-O-methyl
  misc_feature (52)..(52): chemical modification at the 2′ carbon atom with 2′-O-methyl
  misc_feature (54)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification

SEQ ID NO: 73 - DNA, 18 bp, Artificial Sequence
  GAPDH ORF1 ASO 18nt
  misc_feature (1)..(6): chemical modification at the 2′ carbon atom with 2′-O-methyl
  misc_feature (10)..(18): chemical modification at the 2′ carbon atom with 2′-O-methyl
  misc_feature (14)..(17): phosphorothioate modification
ggggugccaagcaguugg

SEQ ID NO: 74 - DNA, 73 bp, Artificial Sequence
  GAPDH ORF1 ASO v9.4
  misc_feature (56)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification
ggugucgagaagaggagaacaauaugcuaaauguuguucucgucuccucgacaccggggugccaagcaguugg

SEQ ID NO: 75 - DNA, 18 bp, Artificial Sequence
  GAPDH ORF2 ASO 18nt
  misc_feature (1)..(6): chemical modification at the 2′ carbon atom with 2′-O-methyl
  misc_feature (10)..(18): chemical modification at the 2′ carbon atom with 2′-O-methyl
  misc_feature (14)..(17): phosphorothioate modification
ggggugccaagcaguugg

SEQ ID NO: 76 - DNA, 73 bp, Artificial Sequence
  GAPDH ORF2 ASO v9.4
  misc_feature (56)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification
ggugucgagaagaggagaacaauaugcuaaauguuguucucgucuccucgacaccguuuuuccagacggcagg

SEQ ID NO: 77 - DNA, 95 bp, Artificial Sequence
  GAPDH ORF1 ASO v25
  misc_feature (1)..(3): chemical modification at the 2′ carbon atom with 2′-O-methyl
  misc_feature (1)..(2): phosphorothioate modification
  misc_feature (5)..(6): chemical modification at the 2′ carbon atom with 2′-O-methyl
  misc_feature (20)..(20): chemical modification at the 2′ carbon atom with 2′-O-methyl
  misc_feature (23)..(23): chemical modification at the 2′ carbon atom with 2′-O-methyl
  misc_feature (25)..(25): chemical modification at the 2′ carbon atom with 2′-O-methyl
  misc_feature (27)..(28): chemical modification at the 2′ carbon atom with 2′-O-methyl
  misc_feature (32)..(32): chemical modification at the 2′ carbon atom with 2′-O-methyl
  misc_feature (34)..(35): chemical modification at the 2′ carbon atom with 2′-O-methyl
  misc_feature (37)..(41): chemical modification at the 2′ carbon atom with 2′-O-methyl
  misc_feature (43)..(49): chemical modification at the 2′ carbon atom with 2′-O-methyl
  misc_feature (52)..(52): chemical modification at the 2′ carbon atom with 2′-O-methyl
  misc_feature (54)..(55): chemical modification at the 2′ carbon atom with 2′-O-methyl
  misc_feature (78)..(78): chemical modification at the 2′ carbon atom with 2′-O-methyl
  misc_feature (79)..(79): LNA nucleotide
  misc_feature (80)..(83): chemical modification at the 2′ carbon atom with 2′-O-methyl
  misc_feature (87)..(90): chemical modification at the 2′ carbon atom with 2′-O-methyl
  misc_feature (91)..(94): phosphorothioate modification
  misc_feature (91)..(91): LNA nucleotide
  misc_feature (92)..(93): chemical modification at the 2′ carbon atom with 2′-O-methyl
  misc_feature (94)..(94): LNA nucleotide
  misc_feature (95)..(95): chemical modification at the 2′ carbon atom with 2′-O-methyl
ggugucgagaagaggagaacaauaugcuaaauguuguucucgucuccucgacaccuugucauggaugaccuuggccaggggugccaagcaguugg

SEQ ID NO: 78 - DNA, 95 bp, Artificial Sequence; identical to SEQ ID NO: 77
  GAPDH ORF1 ASO R/G unmod v25
  misc_feature (1)..(3): chemical modification at the 2′ carbon atom with 2′-O-methyl
  misc_feature (1)..(2): phosphorothioate modification
  misc_feature (78)..(78): chemical modification at the 2′ carbon atom with 2′-O-methyl
  misc_feature (79)..(79): LNA nucleotide
  misc_feature (80)..(83): chemical modification at the 2′ carbon atom with 2′-O-methyl
  misc_feature (87)..(90): chemical modification at the 2′ carbon atom with 2′-O-methyl
  misc_feature (91)..(94): phosphorothioate modification
  misc_feature (91)..(91): LNA nucleotide
  misc_feature (92)..(93): chemical modification at the 2′ carbon atom with 2′-O-methyl
  misc_feature (94)..(94): LNA nucleotide
  misc_feature (95)..(95): chemical modification at the 2′ carbon atom with 2′-O-methyl

SEQ ID NO: 79 - DNA, 73 bp, Artificial Sequence
  SERPINA ASO v9.4
  misc_feature (56)..(61): chemical modification at the 2′ carbon atom with 2′-O-methyl
  misc_feature (65)..(73): chemical modification at the 2′ carbon atom with 2′-O-methyl
  misc_feature (69)..(72): phosphorothioate modification
ggugucgagaagaggagaacaauaugcuaaauguuguucucgucuccucgacaccccuuucucgucgaugguc

SEQ ID NO: 80 - DNA, 40 bp, Artificial Sequence
  SERPINA ASO 40nt
  misc_feature (23)..(23): chemical modification at the 2′ carbon atom with 2′-O-methyl
  misc_feature (24)..(24): LNA nucleotide
  misc_feature (25)..(28): chemical modification at the 2′ carbon atom with 2′-O-methyl
  misc_feature (32)..(35): chemical modification at the 2′ carbon atom with 2′-O-methyl
  misc_feature (36)..(39): phosphorothioate modification
  misc_feature (36)..(36): LNA nucleotide
  misc_feature (37)..(38): chemical modification at the 2′ carbon atom with 2′-O-methyl
  misc_feature (39)..(39): LNA nucleotide
  misc_feature (40)..(40): chemical modification at the 2′ carbon atom with 2′-O-methyl
cauggccccagcagcuucagucccuuucucgucgatggtc

SEQ ID NO: 81 - DNA, 95 bp, Artificial Sequence
  SERPINA ASO v25
  misc_feature (1)..(3): chemical modification at the 2′ carbon atom with 2′-O-methyl
  misc_feature (1)..(2): phosphorothioate modification
  misc_feature (5)..(6): chemical modification at the 2′ carbon atom with 2′-O-methyl
  misc_feature (20)..(20): chemical modification at the 2′ carbon atom with 2′-O-methyl
  misc_feature (23)..(23): chemical modification at the 2′ carbon atom with 2′-O-methyl
  misc_feature (25)..(25): chemical modification at the 2′ carbon atom with 2′-O-methyl
  misc_feature (27)..(28): chemical modification at the 2′ carbon atom with 2′-O-methyl
  misc_feature (32)..(32): chemical modification at the 2′ carbon atom with 2′-O-methyl
  misc_feature (34)..(35): chemical modification at the 2′ carbon atom with 2′-O-methyl
  misc_feature (37)..(41): chemical modification at the 2′ carbon atom with 2′-O-methyl
  misc_feature (43)..(49): chemical modification at the 2′ carbon atom with 2′-O-methyl
  misc_feature (78)..(78): chemical modification at the 2′ carbon atom with 2′-O-methyl
  misc_feature (79)..(79): LNA nucleotide
  misc_feature (80)..(83): chemical modification at the 2′ carbon atom with 2′-O-methyl
  misc_feature (87)..(90): chemical modification at the 2′ carbon atom with 2′-O-methyl
  misc_feature (91)..(94): phosphorothioate modification
  misc_feature (91)..(91): LNA nucleotide
  misc_feature (92)..(93): chemical modification at the 2′ carbon atom with 2′-O-methyl
  misc_feature (94)..(94): LNA nucleotide
  misc_feature (95)..(95): chemical modification at the 2′ carbon atom with 2′-O-methyl
ggugucgagaagaggagaacaauaugcuaaauguuguucucgucuccucgacacccauggccccagcagcuucagucccuuucucgucgatggtc

SEQ ID NO: 82 - DNA, 95 bp, Artificial Sequence; identical to SEQ ID NO: 81
  SERPINA ASO v25
  misc_feature (1)..(3): chemical modification at the 2′ carbon atom with 2′-O-methyl
  misc_feature (1)..(2): phosphorothioate modification
  misc_feature (5)..(6): chemical modification at the 2′ carbon atom with 2′-O-methyl
  misc_feature (20)..(20): chemical modification at the 2′ carbon atom with 2′-O-methyl
  misc_feature (23)..(23): chemical modification at the 2′ carbon atom with 2′-O-methyl
  misc_feature (25)..(25): chemical modification at the 2′ carbon atom with 2′-O-methyl
  misc_feature (27)..(28): chemical modification at the 2′ carbon atom with 2′-O-methyl
  misc_feature (32)..(32): chemical modification at the 2′ carbon atom with 2′-O-methyl
  misc_feature (34)..(35): chemical modification at the 2′ carbon atom with 2′-O-methyl
  misc_feature (37)..(41): chemical modification at the 2′ carbon atom with 2′-O-methyl
  misc_feature (43)..(49): chemical modification at the 2′ carbon atom with 2′-O-methyl
  misc_feature (78)..(78): chemical modification at the 2′ carbon atom with 2′-O-methyl
  misc_feature (79)..(79): LNA nucleotide
  misc_feature (80)..(84): chemical modification at the 2′ carbon atom with 2′-O-methyl
  misc_feature (87)..(90): chemical modification at the 2′ carbon atom with 2′-O-methyl
  misc_feature (91)..(94): phosphorothioate modification
  misc_feature (91)..(91): LNA nucleotide
  misc_feature (92)..(93): chemical modification at the 2′ carbon atom with 2′-O-methyl
  misc_feature (94)..(94): LNA nucleotide
  misc_feature (95)..(95): chemical modification at the 2′ carbon atom with 2′-O-methyl

SEQ ID NO: 83 - DNA, 95 bp, Artificial Sequence
  STAT1 ASO v25
  misc_feature (1)..(3): chemical modification at the 2′ carbon atom with 2′-O-methyl
  misc_feature (1)..(2): phosphorothioate modification
  misc_feature (5)..(6): chemical modification at the 2′ carbon atom with 2′-O-methyl
  misc_feature (20)..(20): chemical modification at the 2′ carbon atom with 2′-O-methyl
  misc_feature (23)..(23): chemical modification at the 2′ carbon atom with 2′-O-methyl
  misc_feature (25)..(25): chemical modification at the 2′ carbon atom with 2′-O-methyl
  misc_feature (27)..(28): chemical modification at the 2′ carbon atom with 2′-O-methyl
  misc_feature (32)..(32): chemical modification at the 2′ carbon atom with 2′-O-methyl
  misc_feature (34)..(35): chemical modification at the 2′ carbon atom with 2′-O-methyl
  misc_feature (37)..(41): chemical modification at the 2′ carbon atom with 2′-O-methyl
  misc_feature (43)..(49): chemical modification at the 2′ carbon atom with 2′-O-methyl
  misc_feature (78)..(78): chemical modification at the 2′ carbon atom with 2′-O-methyl
  misc_feature (79)..(79): LNA nucleotide
  misc_feature (80)..(83): chemical modification at the 2′ carbon atom with 2′-O-methyl
  misc_feature (87)..(90): chemical modification at the 2′ carbon atom with 2′-O-methyl
  misc_feature (91)..(91): LNA nucleotide
  misc_feature (91)..(91): phosphorothioate modification
  misc_feature (92)..(93): chemical modification at the 2′ carbon atom with 2′-O-methyl
  misc_feature (92)..(93): phosphorothioate modification
  misc_feature (94)..(94): LNA nucleotide
  misc_feature (94)..(94): phosphorothioate modification
  misc_feature (95)..(95): chemical modification at the 2′ carbon atom with 2′-O-methyl
ggugucgagaagaggagaacaauaugcuaaauguuguucucgucuccucgacacccagacacagaaaucaacucaguctugauacauccaguucc

SEQ ID NO: 84 - DNA, 63 bp, Artificial Sequence; identical to SEQ ID NO: 69
  GAPDH 3′UTR unmod ASO v1

SEQ ID NO: 85 - DNA, 63 bp, Artificial Sequence; identical to SEQ ID NO: 70
  GAPDH 3′UTR unmod ASO v4

SEQ ID NO: 86 - DNA, 73 bp, Artificial Sequence; identical to SEQ ID NO: 71
  GAPDH 3′UTR unmod ASO v9.4

SEQ ID NO: 87 - DNA, 64 bp, Artificial Sequence
  GAPDH sense
ggaccaacugcuuggcaccccuggccaaggucauccaugacaacuuugguaucguggaaggacc

SEQ ID NO: 88 - DNA, 61 bp, Artificial Sequence
  STAT1 sense
gggaacuggaucuaucaagacugaguugauuucugugucugaaguguaagugaacacagaa

SEQ ID NO: 89 - DNA, 61 bp, Artificial Sequence
  SERPINA1 sense
ggaccatcgacgagaaagggactgaagctgctggggccatgtttttagaggccatacccat

SEQ ID NO: 90 - DNA, 21 bp, Artificial Sequence
  ADAR1p150 sense strand
gccucgcgggcgcaaugaatt

SEQ ID NO: 91 - DNA, 21 bp, Artificial Sequence
  ADAR1p150 antisense strand
uucauugcgcccgcgaggcat

SEQ ID NO: 92 - DNA, 22 bp, Artificial Sequence
  5′-UTR SNAP-ADAR
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
ucauuaaacgccagaguccgga

SEQ ID NO: 93 - DNA, 22 bp, Artificial Sequence
  5′-UTR GAPDH isoform 2
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
ucugaauaauccaggaaaagca

SEQ ID NO: 94 - DNA, 22 bp, Artificial Sequence
  ORF #1 GAPDH
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uauaggggugccaagcaguugg

SEQ ID NO: 95 - DNA, 22 bp, Artificial Sequence
  ORF #2 GAPDHb)
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uaugguuuuuccagacggcagg

SEQ ID NO: 96 - DNA, 22 bp, Artificial Sequence
  ORF #1 GUSB
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
ggugcagauuccaggugggacg

SEQ ID NO: 97 - DNA, 22 bp, Artificial Sequence
  ORF #2 GUSB
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
acagacuuggccacugaguggg

SEQ ID NO: 98 - DNA, 22 bp, Artificial Sequence
  3′-UTR SNAP-ADAR
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uaugugucggccacggaacagg

SEQ ID NO: 99 - DNA, 22 bp, Artificial Sequence
  3′-UTR GAPDHc)
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
aauaagggguccacauggcaac

SEQ ID NO: 100 - DNA, 22 bp, Artificial Sequence
  3′-UTR ACTB
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
ucgagcaaugccaucaccuccc

SEQ ID NO: 101 - DNA, 22 bp, Artificial Sequence
  3′-UTR GUSB
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uauuucccugccagaauagaug

SEQ ID NO: 102 - DNA, 22 bp, Artificial Sequence
  KRAS target A/1
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
gaugcuccaaccaccacaaguu

SEQ ID NO: 103 - DNA, 22 bp, Artificial Sequence
  KRAS target 2
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
cgucucuugcccacgccaccag

SEQ ID NO: 104 - DNA, 22 bp, Artificial Sequence
  STAT1 Y701
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
gucucuugauacauccaguucc

SEQ ID NO: 105 - DNA, 22 bp, Artificial Sequence
  5′-GAA
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
cacaugggauucccauugauga

SEQ ID NO: 106 - DNA, 22 bp, Artificial Sequence
  5′-GAU
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uaucgaccaaacccguugacuc

SEQ ID NO: 107 - DNA, 22 bp, Artificial Sequence
  5′-GAC
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
cacgucaugagcccuuccacga

SEQ ID NO: 108 - DNA, 22 bp, Artificial Sequence
  5′-GAG
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
aacgagggaucccgcuccugga

SEQ ID NO: 109 - DNA, 22 bp, Artificial Sequence
  5′-CAA
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
gaagaggcugucgucauacuuc

SEQ ID NO: 110 - DNA, 22 bp, Artificial Sequence
  5′-CAU
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
caagaggucaacgaagggguca

SEQ ID NO: 111 - DNA, 22 bp, Artificial Sequence
  5′-CAC
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
aacgccaggggcgcuaagcagu

SEQ ID NO: 112 - DNA, 22 bp, Artificial Sequence
  5′-CAG
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uacgcauggaccguggucauga

SEQ ID NO: 113 - DNA, 22 bp, Artificial Sequence
  5′-AAA
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uacaugacccucuuggcucccc

SEQ ID NO: 114 - DNA, 22 bp, Artificial Sequence
  5′-AAU
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
gacuagccaaacucguugucau

SEQ ID NO: 115 - DNA, 22 bp, Artificial Sequence
  5′-AAC
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
agucgccacagcuucccggagg

SEQ ID NO: 116 - DNA, 22 bp, Artificial Sequence
  5′-AAG
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uguauauccaccuuaccagagu

SEQ ID NO: 117 - DNA, 22 bp, Artificial Sequence
  5′-UAA
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
aggaggggucucacuccuugga

SEQ ID NO: 118 - DNA, 22 bp, Artificial Sequence
  5′-UAU
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
cuaggcaacaacauccacuuua

SEQ ID NO: 119 - DNA, 22 bp, Artificial Sequence
  5′-UAC
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
ccgagcgccagcagaggcaggg

SEQ ID NO: 120 - DNA, 22 bp, Artificial Sequence
  5′-UAG
  misc_feature (1)..(10): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uaugguuuuuccagacggcagg

SEQ ID NO: 121 - DNA, 22 bp, Artificial Sequence
  5′-CAA methoxy
  misc_feature (1)..(11): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
gaagaggcugucgucauacuuc

SEQ ID NO: 122 - DNA, 22 bp, Artificial Sequence
  5′-CAA fluoro
  misc_feature (1)..(10): chemical modification at the 2′ carbon atom with 2′-O-methyl
  misc_feature (1)..(2): phosphorothioate modification
  misc_feature (11)..(11): chemical modification at the 2′ carbon atom with 2′-fluoro
  misc_feature (14)..(22): chemical modification at the 2′ carbon atom with 2′-O-methyl
  misc_feature (18)..(21): phosphorothioate modification
gaagaggcugucgucauacuuc

SEQ ID NO: 123 - DNA, 22 bp, Artificial Sequence
  5′-AAA methoxy
  misc_feature (1)..(11): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
uacaugacccucuuggcucccc

SEQ ID NO: 124 - DNA, 22 bp, Artificial Sequence
  5′-AAA fluoro
  misc_feature (1)..(10): chemical modification at the 2′ carbon atom with 2′-O-methyl
  misc_feature (1)..(2): phosphorothioate modification
  misc_feature (11)..(11): chemical modification at the 2′ carbon atom with 2′-fluoro
  misc_feature (14)..(22): chemical modification at the 2′ carbon atom with 2′-O-methyl
  misc_feature (18)..(21): phosphorothioate modification
uacaugacccucuuggcucccc

SEQ ID NO: 125 - DNA, 22 bp, Artificial Sequence
  5′-AAC methoxy
  misc_feature (1)..(10): chemical modification at the 2′ carbon atom with 2′-O-methyl
  misc_feature (1)..(2): phosphorothioate modification
  misc_feature (13)..(22): chemical modification at the 2′ carbon atom with 2′-O-methyl
  misc_feature (18)..(21): phosphorothioate modification
agucgccacagcuucccggagg

SEQ ID NO: 126 - DNA, 22 bp, Artificial Sequence
  5′-AAC fluoro
  misc_feature (1)..(10): chemical modification at the 2′ carbon atom with 2′-O-methyl
  misc_feature (1)..(2): phosphorothioate modification
  misc_feature (13)..(13): chemical modification at the 2′ carbon atom with 2′-fluoro
  misc_feature (14)..(22): chemical modification at the 2′ carbon atom with 2′-O-methyl
  misc_feature (18)..(21): phosphorothioate modification
agucgccacagcuucccggagg

SEQ ID NO: 127 - DNA, 22 bp, Artificial Sequence
  5′-UAA methoxy
  misc_feature (1)..(11): chemical modification at the 2′ carbon atom with 2′-O-methyl
  misc_feature (1)..(2): phosphorothioate modification
  misc_feature (14)..(22): chemical modification at the 2′ carbon atom with 2′-O-methyl
  misc_feature (18)..(21): phosphorothioate modification
aggaggggucucacuccuugga

SEQ ID NO: 128 - DNA, 22 bp, Artificial Sequence
  5′-UAA fluoro
  misc_feature (1)..(10): chemical modification at the 2′ carbon atom with 2′-O-methyl
  misc_feature (1)..(2): phosphorothioate modification
  misc_feature (11)..(11): chemical modification at the 2′ carbon atom with 2′-fluoro
  misc_feature (14)..(22): chemical modification at the 2′ carbon atom with 2′-O-methyl
  misc_feature (18)..(21): phosphorothioate modification
aggaggggucucacuccuugga